(19) United States
(12) Patent Application Publication
(10) Pub. No.: US 2016/0220689 A1
(43) Pub. Date: Aug. 4, 2016

(54) MACROMOLECULES OF DENDRIMER-PLATINUM CONJUGATES

(71) Applicant: STARPHARMA PTY LTD., Abbottsford, Victoria (AU)

(72) Inventors: David James Owen, Melbourne (AU); Brian Devlin Kelly, Melbourne (AU); Gareth Rhys Lewis, Melbourne (AU); Rashmi Pathak, Melbourne (AU)

(21) Appl. No.: 14/917,915

(22) PCT Filed: Sep. 10, 2014
(86) PCT No.: PCT/AU2014/000889

§ 371 Date: Mar. 9, 2016

(30) Foreign Application Priority Data
Sep. 10, 2013 (AU) 2013903458

(51) Int. Cl. A61K 47/48; A61K 31/282

(52) U.S. Cl. CPC A61K 47/48215; A61K 31/282; A61K 47/48384; A61K 47/48338

(57) ABSTRACT

A macromolecule comprising a dendrimer having surface amino groups to which at least one platinum-containing moiety is attached is provided. The macromolecule may have a plurality of platinum-containing moieties attached to the surface amino groups. Alternatively, the macromolecule may comprise a dendrimer having surface amino groups to which at least two different moieties are attached, a first moiety being a platinum-containing moiety and the second moiety being a pharmacokinetic modifying agent or targeting agent. Pharmaceutical compositions comprising the macromolecules and uses of the macromolecules for treating or suppressing the growth of a cancer is also described.

### FIELD

The present invention relates to a macromolecule comprising a dendrimer having surface amino groups to which at least one platinum-containing moiety is attached. In particular embodiments, the macromolecule has a plurality of platinum-containing moieties attached to the surface amino groups. In another particular embodiment, the macromolecule comprises a dendrimer having surface amino groups to which at least two different moieties are attached, a first moiety being a platinum-containing moiety and the second moiety being a pharmacokinetic modifying agent or targeting agent. Pharmaceutical compositions comprising the macromolecules and uses of the macromolecules for treating or suppressing the growth of a cancer is also described.

### BACKGROUND

Platinum-containing complexes, such as cisplatin, carboplatin and oxaliplatin, are used clinically in the treatment of cancers including testicular cancer, ovarian cancer, bladder carcinoma, squamous cell carcinoma of the head and neck, non-small cell lung carcinoma, small cell lung carcinoma, cervical carcinoma, colon and colorectal cancer. These clinically approved platinum complexes have platinum in the +2 oxidation state and have a square planar geometry with two cis-oriented inert amine ligands and two cis-oriented labile ligands. The labile ligands are displaced in vivo to generate reactive platinum species that react with DNA and result in cross-linking of DNA. The cross-linking of DNA interrupts cell division processes and causes apoptosis. In addition, research has been conducted on platinum complexes in the +4 oxidation state (Inorg. Chem. 1995, 34, 1015-1021; Chem. Commun., 2012, 48, 847-849 and Coord. Chem. Rev. 2002, 232, 49-67) which convert to the +2 oxidation state on exposure to hypoxic regions associated with solid tumours, as well as the intracellular reducing environment of the cell. Such complexes may contain labile mono dentate and/or bidentate ligands and inert monodentate or bidentate amine ligands in addition to two axial monodentate ligands which influence the reduction kinetics. Three Pt(IV) constructs have entered the clinic, but none have so far shown advantage over their Pt(II) counterparts or gained regulatory approval.

The platinum-containing complexes cause a number of serious side effects that may be dose-limiting or use-limiting. For example, platinum-containing complexes may be neurotoxic and the development of peripheral neurotoxicity is a significant dose-limiting problem which occurs in about 50% of patients receiving cisplatin, 85-95% of patients receiving oxaliplatin and 4-6% of patients receiving carboplatin. Platinum related neurotoxicity presents as both acute, reversible sensitivity to cold and numbness in the hands and feet and a chronic, foot/leg, hand/arm numbness, often with deficits in proprioception and sensory impairment, as well as pharyngolaryngeal dysesthesia.

While carboplatin therapy has a reduced likelihood of causing neurotoxic effects, like all platinum-containing oncology drugs, it has other serious side effects such as myelosuppression, resulting in low blood cell and platelet output from the bone marrow. This side-effect is dose-limiting for carboplatin. Significant decreases in white blood cells (leukopenia and neutropenia) can cause complications such as increased risk of opportunistic infections.

The platinum-containing oncology drugs also cause other side effects including allergic and anaphylactic reactions, hepatotoxicity, pulmonary toxicity, gastrointestinal toxicity, nephrotoxicity and ototoxicity. Tinnitus and hearing loss have been observed in up to 31% of patients treated with cisplatin and there is no effective treatment for this side effect.

Further oxaliplatin used alone is of limited efficacy and is usually used in combination with other oncology drugs such as 5-fluorouracil (5-fu) to achieve effectiveness. Such combination therapies add to the complexity and side effects for patients.

More recently a platinum polymer conjugate, Prolindac™ (AP5346) has commenced Phase II clinical development for ovarian cancer. AP5346 is a linear polymer chain with an average molecular weight of 25 kDa. It is a random copolymer of hydroxypropylmethacrylamide (HPMA) monomer and a methacrylamide linker/chelator conjugate monomer that binds the platinum atom. The copolymer has a ratio of HPMA monomer:linker/chelator monomer of about 10:1. HPMA co-polymer is limited in its loading capacity to about 10% wt. drug. In a Phase I/II study of 26 patients, AP5346 showed no acute onset neurotoxicity, which is a common side effect of oxaliplatin. However, AP5346 uses much greater amounts of platinum to achieve efficacy. The additional platinum increases the cost of the product. In the Phase 1/II study, 46% of patients receiving multiple does of AP5346 reported chronic neurotoxicity, possibly related to the high doses of platinum.

AP5346 has improved targeting of the platinum complex and therefore has reduced side effects compared to non-polymeric platinum complexes. However, AP5346 is a random polymer and its polymeric structure is uncontrolled resulting in a composition comprising a statistical distribution of all possible molecules that can be formed during the random polymerisation reaction. The length of the polymer and the number of linker/chelator monomers and therefore platinum ions that are included can vary from molecule to molecule within the composition. The positions of the linker/chelator monomers may also vary and the folding of the linear polymer may also differ between molecules in the composition which may result in at least some platinum chelators being located internally in the molecule and not being available to bind platinum or the platinum bound not being available for delivery to a tumor.

Furthermore, AP5346 has an average molecular weight of about 25 kDa and therefore is most likely excreted through the kidneys. There is, therefore, a risk of nephrotoxicity with the use of AP5346. It is not possible to increase the size of the AP5346 polymer to minimise nephrotoxicity and improve passive tumour targeting (via enhanced permeability and retention, EPR) without risking toxicity from bioaccumulation of the non biodegradable polymer backbone. HPMA backbone is inherently non-biodegradable and risks cellular bioaccumulation, especially following multiple dose administration regimens.

There is a need for alternative platinum complex-containing molecules with improved side effect profiles, ability to target tumors over normal tissue and that can be readily manufactured with no or reduced polydispersity.

### SUMMARY

The present invention is predicated in part on the discovery that a plurality of platinum moieties can be incorporated into macromolecules comprising dendrimers having surface amino groups. The dendrimeric macromolecules of the invention show favourable side effect profiles, with low neurotoxicity and reduced myelosuppression and/or are effective at controlling or reducing tumors.

In one aspect, there is provided a macromolecule comprising:
i) a dendrimer comprising a core and at least one generation of building units, wherein the outermost generation of building units comprises surface amino groups; and
ii) one or more platinum-containing moiety;
wherein one or more of the surface amino groups are each independently attached to a platinum coordinating group that is coordinated to a platinum atom of a platinum containing moiety.

In another aspect of the invention, there is provided a macromolecule comprising:
i) a dendrimer comprising a core and at least one generation of building units, wherein the outermost generation of building units comprises surface amino groups; and
ii) one or more platinum-containing moiety;
wherein one or more of the surface amino groups are each independently attached to a chelating group that chelates to a platinum atom of a platinum containing moiety.

In an embodiment, the platinum coordinating group comprises a monodentate ligand. In another embodiment, the platinum coordinating group is a chelating group that chelates to a platinum atom of a platinum containing moiety. In another embodiment, the platinum coordinating group is attached to the surface amino group of the dendrimer by a linker group.

In an embodiment, one or more of the surface amino groups are each independently attached to a platinum coordinating group (P_{C}) of a platinum complex -Pc-P_{M} wherein the platinum coordinating group (P_{C}) is coordinated to a platinum atom of the platinum containing moiety (P_{M}).

In an embodiment, one or more of the surface amino groups are each independently attached to a pharmacokinetic modifying agent. The pharmacokinetic modifying agent can be a polyethylene glycol. In an embodiment, the polyethylene glycol has molecular weight in the range of 1000 to 2500 Da.

The macromolecule can further comprise one or more targeting agents attached to one or more surface amino groups of the dendrimer or to a functional group on the core of the dendrimer. In an embodiment, the targeting agent is an antibody or antibody fragment or mimetic.

In an embodiment, the dendrimer of the macromolecule has 2 to 6 generations of building units. In another embodiment, the building units are lysine or lysine analogues. In another embodiment, the macromolecule has an average molecular weight in the range of 60 to 110 kDa.

In an embodiment, platinum-containing moieties are provided on at least about 17.5% of the surface amino groups. In other particular embodiments, there are platinum-containing moieties on at least about 27.5%, 32.5%, 37.5%, 42.5%, 45% or 50% on the surface amino groups.

In another embodiment, the macromolecule is selected from Formula 1:

D_{C}-[D_{B}]_{b}-[D_{S}]ₛ-[L]_{d}-[T]_{z} Formula 1
wherein:
D_{C} represents a core moiety comprising at least two functional groups linked to at least two subsurface building units D_{B};
D_{B} represents two or more subsurface building units each comprising at least three branching point functional groups, wherein one branching point functional group is attached to one of the functional groups of the core or a previous generation subsurface building unit, and at least two functional groups are each independently attached to a next generation of building units; and b is an integer between 2 and 62;
D_{S} represents four or more surface building units wherein each building unit comprises one or more surface amino groups; and s is an integer between 4 and 64;
L is an optional linker group that is attached to the surface amino group of the dendrimer; wherein d is 0 to 128;
T represents one or more terminal groups each independently attached to a surface amino group of the surface building units (Ds), optionally by a linker group L, wherein the one or more terminal groups are selected from at least a first terminal group and optionally a second terminal group, wherein:
   the first terminal group is a platinum complex -Pc-P_{M} wherein a platinum coordinating group (P_{C}) is attached to the surface amino group, optionally by the linker group L, and further coordinated to a platinum atom of the platinum containing moiety (P_{M});
   the second terminal group is selected from at least one of a pharmacokinetic modifying agent (M), a targeting agent, and a blocking agent; and
   z is an integer between 1 and 128.

In an embodiment of Formula 1, the ratio of platinum-containing moieties P_{M} and pharmacokinetic modifying agents M is in the range of 1:4 to 4:1.

In another embodiment of Formula 1, the first terminal group is a platinum complex [-Pc-P_{M}]ₓ comprising a platinum coordinating group (P_{C}) attached to the surface amino group, wherein the platinum coordinating group (P_{C}) is further coordinated to a platinum atom of the platinum containing moiety (P_{M}); and the second terminal group is optionally a pharmacokinetic modifying agent [M]_{y}; and x and z are integers between 1 and 128, and y is an integer between 0 and 127. In a further embodiment, x and d are the same integer of between 1 and 128. In another embodiment, b, s, x and y are together selected from (14, 16, 16, 16), (30, 32, 32, 32) or (62, 64, 64, 64), for a G4, G5 or G6 dendrimer respectively.

In another embodiment, the second terminal group is a pharmacokinetic modifying agent (M) and a targeting agent. It will be appreciated that at least one terminal group T is a platinum complex -Pc-P_{M} wherein a platinum coordinating group (P_{C}) is attached to the surface amino group, optionally by the linker group L, and further coordinated to a platinum atom of the platinum containing moiety (P_{M})

In an embodiment, the platinum coordinating group P_{C} comprises a monodentate or bidentate ligand that can each provide one or more donor atoms for coordination to the platinum atom and are selected from the group consisting of carboxylate, amide, amine, hydroxo and combinations thereof. In another embodiment, the platinum coordinating group is selected from one of:

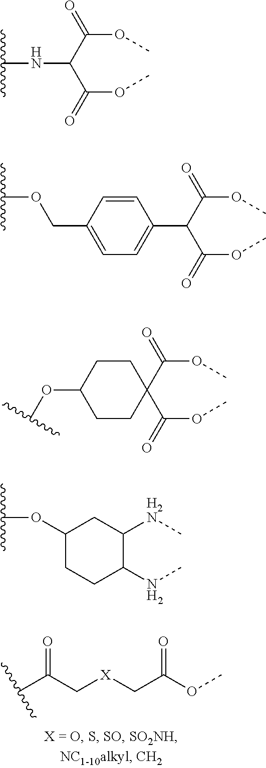

wherein the dashed lines indicate coordination of the monodentate or bidentate ligand to the platinum atom of the platinum containing moiety and the wavy line indicates attachment to a surface amino group of the dendrimer or an optional linking group to the surface amino group of the dendrimer.

In an embodiment, the platinum containing moiety comprises one or two monodentate ligands independently selected from the group consisting of amine and carboxylate groups, or one bidentate ligand selected from the group consisting of diammine, dicarboxylate and amine carboxylate groups. In another embodiment, the platinum-containing moiety P_{M} is selected from the group consisting of:

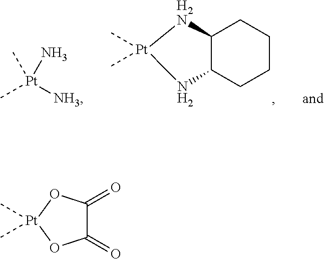

wherein the dashed lines indicate coordination to the platinum coordinating group P_{C}.

In an embodiment, the platinum coordinating group P_{C} and the platinum-containing moiety P_{M} are selected to form a platinum complex -P_{C}-P_{M} that is a four coordinate platinum(II) or a six coordinate platinum(IV) complex. In another embodiment, the four coordinate platinum(II) or six coordinate platinum(IV) complex comprises oxaliplatin, or a moiety or derivative thereof.

In an embodiment, the at least one terminal group of the macromolecule is a six coordinate platinum(IV) complex of:

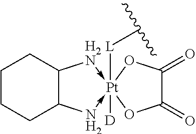

wherein
L is a linker group and the wavy line indicates attachment to a surface amino group of the dendrimer, and
D is a monodentate ligand as defined herein.

In an embodiment, the at least one terminal group of the macromolecule comprises a four coordinate platinum(II) complex of:

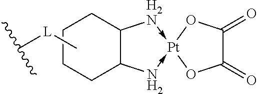

wherein L is a linker group and the wavy line indicates attachment to a surface amino group of the dendrimer.

Each platinum coordinating group can be attached to a surface amino group of the dendrimer through an optional linker group L, for example L-P_{C}-P_{M}. In an embodiment, the linker group L comprises a group of Formula 3:

—C(O)—X—C(O)—W-E Formula 3
wherein one —C(O)— group forms an amide bond with a surface amino group of the dendrimer and the other —C(O)— group, W or E, is attached to the platinum coordinating group P_{C}; and
X is selected from —C₁-C₁₀alkylene-, —(CH₂)_{q}-A-(CH₂)ₜ— and Q;
   A is selected from —O—, —S—, —NR₁—, —N⁺(R₁)₂—, —S—S—, —[OCH₂CH₂]ᵣ—O—, —Y—, and —O—Y—O—;
   Q is selected from Y or —Z═N—NH—S(O)_{w}—Y—;
   Y is selected from cycloalkyl, heterocycloalkyl, aryl and heteroaryl;
   Z is selected from —(CH₂)ₐ—C(CH₃)═, —(CH₂)ₐCH═, cycloalkyl and heterocycloalkyl;
   R₁ is selected from hydrogen and C₁-C₄ alkyl;
   r, q and t are independently selected from 1, 2, 3 and 4;
   w is selected from 0, 1 and 2;
   a is selected from 1, 2, 3 and 4;
   W is absent or is an amino acid residue or a peptide of 1 to 10 amino acid residues; and
   E is absent or a functional group selected from —O—, —S—, —NR₁—, —N⁺(R₁)₂—, —S—S—, —[OCH₂CH₂]ᵣ—O—, —Y—, and —O—Y—O—.

In an embodiment, X can be selected from —C₁₋₆alkylene-, —CH₂-A-CH₂— and —CH₂CH₂-A-CH₂CH₂—. In another embodiment, X is selected from —CH₂CH₂—, —CH₂CH₂CH₂—, —CH₂OCH₂— and —CH₂SCH₂—. In another embodiment, the peptide is selected from -GGG-, -GFLG-, GLFG-, -GILGVP- and -PVGLIG-. In another embodiment, E is —O—.

In another aspect, there is provided a pharmaceutical composition comprising a macromolecule according to any of the above aspects and embodiments and a pharmaceutically acceptable carrier.

In another aspect, there is provided a method of treating or suppressing the growth of a cancer comprising administering an effective amount of a macromolecule or composition thereof according to any of the above aspects and embodiments. In an embodiment, the cancer is a tumor of the prostate, testes, lung, kidney, bladder, colon, pancreas, bone, spleen, liver, brain, head and/or neck, breast, gastrointestinal tract, skin, cervix or ovary.

In another aspect, there is provided a method of reducing the side effects of a platinum-containing oncology drug comprising administering a macromolecule or composition thereof according to any of the above aspects and embodiments. In an embodiment, the side effect that is reduced is hematologic toxicity, neurological toxicity, gastrointestinal toxicity, pulmonary toxicity, cardiotoxicity, hepatotoxicity, nephrotoxicity, ototoxicity or encephalotoxicity.

### DESCRIPTION

### Terms

Singular forms “a”, “an” and “the” include plural aspects unless the context clearly indicates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “optionally substituted” means that a group is either substituted or unsubstituted, at any available position. Substitution can be with one or more groups selected from, e.g. haloalkyl, alkenyl, alkynyl, alkanoic acid, cycloalkyl, cycloalkenyl, formyl, carbonyl, carboxyl, cyano, hydroxy, nitro, alkylthio, alkylthioalkyl, selenoalkyl, sulfonyl, alkylsulfonyl, alkoxy, amino, aminoalkyl and amidoalkyl. It will be appreciated that other groups not specifically described may also be used. In one embodiment, “optionally substituted” means substitution with one or more groups selected from halo, OH, C═O, COOH, C₁₋₄alkyl, C₁₋₄alkanoic acid, thiolC₁₋₄alkyl, C₁₋₄heterocyclyl, selenylC₁₋₄alkyl, aminoC₁₋₄alkyl, amidoC₁₋₄alkyl, hydroxyC₁₋₄alkyl.

The term “halo” or “halogen” whether employed alone or in compound words such as haloalkyl, haloalkoxy or haloalkylsulfonyl, represents fluorine, chlorine, bromine or iodine. Further, when used in compound words such as haloalkyl, haloalkoxy or haloalkylsulfonyl, the alkyl may be partially halogenated or fully substituted with halogen atoms which may be independently the same or different. Examples of haloalkyl include, without limitation, —CH₂CH₂F, —CF₂CF₃ and —CH₂CHFCl. Examples of haloalkoxy include, without limitation, —OCHF₂, —OCF₃, —OCH₂CCl₃, —OCH₂CF₃ and —OCH₂CH₂CF₃. Examples of haloalkylsulfonyl include, without limitation, —SO₂CF₃, —SO₂CCl₃, —SO₂CH₂CF₃ and —SO₂CF₂CF₃.

“Hydroxy” represents a —OH moiety. “Carboxyl” represents a —CO₂H moiety. “Cyano” represents a —CN moiety. “Carbonyl” represents a —(C═O)— moiety. “Amino” represents an —NH₂ moiety.

Alkoxy” represents an —O-alkyl group in which the alkyl group is as defined supra. Examples include methoxy, ethoxy, n-propoxy, iso-propoxy, and the different butoxy, pentoxy, hexyloxy and higher isomers.

“Aminoalkyl” represents an —R—NH₂, —R—NHR or —NR₂ group in which R is an alkyl group as defined supra. Examples include, without limitation, methylamine, ethylamine, n-propylamine, ethyldimethylamine, and different and higher isomers.

“Sulfonyl” represents an —SO₂R group that is linked to the rest of the molecule through a sulfur atom.

As used herein, the term “alkyl” refers to a straight chain or branched saturated hydrocarbon group having 1 to 10 carbon atoms. Where appropriate, the alkyl group may have a specified number of carbon atoms, for example, C₁₋₄alkyl which includes alkyl groups having 1, 2, 3 or 4 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl and decyl.

The term “alkylene” as used herein refers to a straight-chain divalent alkyl group having 1 to 10 carbon atoms. Where appropriate, the alkylene group may have a specified number of carbon atoms, for example C₁-C₆ alkylene includes —CH₂—, —(CH₂)₂—, —(CH₂)₃—, —(CH₂)₄—, —(CH₂)₅ and —(CH₂)₆—.

As used herein, the term “cycloalkyl” refers to a saturated or unsaturated cyclic hydrocarbon. The cycloalkyl ring may include a specified number of carbon atoms. For example, a 3 to 8 membered cycloalkyl group includes 3, 4, 5, 6, 7 or 8 carbon atoms. Examples of suitable cycloalkyl groups include, but are not limited to, cyclopropyl, cyclobutyl, cyclopentanyl, cyclopentenyl, cyclohexanyl, cyclohexenyl, 1,4-cyclohexadienyl, cycloheptanyl and cyclooctanyl.

As used herein, the term “aryl” is intended to mean any stable, monocyclic or bicyclic carbon ring of up to 7 atoms in each ring, wherein at least one ring is aromatic. Examples of such aryl groups include, but are not limited to, phenyl, naphthyl, tetrahydronaphthyl, indanyl, biphenyl and binaphthyl.

The term “heterocycloalkyl” or “heterocyclyl” as used herein, refers to a cyclic hydrocarbon in which one to four carbon atoms have been replaced by heteroatoms independently selected from the group consisting of N, N(R), S, S(O), S(O)₂ and O. A heterocyclic ring may be saturated or unsaturated. Examples of suitable heterocyclyl groups include tetrahydrofuranyl, tetrahydrothiophenyl, pyrrolidinyl, pyrrolinyl, pyranyl, piperidinyl, pyrazolinyl, dithiolyl, oxathiolyl, dioxanyl, dioxinyl, morpholino and oxazinyl.

The term “heteroaryl” as used herein, represents a stable monocyclic or bicyclic ring of up to 7 atoms in each ring, wherein at least one ring is aromatic and at least one ring contains from 1 to 4 heteroatoms selected from the group consisting of O, N and S. Heteroaryl groups within the scope of this definition include, but are not limited to, acridinyl, carbazolyl, cinnolinyl, quinoxalinyl, quinazolinyl, pyrazolyl, indolyl, benzotriazolyl, furanyl, thienyl, thiophenyl, 3,4-propylenedioxythiophenyl, benzothienyl, benzofuranyl, benzodioxane, benzodioxin, quinolinyl, isoquinolinyl, oxazolyl, isoxazolyl, imidazolyl, pyrazinyl, pyridazinyl, pyridinyl, pyrimidinyl, pyrrolyl, tetrahydroquinoline, thiazolyl, isothiazolyl, 1,2,4-triazolyl, 1,2,3-triazolyl, 1,2,4-oxadiazolyl, 1,2,4-thiadiazolyl, 1,3,5-triazinyl, 1,2,4-triazinyl, 1,2,4,5-tetrazinyl and tetrazolyl, dihydropyrazinyl and dihydropyrazolyl.

The term “dendrimer” refers to a molecule containing a core and at least one dendron attached to the core. Each dendron is made up of at least one layer or generation of branched building units resulting in a branched structure with increasing number of branches with each generation of building units. The maximum number of dendrons attached to the core is limited by number of functional groups on the core. The core may have one or more functional groups suitable to bear a dendron and optionally an additional functional group for attachment of an agent suitable for modifying pharmacokinetics or targeting organs or tissues, as well as a second drug moiety.

The term “building unit” used herein refers to a branched molecule having at least three functional groups, one for attachment to the core or a previous generation of building units and at least two functional groups for attachment to the next generation of building units or forming the surface amino groups of the dendrimer. It will be appreciated that the term “building unit” may be generally used to refer to a “subsurface building unit” (D_{B}) or a “surface building unit” (D_{S}) as described herein.

The term “generation” refers to the number of layers of building units that make up a dendron or dendrimer. For example, a one generation dendrimer (G1) will have one layer of branched building units attached to the core, for example, Core-[[building unit]]ᵤ where u is the number of dendrons attached to a core having u or greater than u number of functional groups. For example, u can be 1 to 4, or 2 or 3. A two generation dendrimer (G2) has two layers of building units in each dendron attached to the core, for example, when the building unit has one branch point, the dendrimer may be: Core[[building unit][building unit]₂]ᵤ, a three generation dendrimer (G3) has three layers of building units in each dendron attached to the core, for example Core-[[building unit][building unit]₂[building unit]₄]ᵤ, a six generation dendrimer (G6) has six layers of building units attached to the core, for example, Core-[[building unit][building unit]₂[building unit]₄[building unit]₈[building unit]₁₆[building unit]₃₂]ᵤ, and the like. The last generation of building units (the outermost generation) also referred to herein as “surface building units”, provides the surface amino functionalisation of the dendrimer (and the number of amino functional groups available for binding terminal groups), which are referred to herein as “surface amino groups”. For example, in a dendrimer having a core with two dendrons attached (u=2), if each building unit has one branch point and there are 6 generations, the outermost generation has 64 building units and 128 amino functional groups forming the surface amino groups of the dendrimer and are available to bind terminal groups (and the dendrimer has a further 62 subsurface building units).

The term “terminal group” is a collective term to refer to one or more of a linker, platinum chelating group, platinum co-ordinating group, platinum-containing moiety, pharmacokinetic modifying agent, targeting agent or blocking group that can be attached to the surface amino groups of the dendrimer.

The term “surface amino group” refers to a functional group on the outermost generation of building units as described herein, which comprises a reactive nitrogen species capable of attachment to a terminal group such as a linking, platinum chelating or platinum co-ordinating group (for further attachment of a platinum-containing moiety), or attachment of other terminal group as described herein. For example, a “surface amino group” can be provided by a primary or secondary amine group, which can be attached to a linking, co-ordinating or chelating group (e.g. by reaction with a carboxylic acid of a linking group to form an intervening amide functionality or by reacting an azide with an alkyne to form a triazole).

As used herein, the term “lysine analogue” refers to a moiety having a carboxy group that reacts with an amino group of the core or an earlier generation of building units and at least two amino groups that react with the carboxy group of a subsequent generation of building units or form the surface amino groups of the dendrimer.

The term “platinum coordinating group” refers to a group comprising a ligand that is capable of binding to a platinum atom. The platinum coordinating group can also comprise a functional group for attachment to the surface amino group (e.g. carboxylic acid group) or for attachment to the optional linker group. It will be appreciated that such ligands include monodentate ligands, or chelating ligands from a chelating group such as a bidentate ligand.

It will be appreciated that the term “monodentate ligand” or variant thereof refers to a ligand which binds to a platinum atom at one site of the platinum atom. The term “bidentate ligand” or variant thereof refers to a ligand which binds to a platinum atom at two sites of the platinum atom. It is understood that as used herein, a monodentate or bidentate ligand may contain additional atoms capable of binding to a platinum atom, but wherein the ligand binds the platinum atom with only the indicated number of sites. For example, a ligand containing at least two nitrogens would be considered a monodentate ligand if only one nitrogen binds to the platinum atom, and would be considered a bidentate ligand if both nitrogens independently bind to the platinum atom. The term “polydentate ligand” or variant thereof refers to a ligand which binds to a platinum atom at two or more sites of the platinum atom.

The term “chelating group” refers to a group comprising a polydentate ligand capable of coordination at two or more sites of a platinum atom. For example, the chelating group may comprise a bidentate or tridentate ligand. In one embodiment, the chelating group comprises a bidentate ligand.

The term “labile” indicates that the bond between the ligand, coordinating group, or chelating group and the platinum atom can be replaced by another group, such as water, in vivo. The term “inert” indicates that the bond between the ligand, linker coordinating group, or chelating group and the platinum atom is not readily replaced by another group, in vivo.

As used herein “platinum-containing moiety” refers to a group comprising a platinum atom. For example, the platinum containing moiety can be a platinum complex comprising a platinum atom coordinated to one or more ligands, and wherein the platinum atom can be further coordinated to at least one ligand of the platinum coordinating group or chelating group as described herein, such as to form four or six coordinate platinum complexes that are attached, optionally by a linker, to the dendrimers of the macromolecules.

As used herein “platinum-containing oncology drug” refers to compound that is suitable for use as an oncology drug and comprises a complexed platinum ion. The platinum-containing oncology drug may be monomeric, containing a single complexed platinum atom or may be polymeric, containing multiple platinum complexes incorporated into a polymer. Examples of monomeric platinum-containing oncology drugs include cisplatin, carboplatin, oxaliplatin, nedaplatin, lobaplatin and heptaplatin. Examples of polymeric platinum-containing oncology drugs include AP5346. The term platinum-containing oncology drug as used herein does not include the platinum-containing dendrimeric macromolecules of the present invention.

The term “polyethylene glycol” or “PEG” as used herein refers to a polyalkylene glycol compound or derivative thereof, with or without coupling agents or derivatization with coupling or activating moieties. In its typical form, PEG is a linear polymer with terminal hydroxyl groups and has the formula HO—CH₂CH₂—(CH₂CH₂O)n-CH₂CH₂—OH. The number of repeating subunits “n” in the PEG is approximated for the molecular mass described in Daltons. Typically, PEG reagents used to prepare PEGylated compounds comprise a heterogenous mixture of PEGs having a different number (n) of ethylene glycol subunits in the PEG polymer. A single ethylene glycol subunit (—(CH₂CH₂O)) of PEG has a molecular weight of about 44 Daltons. Therefore, the molecular weight of the PEG polymer depends on the number (n). Peg may be linear or branched. Branched Peg include trident PEG.

Numerous derivatives of PEG and methods for making them and conjugating them to a protein such as insulin are known in the art and are suitable for use in the present invention. See, e.g., PCT International Patent Application Pub. Nos. WO 01/62827, WO 2006/028745, WO 2006/096535, WO 2006/036825; Zalipsky, S. Bioconjugate Chem. 6:150-165, 1995; Veronese, et al., Applied Biochem. and Biotech. 11:141-152, 1985; and Roberts, M. et al. Advanced Drug Delivery Reviews, 54:459-476, 2002. PEGs as used are typically mixtures of PEG compounds, the molecular weight of a PEG describes the average size of the PEG reagent used.

Three methods are commonly used to calculate MW averages: number average, weight average, and z-average molecular weights. As used herein, the phrase “molecular weight” is intended to refer to the weight-average molecular weight which can be measured using techniques well-known in the art including, but not limited to, NMR, mass spectrometry, matrix-assisted laser desorption ionization time of flight (MALDI-TOF), gel permeation chromatography or other liquid chromatography techniques, light scattering techniques, ultracentrifugation and viscometry.

The ratio of weight average molecular weight and number average molecular weight is known as the polydispersity index (PDI), and provides a rough indication of the breadth of the distribution. PEG reagents are typically polydisperse (i.e., number average molecular weight and weight average molecular weight of the polymers are not equal). The PDI for PEG reagents used to prepare the compounds or compositions of the present invention may be less than about 1.1 or less than about 1.05.

It will be clearly understood that, although a number of prior art publications are referred to herein, these references do not constitute an admission that any of these documents form part of the common general knowledge in the art, in Australia or in any other country.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The macromolecules of the invention may be in the form of pharmaceutically acceptable salts. It will be appreciated however that non-pharmaceutically acceptable salts also fall within the scope of the invention since these may be useful as intermediates in the preparation of pharmaceutically acceptable salts or may be useful during storage or transport. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benzenesulphonic, salicyclic sulphanilic, aspartic, glutamic, edetic, stearic, palmitic, oleic, lauric, pantothenic, tannic, ascorbic and valeric acids. Base salts include, but are not limited to, those formed with pharmaceutically acceptable cations, such as sodium, potassium, lithium, calcium, magnesium, ammonium and alkylammonium. Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

### Macromolecules

In one aspect, there is provided a macromolecule comprising:
i) a dendrimer comprising a core and at least one generation of building units, wherein the outermost generation of building units comprises surface amino groups; and
ii) one or more platinum-containing moiety;
wherein one or more of the surface amino groups are each independently attached to a platinum coordinating group that is coordinated to a platinum atom of the platinum containing moiety.

The platinum coordinating group may be a chelating group. In an embodiment there is provided a macromolecule comprising:
i) a dendrimer comprising a core and at least one generation of building units, wherein the outermost generation of building units comprises surface amino groups; and
ii) one or more platinum-containing moieties;
wherein one or more of the surface amino groups are each independently attached to a chelating group that chelates to a platinum atom of a platinum containing moiety.

It will be appreciated that the platinum coordinating group provides an attachment to a surface amino group and provides at least one ligand that is attached to a platinum atom of a platinum containing moiety. For example, a platinum complex [Pc-P_{M}] is formed from the attachment of at least one ligand of the platinum coordinating group (P_{C}) with the platinum containing moiety (P_{M}). It will also be appreciated that further ligands for the platinum complex are provided by the platinum containing moiety.

In one embodiment, one or more of the surface amino groups are each independently attached to a platinum coordinating group (P_{C}) of a platinum complex -Pc-P_{M} wherein the platinum coordinating group (P_{C}) is coordinated to a platinum atom of the platinum containing moiety (P_{M}). In another embodiment, the platinum complex is a four or six coordinate platinum complex.

The platinum coordinating group comprises a monodentate ligand or chelating group, for example a bidentate ligand. In one embodiment, the platinum coordinating group is a chelating group. In another embodiment, the platinum coordinating group is a monodentate ligand or a bidentate ligand. In yet a further embodiment, the platinum coordinating group is a bidentate ligand. The platinum coordinating group may be attached to the dendrimer by a linking group as described below. For example, the linking group may comprise the platinum coordinating group.

It will be appreciated that the platinum coordinating group, which comprises a monodentate ligand or chelating group, may contain additional atoms capable of binding to the platinum atom, but wherein the ligand binds to the platinum atom at only one site for the monodentate ligand and at two sites the “bidentate ligand”. For example, a platinum coordinating group containing two or more coordinating nitrogen atoms would be considered a monodentate ligand if only one nitrogen atom of the ligand binds to the platinum atom, and would be considered a bidentate ligand if two nitrogen atoms of the ligand bind to the platinum atom.

In another embodiment, the macromolecule is selected from Formula 1:

D_{C}-[D_{B}]_{b}-[D_{S}]ₛ-[L]_{d}-[T]_{z} Formula 1
wherein:
D_{C} represents a core moiety comprising at least two functional groups linked to at least two subsurface building units D_{B};
D_{B} represents two or more subsurface building units each comprising at least three branching point functional groups, wherein one branching point functional group is attached to one of the functional groups of the core or a previous generation subsurface building unit, and at least two functional groups are each independently attached to a next generation of building units; and b is an integer between 2 and 62;
D_{S} represents four or more surface building units wherein each building unit comprises one or more surface amino groups; and s is an integer between 4 and 64;
L is an optional linker group that is attached to the surface amino group of the dendrimer; wherein d is 0 to 128;
T represents one or more terminal groups each independently attached to a surface amino group of the surface building units (Ds), optionally by a linker group L, wherein the one or more terminal groups are selected from at least a first terminal group and optionally a second terminal group, wherein:
   the first terminal group is a platinum complex -Pc-P_{M} wherein a platinum coordinating group (P_{C}) is attached to the surface amino group, optionally by the linker group L, and further coordinated to a platinum atom of the platinum containing moiety (P_{M});
   the second terminal group is selected from at least one of a pharmacokinetic modifying agent (M), a targeting agent, and a blocking agent; and
   z is an integer between 1 and 128.

In another embodiment of Formula 1, the first terminal group is a platinum complex [-Pc-P_{M}], comprising a platinum coordinating group (P_{C}) attached to the surface amino group, wherein the platinum coordinating group (P_{C}) is further coordinated to a platinum atom of the platinum containing moiety (P_{M}); and the second terminal group is optionally a pharmacokinetic modifying agent [M]_{y}; and x and z are integers between 1 and 128, and y is an integer between 0 and 127. In a further embodiment, x and d are both the same integer of between 1 and 128. In another embodiment, b, s, x and y are together selected from (14, 16, 16, 16), (30, 32, 32, 32) or (62, 64, 64, 64), for a G4, G5 or G6 dendrimer respectively. It will be appreciated that Formula 1 is a simplified representation of a complex 3 dimensional multi branched structure. The subsurface building units are those building units which do not provide surface functional groups.

The integers for d and x may be selected to provide a macromolecule having at least 17.5% of the surface amino groups bound to platinum containing moiety P_{M} via a linker group L and platinum coordinating group P_{C} (i.e. at least 50% terminal groups being a group L-P_{C}-P_{M}). In an embodiment, d and x are selected to provide at least 50% of the terminal groups being a group L-P_{C}-P_{M}. In a particular embodiment, d and x are selected to provide at least 75% of the terminal groups being a group L-P_{C}-P_{M}. Where the dendrimer is a generation 5 (G5) polylysine dendrimer, x and d may be at least 10, at least 15, at least 20, or at least 25. In some embodiments, the pharmacokinetic modifying agent M is bound to at least about 15%, 25%, 30%, 35%, 40%, 45% or 50% of the total number of surface amino groups. Where dendrimer is a G5 polylysine dendrimer, y can be at least 10, at least 15, at least 20, or at least 25.

The subsurface layers of building units can be described with more detail using the nomenclature above in the discussion of the term “generation”. For example for a generation three dendrimer:

Core-[[building unit]ₙ[building unit]ₘ[surface building unit]ₚ]ᵤ
wherein n≦m≦p; n is 1; m is an integer between 1 and 4; p is an integer between m and 16; and u is an integer between 1 and 4. For example, where u=3 (i.e. the core is tri-functional and has 3 dendrons attached) and each building unit has one branch point then n=1, m=2, p=4, and the total number of building units is 21, consisting of 12 surface and 9 subsurface building units. In will be appreciated that this example can be extended to dendrimers with different number of generations and dendrons. For example generation five (G5) dendrimer having 2 dendrons and using lysine (lys) for the 62 building units could be represented as:

Core [[Lys][Lys]₂[Lys]₄[Lys]₈[Lys]₁₆]₂

or alternatively:

Core[Lys]₂[Lys]₄[Lys]₈[Lys]₁₆[Lys]₃₂

### Platinum Containing Moiety (P_{M})

According to the above aspects, the dendrimer has at least one platinum-containing moiety (P_{M}) attached to its surface through a platinum coordinating group (P_{C}). In other embodiments, the dendrimer has all of its surface amino groups each attached to a platinum-containing moiety through a platinum coordinating group. In yet other embodiments, there is more than one platinum-containing moiety attached to the surface of the dendrimer through a platinum coordinating group but less than the maximum number that could be attached to the surface amino groups of the dendrimer. In particular embodiments, there are platinum-containing moieties on more than 24% and/or less than 76% of the surface amino groups. In particular embodiments there are platinum-containing moieties on at least about 17.5%, 32.5%, 37.5%, 42.5%, 45% or 50% on the surface amino groups.

When more than one platinum-containing moiety is attached to the surface of the dendrimer, the platinum-containing moieties may be the same or different. In particular embodiments, the platinum-containing moieties are the same.

It will be appreciated that the platinum coordinating group is attached to a surface amino group of the dendrimer, optionally by a linker group, and further provides at least one ligand that is attached to a platinum atom of the platinum containing moiety. In other words, a platinum complex is formed from the attachment of at least one ligand of the platinum coordinating group with the platinum containing moiety. It will also be appreciated that further ligands for the platinum complex are provided by the platinum containing moiety. In one embodiment, the platinum complex is a four or six coordinate platinum complex.

It will also be appreciated that ligands coordinate to the platinum atom by interactions of the platinum atom's d orbital with the s or p orbitals of the ligand(s). For example, typical oxidation states for platinum are +2 and +4, and platinum is typically coordinated to 4 or 6 ligands depending on the type of ligands. The geometries of platinum complexes may vary, for example platinum is typically square planar when coordinated to 4 ligands and octahedral when coordinated to 6 ligands. In one embodiment, the platinum-containing moiety, when attached to the surface amino groups by the platinum coordinating group, comprises a four coordinate platinum atom in a +2 oxidation state. In another embodiment, the platinum-containing moiety, when attached to the surface amino groups by the platinum coordinating group, comprises a six coordinate platinum atom in a +4 oxidation state.

The platinum containing moiety (P_{M}), or platinum complex [Pc-P_{M}] comprising a platinum coordinating group (P_{C}) coordinated to a platinum-containing moiety (P_{M}), may comprise a platinum compound, or moiety thereof, of a monomeric platinum-containing oncology drug, for example cisplatin, carboplatin, oxaliplatin, nedaplatin, lobaplatin and heptaplatin. The platinum containing moiety (P_{M}) or platinum complex [Pc-P_{M}] may comprise one or more ligands or one or more chelating groups of a monomeric platinum-containing oncology drug. It will be appreciated a monomeric platinum-containing oncology drug, or moiety thereof, may be attached to a linker group or platinum coordinating group at a number of sites, for example by coordinating to the platinum atom of P_{M} or by attachment to a ligand on P_{M} or P_{C}.

It will be appreciated that oxaliplatin is a compound of the following structure:

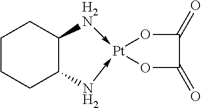

In an embodiment, the platinum containing moiety is oxaliplatin, or a derivative or moiety thereof. For example, the platinum containing moiety P_{M}, platinum coordinating group P_{C}, or platinum complex P_{C}-P_{M}, may each independently comprise at least one ligand selected from diaminocyclohexane and oxalate, each of which may be optionally substituted with one or more substituents selected from halo, OH, C₁₋₆alkyl, OC₁₋₆alkyl or OC(O)C₁₋₆alkyl. The platinum containing moiety P_{M}, platinum coordinating group P_{C}, or platinum complex P_{C}-P_{M}, may comprise one or more ligands independently selected from the group consisting of an amine, carboxylate, hydroxyl and halo, which coordinate to the platinum atom. In an embodiment, platinum containing moiety P_{M}, platinum coordinating group P_{C}, or platinum complex P_{C}-P_{M}, may comprise one or more ligands independently selected from the group consisting of an amine and carboxylate, which coordinate to the platinum atom.

In an embodiment, the platinum containing moiety P_{M}, platinum coordinating group P_{C}, or platinum complex P_{C}-P_{M}, may comprise a derivative of oxaliplatin provided by a compound of the following structure:

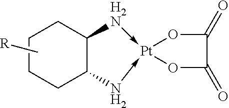

wherein R can be independently selected from the group consisting of hydrogen, halo, OH, C₁₋₆alkyl, OC₁₋₆alkyl, COOH, or OC(O)C₁₋₆alkyl. R may also provide a functional group for attachment to a linking group, or when the above structure is a platinum containing moiety P_{M} then a platinum coordinating group can coordinate to the platinum atom.

In particular embodiments, the platinum-containing moiety P_{M} is selected from one of:

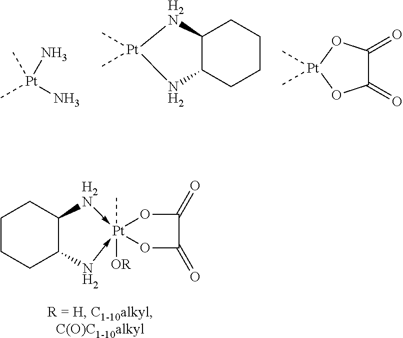

wherein the dashed lines indicate coordination with the monodentate or bidentate ligands of the platinum coordinating group according to the aspects described above.

In some embodiments the one or more ligands of the platinum coordinating group (P_{C}) that are attached to the platinum containing moiety (P_{M}) in forming the platinum complex (P_{C}-P_{M}) are selected from labile ligands such that the platinum containing moiety (P_{M}) is released from the macromolecule for activity in suitable environments in vivo, such as reducing, acidic or hypoxic environments. In other embodiments the platinum containing moiety is released from the dendrimer by cleavable groups on the platinum coordinating group or optional linker group.

The platinum complexes described herein may include salts, solvates, hydrates, isomers, tautomers, racemates, stereoisomers, enantiomers or diastereoisomers of those complexes. For example, it will be appreciated that the platinum complexes include isomers that have different connectivity between ligand groups, such as where a four ligand coordinated metal complex can exist in a cis or trans form, and reference herein to a cis form is also intended to cover the trans form and vice versa.

### Linking Group, Chelating Group and Ligands

It will be appreciated that in the macromolecules as described herein, a ligand for the platinum atom (which atom is contained in the platinum containing moiety P_{M}) is provided by at least one ligand of the platinum coordinating group P_{C}. It will also be appreciated that one or more ligands for the platinum atom are provided by the platinum containing moiety itself. Any of the ligands for the platinum atom can be independently selected from one or more monodentate ligands, one or more polydentate or chelating (including bidentate) ligands, or a combination thereof. Each ligand may be the same or different. It will be appreciated that a monodentate ligand group provides a single ligand for coordination to the platinum atom, and a bidentate ligand group provides two ligands for coordination to the platinum atom. For example, when platinum is four coordinate (coordinated to 4 donor ligands in total), this can be provided by two bidentate ligands each providing two donor atoms for coordination to the platinum (e.g. a bidentate ligand from the platinum containing moiety and a bidentate ligand from the platinum coordinating group), by four monodentate ligands each providing a single donor atom (e.g. one monodentate ligand of the platinum coordinating group and three monodentate ligands from the platinum containing moiety), or by a combination of one bidentate ligand and two monodentate ligands (e.g. one bidentate ligand of the platinum coordinating group and two monodentate ligands of the platinum containing moiety).

The monodentate or bidentate ligands can comprise one or more donor atoms for coordination to the platinum atom selected from the group consisting of nitrogen, oxygen, sulphur, and combinations thereof. The monodentate or bidentate ligands can comprise one or more donor atoms for coordination to the platinum atom provided in the groups selected from amine, amide, carboxyl, carboxylate, cyano, hydroxo or combinations thereof. In a particular embodiment, the monodentate or bidentate ligands comprise one or more donor atoms for coordination to the platinum atom selected from the group consisting of carboxylate, amide, amine, hydroxo and combinations thereof.

Suitable monodentate or polydentate ligands for the platinum containing moiety or the platinum coordinating group are provided in the below.

Monodenate ligands

| | | | |
|---|---|---|---|
| F⁻ | Fluoro | OH⁻ | Hydroxo |
| Cl⁻ | Chloro | SO₄²⁻ | Sulfato |
| Br⁻ | Bromo | S₂O₃²⁻ | Thiosulfato |
| I⁻ | Iodo | NO₂⁻ | Nitrito-N-; Nitro |
| O²⁻ | Oxo | ONO⁻ | Nitrito-O-; Nitrito |
| CN⁻ | Cyano | SCN⁻ | Thiocyanato-S-; Thiocyanato |
| NC− | Isocyano | NCS⁻ | Thiocyanato-N-; Isothiocyanato |
| NH₃ | Ammine | H₂O | Aqua |
| CO | Carbonyl | NO | Nitrosyl |
| CH₃NH₂ | Methylamine | C₅H₅N | Pyridine |

Polydentate ligands

| | |
|---|---|
| —OC(O)C(O)O— | Oxalate ion (ox) |
| —OC(O)CH₂C(O)O— | Malonate ion |
| CH₃C(O)CH═C(O⁻)CH₃ | Acetylaetonate (acac) |
| (C₆H₅N)₂ | 2,2′Dipyridyl (dipy) |
| NH₂CH₂CH₂NH₂ | Ethylene diamine |
| o-Phen | ortho-Phenanthroline |

The platinum coordinating group (P_{C}) and the platinum containing moiety (P_{M}) may comprise one or more monodentate or polydentate ligands as described above or a derivative thereof. For example, the platinum complex (P_{C}-P_{M}), which is formed from the platinum coordinating group (P_{C}) and the platinum containing moiety (P_{M}), may comprise one or more of the above described monodentate or polydentate ligands. In one embodiment, the platinum complex comprises at least two bidentate ligands to form a four or six coordinate platinum complex.

In one embodiment, the ligands are independently selected front one or more monodentate ligands of halo, SR², OC(═O)R², SC(═O)R², OC(═S)R², SC(═S)R², or a bidentate ligand formed two of the groups joined to form an optionally substituted 4-8 membered ring optionally containing 1-4 heteroatoms selected from N, O and S which is optionally fused with a 4, 5 or 6 membered ring. R² is C₁₋₁₀alkyl optionally substituted with 1-3 substituents independently selected from halo, OH and COOH;

Monodentate ligands may be selected from halo and optionally substituted heteroalkyls such as optionally substituted alkylthiols, alkanoates, thiolalkanoates and alkoxyalkanoates, such as a carboxylated PEG ether. A monodentate ligand may be halo, such as F, Cl, Br or I. In one embodiment, the monodentate ligand is Cl.

A monodentate ligand may be an optimally substituted C₂₋₈alkanoate, which may be generally represented by an OC(═O)—R group in which R is an optionally substituted alkyl group as defined herein consisting of the stated number of carbon atoms, and in which may provide a monodentate ligand as defined herein joined to the platinum atom through its terminal oxygen atom. For example, the monodentate ligand may be an optionally substituted ethanoate, propanoate, butanoate, pentanoate, hexanoate, septanoate, or octanoate. The monodentate ligand may be an optionally substituted hydroxyC₂₋₈alkanoate, such as an α or β hydroxyC₂₋₈alkanoate, for example 2-hydroxypropanoate.

A bidentate ligand can be provided by a two monodentate ligands as described above attached to form an optionally substituted 4-8 membered ring optionally containing 1-4 heteroatoms selected from N, O and S, and wherein the 4-8 membered ring is optionally fused with a 4, 5 or 6 membered ring. The bidentate ligand L-L may be an optionally substituted Y—(CR¹²R¹³)ₘ—Y group, which provides a bidentate ligand as defined herein joined to the platinum atom M through each of its two terminal atoms of the Y group, and wherein each Y is independently selected from OC(O), NR¹¹ and O, m is an integer of 1 to 6, R¹¹, R¹² and R¹³ are each independently selected from hydrogen, OH, COOH, optionally substituted C₁₋₆alkyl, optionally substituted C₃₋₆cycloalkyl and an optionally substituted monocyclic aryl or heteroaryl ring, or R¹¹ may be joined with one of R¹² and R¹³ to form a 5 or 6 membered ring. The optional substitution of the C₁₋₆alkyl group may be selected from halo, OH, COOH, SH, NH(C═NH)NH₂, C(O)NH and optionally substituted monocyclic or bicyclic aryl or hetaryl.

In one embodiment, the bidentate ligand is OC(═O)—C(═O)O. The OC(═O)—C(═O)O group may be referred to as an oxalate group, which provides a bidentate ligand as defined herein joined to the metal atom M through each of its two terminal oxygen atoms.

In another embodiment the bidentate ligand L-L is OC(═O)(CR¹²R¹³)ₘ—C(═O)O, which provides a bidentate ligand as defined herein joined to the platinum atom through each of its two terminal oxygen atoms, and wherein m is an integer of 1 to 6, each R¹² and R¹³ are independently selected from hydrogen, OH, COOH, optionally substituted C₁₋₆alkyl, optionally substituted C₃₋₆cycloalkyl and an optionally substituted monocyclic aryl or heteroaryl ring. In one embodiment, R¹² is hydrogen and R¹³ is an optionally substituted C₁₋₆alkyl group or an optionally substituted C₃₋₆cycloalkyl group. The OC(═O)—(CR¹²R¹³)ₘ—C(═O)O group may be referred to as an alkanedioate, and for example may be selected from ethandioate, propandioate, butandioate, pentanedioate, or hexanedioate, each of which may be optionally substituted. In another example, the bidentate ligand may be selected from cyclobutandioate, cyclopentanedioate, or cyclohexanedioate, each of which may be optionally substituted. In another embodiment, R¹² is hydrogen and R¹³ is a C₁₋₆alkyl group or C₁₋₆cycloalkyl group substituted with 1-3 substituents each independently selected from halo, OH, C₁₋₄alkyl, and C₁₋₄alkylCOOH. In one particular embodiment, the bidentate ligand is citrate.

In another embodiment, the bidentate ligand may be OC(O)—CHR¹³—NR¹⁴, wherein R¹³ and R¹⁴ are independently selected from hydrogen, optionally substituted C₁₋₆alkyl, or joined together to form a 4, 5 or 6 membered cycloalkyl or heterocyclic ring. The OC(O)—CHR¹³—NR¹⁴ group may be referred to as an α amino acid, which provides a bidentate ligand as defined herein joined to the platinum atom through its terminal nitrogen atom and terminal oxygen atom. In another embodiment, R¹³ is C₁₋₆alkyl optionally substituted with 1-3 substituents independently selected from halo, OH, COOH, SH, NH(C═NH)NH₂, C(O)NH and optionally substituted monocyclic or bicyclic aryl or hetaryl. For example, the bidentate ligand may be an essential or non-essential α amino acid, such as an α amino acid selected from histidine, isoleucine, leucine, lysine, methionine, phenylalanine, threonine, tryptophan valine, alanine, arginine, asparagines, aspartic acid, cysteine, glutamic acid, glutamine, glycine, ornithine, proline, selenocysteine, serine, or tyrosine.

In another embodiment, the bidentate ligand may be an optionally substituted α, β or δ hydroxycarboxylate, such as a lactate group. In one embodiment, L-L may be selected from OC(O)—(CR¹²R¹³)ₘ—O, which provides a bidentate ligand as defined herein joined to the platinum atom through its terminal oxygen atoms, m is an integer of 1 to 3 and R¹² and R¹³ are each independently selected from hydrogen, halo, OH, COOH, optionally substituted C₁₋₆alkyl, optionally substituted C₃₋₆cycloalkyl and an optionally substituted monocyclic aryl or heteroaryl ring, or when m is 2 or 3 then two groups selected from R¹² and R¹³ from different carbon atoms may be joined together to form an optionally substituted C₃₋₆cycloalkyl or an optionally substituted monocyclic aryl or heteroaryl ring

### Linker

In some embodiments, the platinum-containing moiety P_{M} is attached to the platinum coordinating group P_{C}, which is attached to the surface amino group of the dendrimer through a linking group (L), for example L-P_{C}-P_{M}. The linking group L may be used as a linker for other terminal groups, and may be present with or without terminal groups attached. It will be appreciated that the linking group as described below is also referred to as a “linker”.

In some embodiments, the linker is a group of the formula:

—C(O)—X—C(O)—W—

wherein X is selected from —C₁-C₁₀alkylene-, —(CH₂)_{q}-A-(CH₂)ₜ— and Q;
A is selected from —O—, —S—, —NR₁—, —N⁺(R₁)₂—, —S—S—, —[OCH₂CH₂]ᵣ—O—, —Y—, and —O—Y—O—;
Q is selected from Y or —Z═N—NH—S(O)_{w}—Y—;
Y is selected from cycloalkyl, heterocycloalkyl, aryl and heteroaryl;
Z is selected from —(CH₂)ₓ—C(CH₃)═, —(CH₂)ₓCH═, cycloalkyl and heterocycloalkyl;
R₁ is selected from hydrogen and C₁-C₄ alkyl;
r, q and t are independently selected from 1, 2, 3 and 4;
w is selected from 0, 1 and 2;
x is selected from 1, 2, 3 and 4;
W is absent or is an amino acid residue or a peptide of 1 to 10 amino acid residues; and

In some embodiments, the linker is a group of the formula:

—C(O)—X—C(O)—W-E-

wherein X is selected from —C₁-C₁₀alkylene-, —(CH₂)_{q}-A-(CH₂)ₜ— and Q;
A is selected from —O—, —S—, —NR₁—, —N⁺(R₁)₂—, —S—S—, —[OCH₂CH₂]ᵣ—O—, —Y—, and —O—Y—O—;
Q is selected from Y or —Z═N—NH—S(O)_{w}—Y—;
Y is selected from cycloalkyl, heterocycloalkyl, aryl and heteroaryl;
Z is selected from —(CH₂)ₓ—C(CH₃)═, —(CH₂)ₓCH═, cycloalkyl and heterocycloalkyl;
R₁ is selected from hydrogen and C₁-C₄ alkyl;
r, q and t are independently selected from 1, 2, 3 and 4;
w is selected from 0, 1 and 2;
x is selected from 1, 2, 3 and 4;
W is absent or is an amino acid residue or a peptide of 1 to 10 amino acid residues; and
E is absent or selected from —O—, —S—, —NR₁—, —N⁺(R₁)₂—, —S—S—, —[OCH₂CH₂]ᵣ—O—, —Y—, and —O—Y—O—.

In some embodiments one or more of the following applies:

X is —C₁-C₆-alkylene, —CH₂-A-CH₂—, —CH₂CH₂-A-CH₂CH₂— or heteroaryl;
A is selected from —O—, —S—, —S—S—, —NH—, —N(CH₃)—, —N⁺(CH₃)₂—, —O-1,2-phenyl-O—, —O-1,3-phenyl-O—, —O-1,4-phenyl-O—, —OCH₂CH₂O—, —[OCH₂CH₂]₂—O— and —[OCH₂CH₂]₃—O—;
Y is heteroaryl or aryl, especially thiophenyl, 3,4-propylenedioxythiophenyl or benzene;
Z is —(CH₂)ₓC(CH₃)═, —(CH₂)ₓCH═ and cycloalkyl, especially —CH₂CH₂C(CH₃)═, —CH₂CH₂CH₂C(CH₃)═, —CH₂CH₂CH₂CH═, cyclopentyl and cyclohexyl;
W is a peptide of 2 to 10 amino acid residues;
R₁ is hydrogen, methyl or ethyl, especially hydrogen or methyl, more especially methyl; one of s and t is 1 and the other is 1 or 2, especially were both s and t are 1;
r is 1 or 2, especially 2;
w is 1 or 2, especially 2; and
x is 2 or 3, especially 3.

In particular embodiments the linker comprises a group —C(O)—X—C(O)— which is selected from:

—C(O)—CH₂CH₂—C(O)—, —C(O)—CH₂CH₂CH₂—C(O)—, —C(O)—CH₂OCH₂—C(O)—, —C(O)—CH₂SCH₂—C(O)—, —C(O)CH₂NHCH₂—C(O)—, —C(O)—CH₂N(CH₃)CH₂—C(O)—, —C(O)—CH₂N⁺(CH₃)₂CH₂—C(O)—, —C(O)—CH₂—S—S—CH₂—C(O)—, —C(O)—OCH₂CH₂OCH₂CH₂OC(O)—,

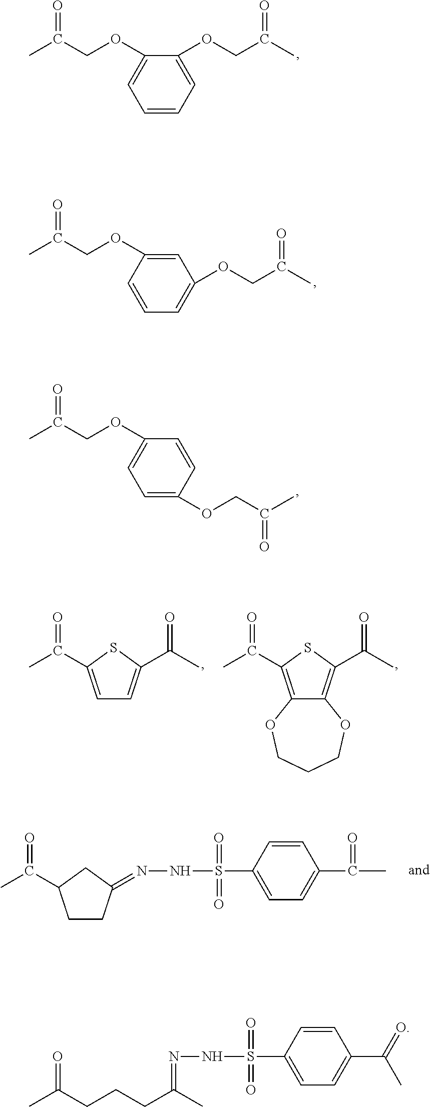

In some embodiments, W is absent and the carbonyl group is connected directly to P_{C}, the platinum coordinating group. In other embodiments, W is present. In particular embodiments, W is a peptide of 2 to 6 amino acid residues. The amino acid residues in the peptide may be any amino acid residues. In particular embodiments, the peptide comprises amino acid residues selected from glycine, leucine, isoleucine, valine, phenylalanine and proline. Suitable peptides include but are not limited to:

SEQ ID NO: 1
-Gly-Gly-Gly-
SEQ ID NO: 2
-Gly-Leu-Phe-Gly-
SEQ ID NO: 3
-Gly-Ile-Leu-Gly-Val-Pro-
SEQ ID NO: 4
-Gly-Phe-Leu-Gly-
SEQ ID NO: 5
-Pro-Val-Gly-Leu-Ile-Gly-.

In some embodiments, the platinum coordinating group, P_{C}, of the linker comprises two functional moieties that coordinate with the platinum atom and a third functional moiety for attachment to W or the group —C(O)—X—C(O)— of the linker. For example, the platinum coordinating group P_{C}, may comprise a bidentate ligand for coordination to the platinum atom of the platinum containing moiety.

In some embodiments, the platinum coordinating group P_{C} comprises two carboxylic acid groups, two nitrogen-containing groups or one nitrogen-containing group and one carboxylic acid group to coordinate with the platinum-containing moiety. In particular embodiments, the platinum coordinating group P_{C} comprises two carboxylic acid groups to coordinate with the platinum-containing moiety.

In some embodiments, the third functional moiety for attachment of the platinum coordinating group P_{C} to the linker is selected from —O—, —S— and —NH—.

In some embodiments, where W is a peptide, one or more further platinum-containing moieties may coordinate to the linker peptide through backbone nitrogen atoms or carbonyl groups and/or side chain groups such as —NH₂, —CO₂H, —OH or —SH.

Exemplary platinum coordinating groups P_{C} for coordinating with the platinum-containing moiety include, but are not limited to:

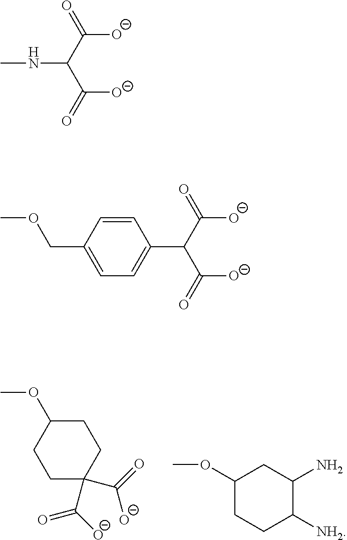

In an embodiment, the platinum coordinating group P_{C} comprises a trans-1,2-diaminocyclohexane for providing a bidentate coordinating group to the platinum atom of the platinum containing moiety.

In some embodiments, the linker (L) is selected from:
*—C(O)CH₂CH₂C(O)-Gly-Gly-Gly-NH—CH(CO₂H)₂,
*—C(O)CH₂CH₂C(O)-Gly-Leu-Phe-Gly-NH—CH(CO₂H)₂,
*—C(O)CH₂CH₂C(O)-Pro-Val-Gly-Leu-Ile-Gly-NH—CH(CO₂H)₂,
*—C(O)CH₂CH₂C(O)—OCH₂—C₆H₄—CH₂CH(CO₂H)₂,
*—C(O)CH₂CH₂C(O)—OCH₂—C₆H₄—CH(CO₂H)₂,
*—C(O)CH₂OCH₂C(O)—OCH₂—C₆H₄—CH₂CH(CO₂H)₂,
*—C(O)CH₂OCH₂C(O)—OCH₂—C₆H₄—CH(CO₂H)₂,
*—C(O)CH₂OCH₂C(O)-Gly-Gly-Gly-NH—CH(CO₂H)₂,
*—C(O)CH₂OCH₂C(O)-Gly-Leu-Phe-Gly-NH—CH(CO₂H)₂,
*—C(O)CH₂OCH₂C(O)-Pro-Val-Gly-Leu-Ile-Gly-NH—CH(CO₂H)₂,
*—C(O)CH₂SCH₂C(O)—OCH₂—C₆H₄—CH₂CH(CO₂H)₂,
*—C(O)CH₂SCH₂C(O)—OCH₂—C₆H₄—CH(CO₂H)₂,
*—C(O)CH₂SCH₂C(O)-Gly-Gly-Gly-NH—CH(CO₂H)₂,
*—C(O)CH₂SCH₂C(O)-Gly-Leu-Phe-Gly-NH—CH(CO₂H)₂,
*—C(O)CH₂SCH₂C(O)-Pro-Val-Gly-Leu-Ile-Gly-NH—CH(CO₂H)₂,
*—C(O)CH₂SCH₂C(O)—OC₆H₉—(CO₂H)₂,
*—C(O)CH₂CH₂C(O)—OC₆H₉—(CO₂H)₂,
*—C(O)CH₂OCH₂C(O)—OC₆H₉—(CO₂H)₂,
*—C(O)CH₂CH₂CH₂C(O)—OC₆H₉—(CO₂H)₂,
*—C(O)CH₂OCH₂C(O)
*—C(O)[(CH₂)ₙX(CH₂)ₚ]ₘC(O), where X is selected from the group consisting of S, SO, SO₂PO, PO₃, NH, NR, alkyl, aryl, and heterocycle; and where n, p and m are independently an integer from 1 to 10,
*—C(O)CH₂CH₂CH₂C(O)
*—C(O)CH₂CH₂C(O)
*—C(O)(CH₂)ₙC(O), where n is an integer from 1 to 10
   wherein * indicates the attachment of the linker to the surface amine group of the dendrimer.

### Four Coordinate Platinum (H) Complexes

In one embodiment, the platinum complex P_{C}-P_{M}, may comprise the following structure, wherein -lig is any monodenetate ligand and -lig-lig- is any bidentate ligand comprising two monodenatate ligands as described herein and the wavy line represents attachment to an optional linker group L as defined herein:

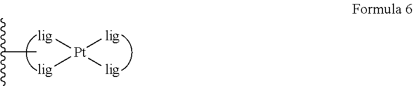

In another embodiment, the platinum complex P_{C}-P_{M}, may comprise the following structure, wherein -lig is any monodenetate ligand and -lig-lig- is any bidentate ligand comprising two monodenatate ligands as described herein and the wavy line represents attachment to an optional linker group L as defined herein:

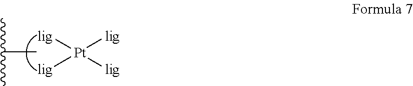

In another embodiment, the platinum complex P_{C}-P_{M}, may comprise the following structure, wherein -lig is a monodentate ligand as described herein and the wavy line represents attachment to an optional linker group L as defined herein:

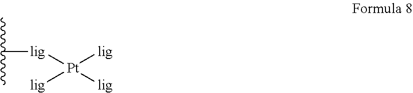

In some embodiments, wherein the platinum coordinating group is chelating group, for example a dicarboxylic acid, a diamino group or an aminocarboxylic acid, the platinum-containing moiety can be chelated to both the functional groups of the platinum coordinating group, for example as a chelating bidentate ligand. For example, the platinum complex (P_{C}-P_{M}) formed by the platinum coordinating group P_{C} and the platinum containing moiety P_{M} may be:

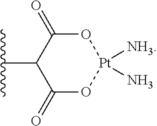

Alternatively, where the platinum coordinating group has three or more functional groups capable of acting as ligands, the platinum-containing moiety may coordinate with any two of the functional groups. For example, the platinum complex (P_{C}-P_{M}) formed by the platinum coordinating group P_{C} and the platinum containing moiety P_{M} may be:

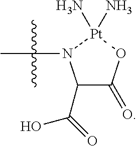

In some embodiments, the platinum-containing moiety may coordinate with only one available functional group on the platinum coordinating group, such as where the platinum coordinating group provides a monodentate ligand. For example, the platinum complex (P_{C}-P_{M}) formed by the platinum coordinating group P_{C} and the platinum containing moiety P_{M} may be as follows:

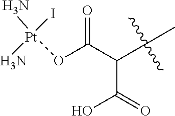

The platinum atom may remain coordinated to a single group on the platinum coordinating group or may coordinate further with a functional group on another location of the linker, or the platinum coordinating group from the same surface amino group.

### Six Coordinate Platinum(IV) Complexes

In further embodiments, the macromolecules can comprise six coordinate platinum(IV) complexes, wherein at least one of the ligands of the platinum complex is provided by a platinum coordinating group as described herein. For example, the platinum coordinating group can provide a single monodentate ligand for the six coordinate platinum(IV) complex. In another embodiment the platinum coordinating group is coordinated to the platinum atom axially.

In an embodiment, the platinum containing moiety P_{M} or platinum complex P_{C}-P_{M}, may comprise the following structure, wherein -lig is any monodenetate ligand and -lig-lig- is any bidentate ligand comprising two monodenatate ligands as described herein and the wavy line represents attachment to a P_{C} or an optional linker group L as defined herein:

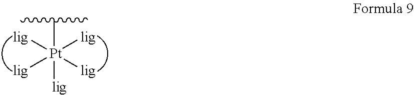

In an embodiment, the platinum containing moiety P_{M} or platinum complex P_{C}-P_{M}, may comprise the following structure, wherein -lig is any monodenetate ligand and -lig-lig- is any bidentate ligand comprising two monodenatate ligands as described herein and the wavy line represents attachment to a P_{C} or an optional linker group L as defined herein:

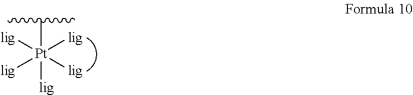

In an embodiment, the platinum containing moiety P_{M} or platinum complex P_{C}-P_{M}, may comprise the following structure, wherein lig is any ligand as described herein and the wavy line represents attachment to a P_{C} or an optional linker group L as defined herein:

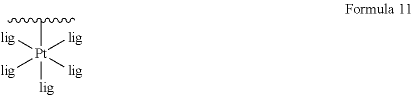

Examples of the macromolecules comprising six coordinate platinum(IV) complexes are provided as follows:

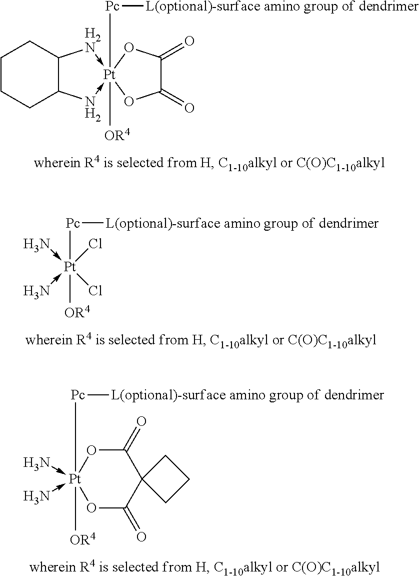

In an embodiment of the above examples, R⁴ is selected from H and C(O)CH₃.

### Terminal Groups

In some embodiments, the macromolecules may comprise further terminal groups selected from the group consisting of pharmacokinetic modifying agents, targeting agents and blocking agents.

In an embodiment, the macromolecules further comprise one or more pharmacokinetic modifying agent (M) wherein the one or more pharmacokinetic modifying agent is attached to the surface amino groups of the dendrimer.

The pharmacokinetic modifying agent (M) may be any molecule or residue thereof that modifies or modulates the pharmacokinetic profile of the platinum-containing moiety or the macromolecule including absorption, distribution, metabolism and/or excretion. In a particular embodiment, the pharmacokinetic modifying agent is an agent selected to prolong the plasma half-life of the platinum-containing moiety or macromolecule, such that the macromolecule has a half life that is greater than the half-life of the platinum-containing oncology drug in a non-dendrimer formulation. Preferably the half life of the macromolecule or composition is at least 2 times and more preferably 10 times greater than the platinum-containing oncology drug in a non-dendrimer formulation.

In some embodiments, the pharmacokinetic modifying agent is polyethylene glycol (PEG), a polyalkyloxazoline such as polyethyloxazoline (PEOX), polyvinylpyrolidone and polypropylene glycol, especially PEG. In other embodiments, the pharmacokinetic modifying agent is a polyether dendrimer. PEG may be monodispersed or polydispersed. MW of PEG is preferably measured as average MW.

In some embodiments, the PEG has a molecular weight of between 220 and 5500 Da. In some embodiments, the PEG has a molecular weight of 220 to 1100 Da, particularly 570 and 1100 Da. In other embodiments, the PEG has a molecular weight of 1000 to 5500 Da, especially 1000 to 2500 Da or 1000 to 2300 Da.

In some embodiments, the macromolecule comprises a plurality of platinum-containing moieties and a plurality of pharmacokinetic modifying agents each independently attached to independent surface amino groups of the dendrimer. The plurality of platinum-containing moieties and pharmacokinetic modifying agents may be present in any ratio. However, in particular embodiments, the ratio of platinum-containing moieties and pharmacokinetic modifying agents is in the range of 4:1 to 1:4, for example about 1:1 to 1:2.

In some embodiments, the macromolecule further comprises one or more targeting agents. The one or more targeting agents may be attached to the surface amino groups of the dendrimer or to a functional group of the core of the dendrimer optionally through a spacer group.

The targeting agent is an agent that binds to a biological target cell, organ or tissue with some selectivity thereby assisting the macromolecule to accumulate at one or more target cell, organ or tissue. The targeting agent may in addition provide a mechanism for the macromolecule to be actively taken into the cell or tissue by receptor mediated endocytosis.

Particular examples include lectins and antibodies and other ligands (including small molecules) for cell surface receptors. The interaction may occur through any type of bonding or association including covalent, ionic and hydrogen bonding, Van der Waals forces.

Suitable targeting agents include those that bind to cell surface receptors. In some embodiments the targeting agent binds to a cancer specific antigen or antigen predominantly expressed on cancer cells.

In some embodiments, the targeting agent is an antibody or antibody fragment such as scFvs and diabodies known to interact with receptors or cellular factors.

The targeting agent may be bound to the dendrimer core directly or preferably through a spacer. For example, the dendrimer core may include at least three functional groups wherein one of the functional groups provides a point of attachment for the targeting agent. The spacer group may be any divalent group capable of binding to both the functional group of the core or surface amino group and the functional group on the targeting agent. The size of the spacer group is preferably sufficient to prevent any steric crowding. Examples of suitable spacer groups include alkylene chains and alkylene chains in which one or more carbon atoms is replaced by a heteroatom selected from —O—, —S—, or NH. The alkylene chain terminates with functional groups suitable for attachment to both the core functional group and the targeting agent. Exemplary spacer groups include X—(CH₂)ₚ—Y, X—(CH₂O)ₚ—CH₂—Y, X—(CH₂CH₂O)ₚ—CH₂CH₂—Y and X—(CH₂CH₂CH₂O)ₚCH₂CH₂CH₂—Y, where X and Y are functional groups for binding with or bound to the core and the targeting agent respectively, and p is an integer from 1 to 100, especially 1 to 50 or 1 to 25.

In some embodiments, the targeting group may be bound to the surface amino groups as a third terminal group. In some embodiments, 1 to 32 targeting groups are bound to the surface.

In some embodiments, the macromolecule further comprises a blocking group that serves to block the reactivity of a surface amino group of the dendrimer. In some embodiments, the blocking group is an acyl group such as a —C₁₋₁₀ acyl group, particularly acetyl.

In some embodiments, some of the surface amino groups of the dendrimer remain unreacted with a linker/chelator/platinum-containing moiety, pharmacokinetic modifying agent, targeting agent or blocking agent. This may be the case in dendrimers with higher generation numbers where steric hinderance may prevent a surface amino group reacting with a further group.

In some embodiments, some of the surface amino groups remain free amino groups.

In some embodiments at least 50% of the total groups bound to the surface amino groups of the dendrimer comprise one of a pharmacokinetic modifying agent or a platinum-containing moiety, and in particular embodiments at least 75% or at least 80% of the groups bound to the surface amino of the dendrimer comprise one of a pharmacokinetic modifying agent or a platinum-containing moiety. In particular embodiments, a platinum-containing moiety is bound to at least about 17.5%, 27%, 32.5%, 37.5%, 42.5%, 45% or 50% of the total number of surface amino groups. Where the dendrimer is a generation 5 (G5) polylysine dendrimer, the total number of the platinum-containing moieties can be more than 10, more than 15, more than 20, or in a further embodiment more than 23. In some embodiments, the pharmacokinetic modifying agent is bound to at least about 15%, 25%, 30%, 35%, 40%, 45% or 50% of the total number of surface amino groups. Where dendrimer is a G5 polylysine dendrimer, the total number of pharmacokinetic modifying agents can be more than 10, more than 15, more than 20, more than 23, and in a particular embodiment more than 28.

### Core and Building Units of Dendrimers

The macromolecule of the invention comprises a dendrimer in which the outermost generation of building units has surface amino groups. The identity of the dendrimer of the macromolecule is not essential, provided it has surface amino groups, although further advantages are provided by particular types of dendrimers. For example, the dendrimer may be a polylysine, polylysine analogue, polyamidoamine (PAMAM), polyethyleneimine (PEI) dendrimer or polyether hydroxylamine (PEHAM) dendrimer.

In some embodiments, the building unit (including subsurface building units D_{B} and surface building units D_{S}) is a polyamine, more preferably a di or tri-amino with a single carboxylic acid. Preferably the molecular weight of the building unit is from 70 Da to 1 kDa. In some embodiments, the building unit is lysine or a lysine analogue.

In some embodiments, the lysine or lysine analogue is selected from a compound of the following formula:

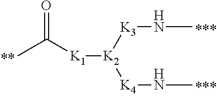

wherein K₁ is absent or is selected from —C₁₋₆ alkylene-, —C₁₋₆ alkyleneNHC(O)—, —C₁₋₆ alkyleneC(O)—, —C₁₋₃ alkylene-O—C₁₋₃ alkylene-, —C₁₋₃ alkylene-O—C₁₋₃ alkyleneNHC(O)— and —C₁₋₃ alkylene-O—C₁₋₃ alkyleneC(O)—;
K₂ is selected from CH or N;
K₃ and K₄ are independently absent or is selected from —C₁₋₆ alkylene- or —C₁₋₃ alkyleneOC₁₋₃ alkylene; provided that when K₃ and/or K₄ are absent, J is CH;
** indicates the linkage between the lysine or lysine analogue and the core of the dendrimer or the previous generation of building units; and
*** indicates the linkage between the lysine or lysine analogue and the subsequent generation of lysine or lysine analogues or forms the surface amino groups of the dendrimer.

In particular embodiments, the lysine or lysine analogue is selected from:

Lysine 1: having the structure:

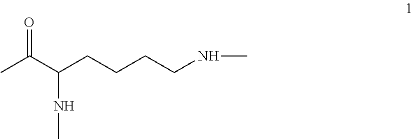

Glycyl-Lysine 2 having the structure:

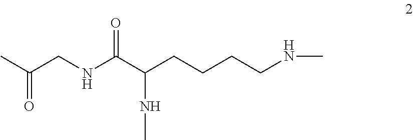 [SMILES: CNCCCCC(NC)C(=O)NCC(C)=O]

Analogue 3, having the structure below, where a is an integer of 1 or 2; b and c are the same or different and are integers of 1 to 4:

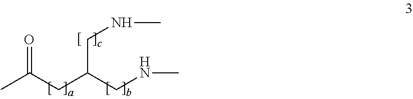

Analogue 4, having the structure below, where a is an integer of 0 to 2; b and c are the same or different and are integers of 2 to 6:

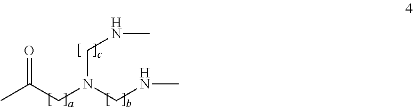

Analogue 5, having the structure below, where a is an integer of 0 to 5; b and c are the same or different and are integers of 1 to 5:

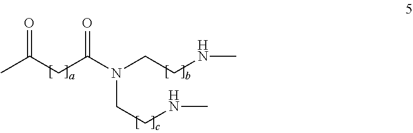

In some embodiments the building unit is an amidoamine building unit with the structure 6:

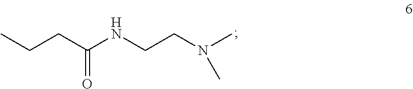

an etherhydroxyamine building unit with the structure 7:

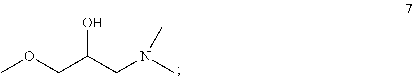

or a propyleneimine building unit with the structure 8:

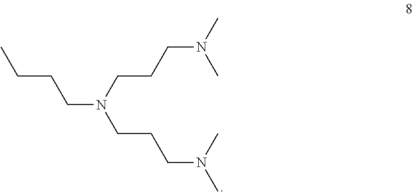 [SMILES: CCCCN(CCCN(C)C)CCCN(C)C]

In a preferred aspect of the invention, the building units are selected from Lysine 1, Glycyl-Lysine 2 or Lysine analogue 5:

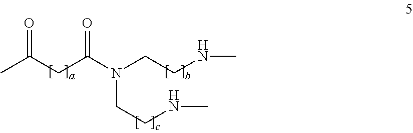

where a is an integer of 0 to 2 or the alkyl link is C—O—C; b and c are the same or different and are integers of 1 to 2; especially where the building units are lysine. In particular embodiments, the molecular weight of the building unit is from 70 Da to 1 kDa.

In some embodiments, the core is a monoamine compound, diamine compound, triamine compound, tetraamine compound or pentamine compound, one or more of the amine groups having a dendron comprising building units attached thereto.

Suitable cores include benzhydrylamine (BHA), a benzhydrylamide of lysine (BHALys) or a lysine analogue, or:

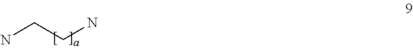

where a is an integer of 1 to 9, preferably 1 to 5;

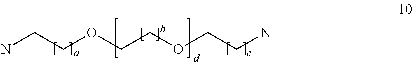

where a, b and c, which may be the same or different, and are integers of 1-5, and d is an integer from 0-100, preferably 1-30;

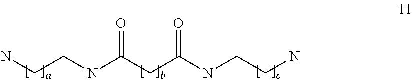

where a and c, which may be the same or different, are integers of 1 to 6 and where b is an integer from 0 to 6;

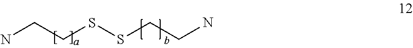

where a and b are the same or different and are integers of 1 to 5, especially 1 to 3, more especially 1;
a triamine compound selected from:

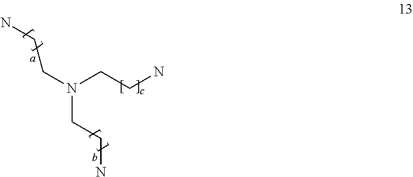

where a, b and c, which may be the same or different, are integers of 1 to 6;

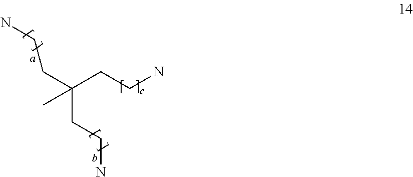

where a, b and c, which may be the same or different, are integers of 0 to 6;

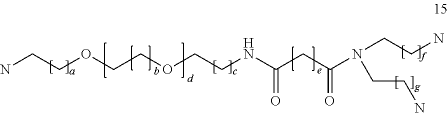

wherein a, b and c, which may be the same or different, are integers of 1 to 5, d is an integer from 1 to 100, preferably 1 to 30, e is an integer from 0 to 5 and f and g are the same or different and are integers from 1 to 5;
or a tetraamine compound selected from

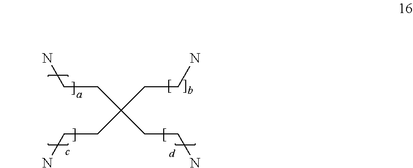

where a, b, c and d, which may be the same or different, are integers of 0 to 6;

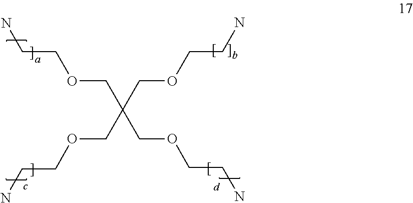

where a, b, c and d, which may be the same or different, are integers of 1 to 6;
and furthermore, the alkyl chain moieties (eg: —C—C—C—) of the building units may be understood to include alkoxy fragments such as C—O—C or C—C—O—C—C where one or more non-adjacent carbon atom is replaced with an oxygen atom, provided that such a substitution does not form a O—C—X group where X is O or N.

In some embodiments, the core has at least two amino functional groups, one of which has attached a targeting moiety either directly or through a spacer group. At least one of the remaining functional groups of the core having a dendron attached as described in WO 2008/017125.

In particular embodiments, the core is BHA or BHALys or NEOEOEN[SuN(PN)₂], where N is an amino group, E is an ethyl group, Su is a succinate group and PN is a propylamino group.

In some embodiments, the dendrimer has 1 to 5 dendrons attached to the core, especially 2 to 4 dendrons, more especially 2 or 3 dendrons.

In some embodiments, the dendrimer has 1 to 8 generations of building units, especially 2 to 7 generations, 3 to 6 generations, more especially 4 to 6 generations.

In some embodiments, the macromolecules of the invention comprise 1% to 20% w/w platinum, especially 2.5% to 10% w/w, as measured by ICP-OES analysis.

The macromolecule of the invention may be nanoparticle having a particulate diameter of below 1000 nm, for example, between 5 and 1000 nm, especially 5 and 500 nm, more especially 5 to 400 nm, such as 5 to 50 nm, especially between 5 and 20 nm. In particular embodiments, the composition contains macromolecules with a mean size of between 5 and 20 nm. In some embodiments, the macromolecule has a molecular weight of at least 30 kDa, for example, 40 to 150 kDa or 40 to 300 kDa.

In some embodiments, the macromolecules of the invention have a particle size that is suitable for taking advantage of the Enhanced Permeability and Retention Effect (EPR effect) in tumors. Tumor vasculature is abnormal, making it permeable to particles of a size that then collect in tumor tissue. Furthermore, tumor tissues lack effective lymphatic drainage and therefore macromolecules are retained there.

### Processes for Preparing Macromolecules

Methods of making dendrimers are known in the art. For example, the dendrimers of the macromolecule may be made by a divergent method or a convergent method or a mixture thereof.

In the divergent method each generation of building units is sequentially added to the core or an earlier generation. The surface generation having one or both of the surface amino groups protected. If one of the amino groups is protected, the free amino group is reacted with one of the linker, the linker-platinum-containing moiety or optionally one of the other terminal groups. If more than one type of group is to be bound to the surface amino groups and both amino groups of a building unit are protected, they are protected with different protecting groups, one of which may be removed without removal of the other. One of the amino protecting groups is removed and reacted with one of the linker, the linker-platinum-containing moiety or one of the other terminal groups. Once the first type of group has been attached to the dendrimer, the other amino protecting group is removed and the other type of terminal group is added. These groups are attached to the surface amino groups by amide formation as known in the art.

In the convergent method, each generation of building units is built up on the previous generation to form a dendron. The linker/platinum-containing moieties and optionally other terminal groups may be attached to the surface amino groups as described above before or after attachment of the dendron to the core.

In a mixed approach, each generation of building units is added to the core or a previous generation of building units to form a dendrimer scaffold. However, the final generation of building units are functionalised with the linker/platinum-containing moiety and optionally other terminal groups before they are added to the dendrimer. The functionalised building units are then added to the dendrimer scaffold.

The platinum-containing moiety is complexed with the platinum coordinating group of the linker as known in the art. For example, a platinum complex containing two reactive ligands, such as water or chloride, in a cis orientation, is reacted with the platinum coordinating group of the linker at a mildly basic pH, for example, 7.5.

In the case where a targeting agent is attached to the core, a functional group on the core may be protected during formation of the dendrimer then deprotected and reacted with the targeting agent, the spacer group or the targeting agent-spacer group. Alternatively, the core may be reacted with the spacer group or targeting agent-spacer group before the formation of the dendrimer.

Suitable protecting groups, methods for their introduction and removal are described in Greene & Wuts, Protecting Groups in Organic Synthesis, Third Edition, 1999.

### Compositions

While it is possible that the macromolecules of the invention may be administered as a neat chemical, in particular embodiments, the macromolecule is presented as a pharmaceutical composition.

The invention provides pharmaceutical formulations or compositions, both for veterinary and for human medical use, which comprise one or more macromolecules of the invention or a pharmaceutically acceptable salt thereof, with one or more pharmaceutically acceptable carriers, and optionally any other therapeutic ingredients, stabilisers, or the like. The carrier(s) must be pharmaceutically acceptable in the sense of being compatible with the other ingredients of the formulation and not unduly deleterious to the recipient thereof. The compositions of the invention may also include polymeric excipients/additives or carriers, e.g., polyvinylpyrrolidones, derivatised celluloses such as hydroxymethylcellulose, hydroxyethylcellulose, and hydroxypropylmethylcellulose, Ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, pectin mannitol and trehalose. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, EGTA). Other pharmaceutical excipients and/or additives suitable for use in the compositions according to the invention are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

The macromolecule may also be formulated in the presence of an appropriate albumin protein such as human serum albumin. Albumin carries nutrients around the body and may bind to the macromolecule and carry it to its site of action.

The macromolecules of the invention may be formulated in compositions including those suitable for parenteral (including intraperitoneal, intravenous, subcutaneous, or intramuscular injection) or pulmonary (including intratracheal or intranasal) administration. The compositions may conveniently be presented in unit dosage form and may be prepared by any of the methods well known in the art of pharmacy. All methods include the step of bringing the macromolecule into association with a carrier that constitutes one or more accessory ingredients. In general, the compositions are prepared by bringing the macromolecule into association with a liquid carrier to form a solution or a suspension. In general, for solid formulations intended for intravenous administration, particles will typically range from about 1 nm to about 10 microns in diameter. The composition may contain macromolecule of the invention that are nanoparticulate having a particulate diameter of below 1000 nm, for example, between 5 and 1000 nm, especially 5 and 500 nm, more especially 5 to 400 nm, such as 5 to 50 nm and especially between 5 and 20 nm. In particular embodiments, the composition contains macromolecules with a mean size of between 5 and 20 nm. In some embodiments, the macromolecule is polydispersed in the composition, with a polydispersity index (PDI) of between 1.01 and 1.8, especially between 1.01 and 1.5, and more especially between 1.01 and 1.2. In particular embodiments, the macromolecule is monodispersed in the composition. Particularly preferred are sterile, lyophilized compositions that are reconstituted in an aqueous vehicle prior to injection.

Formulations suitable for parenteral administration conveniently comprise a sterile aqueous preparation of the macromolecule, which can be formulated to be isotonic with the blood of the recipient.

### Methods of Use

The macromolecule of the invention may be used to treat or prevent any disease, disorder or symptom that a platinum-containing oncology drug can be used to treat or prevent.

In some embodiments, the macromolecule is used in a method of treating or preventing cancer, or suppressing the growth of a tumor.

In some embodiments, the cancer is a blood borne cancer such as leukaemia or lymphoma. In other embodiments, the cancer is a solid tumor. The solid tumor may be a primary or a metastatic tumor. Exemplary solid tumors include tumors of the breast, lung especially non-small cell lung cancer and small cell lung cancer, colon, stomach, kidney, bladder, brain, pancreas, head and neck especially squamous cell carcinoma of the head and neck, thyroid, ovary, testes, liver, cervix, melanoma, prostate especially androgen-independent (hormone refractory) prostate cancer, neuroblastoma and gastric adenocarcinoma including adenocarcinoma of the gastroesophageal junction. In particular embodiments, the tumor is a platinum responsive tumor.

Oncology drugs often have significant side effects that are due to off-target toxicity.

In another aspect of the invention, there is provided a method of reducing the side effects of a platinum-containing oncology drug or the side-effects relating to the formulation of a platinum-containing oncology drug comprising administering an effective amount of the macromolecule of the present invention to a subject.

In some embodiments, the side effects reduced are hematologic toxicity, neurological toxicity, cardiotoxicity, hepatotoxicity, nephrotoxicity, pulmonary toxicity, ototoxicity and encephalotoxicity. More particularly there may be a reduction in infections, neutropenia, anemia, febrile neutropenia, hypersensitivity, thrombocytopenia, myelotoxicity, myelosuppression, neuropathy, dysgeusia, dyspnea, constipation, anorexia, nail disorders, fluid retention, asthenia, pain, nausea, diarrhea, vomiting, fatigue, non-specific neuro cognitive problems, vertigo, encephalopathy, ototoxicity, mucositis, alopecia, skin reactions and myalgia. Neuropathic side effects are varied and include acute peripheral, reversible sensitivity to cold and numbness, foot/leg and/or hand/arm pain, often with deficits in proprioception and sensory impairment, as well as pharyngolaryngeal dysesthesia. Side effects may be chronic or acute. Chronic neuropathies include peripheral nerve damage that can take up to a year to recover.

In the present invention, the macromolecule comprising the platinum-containing moiety reduces off target side effects associated with the platinum-containing oncology drug as it passively accumulates at the tumor site or is directed to the tumor site by an appropriate targeting agent and release of the drug from the dendrimer is controlled.

Furthermore, the macromolecules of the present invention need not be administered by prolonged infusion. In some embodiments, they may be administered by fast-infusion, for example, in less than 3 hours, including 2.5 hours, 2 hours, 1.5 hours, 1 hour or 30 minutes. In some embodiments, the macromolecule or formulation of macromolecule may be administered as a bolus, for example, in 5 seconds to 5 minutes.

The macromolecules of the present invention may also allow the dose of the platinum-containing moiety to be increased compared to that delivered by a platinum-containing oncology drug. In another aspect of the invention there is provided a method of increasing the dose of a platinum-containing moiety delivered comprising administering the macromolecule of the present invention comprising a platinum-containing moiety. In particular embodiments, the maximum tolerated dose of platinum-containing moiety is increased at least two fold compared to the platinum-containing oncology drug.

An “effective amount” means an amount necessary at least partly to attain the desired response, or to delay the onset or inhibit progression or halt altogether, the onset or progression of a particular condition being treated. The amount varies depending upon the disease being treated, such as cancer, the number of platinum ions included in the macromolecule, the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials. An effective amount of macromolecule expressed in Platinum equivalents, in relation to a human patient, for example, may lie in the range of about 0.1 mg to 100 mg per kg of body weight per dosage. In another embodiment, the dosage is in the range of 1 mg to 10 mg per kg of body weight per dosage, such as up to 5 mg per kg of body weight per dosage. Dosage regimens may be adjusted to provide the optimum therapeutic response. For example, several divided doses may be administered daily, weekly, monthly or other suitable time intervals, or the dose may be proportionally reduced as indicated by the exigencies of the situation.

In some embodiments the macromolecule is administered intravenously, intraperitonealy, intrathecally, intrapulmonary, intranasally, by inhalation, intratracheally or intraarterially.

In some embodiments, the subject is a mammal, especially a human, domestic companion animal such as a cat or dog, a farmed animal such as pigs, cattle, sheep and the like, a laboratory animal such as mice, rats, rabbits, monkeys and the like, or a captive wild animal such as those kept in zoos. In particular embodiments, the subject is a human.

In another aspect of the invention there is provided the use of a macromolecule of the invention in the manufacture of a medicament for preventing, treating or suppressing the growth of cancer or reducing the side effects of a platinum-containing oncology drug.

### Further Embodiments of Macromolecules

In another aspect, there is provided a macromolecule comprising:
a. a dendrimer comprising a core and at least one generation of building units having surface amino groups; and
b. one or more platinum-containing moiety;
wherein the one or more platinum-containing moiety is attached to the surface amino group of the dendrimer through a chelating group that chelates the platinum-containing moiety; or a pharmaceutically acceptable salt thereof.

The macromolecules according to the above aspect can further comprise one or more pharmacokinetic modifying agents, wherein the one or more pharmacokinetic modifying agent is attached to the surface amino groups of the dendrimer. The macromolecules can comprise a plurality of platinum-containing moieties and a plurality of pharmacokinetic modifying agents attached to the surface amino groups of the dendrimer. The ratio of platinum-containing moieties and pharmacokinetic modifying agents can be in the range of 1:4 to 4:1. The platinum-containing moiety can comprise a platinum ion in the +2 oxidation state.

In further embodiments of the above aspect, the chelating group and the platinum-containing moiety can form a square planar complex. The platinum-containing moiety can comprise two inert nitrogen atoms that form part of one or two molecules, coordinated to the platinum ion in a cis orientation. The platinum-containing moiety can be selected from:

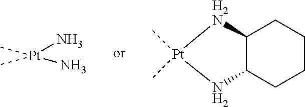

wherein the dashed lines indicate Pt coordination with the chelating group.

In further embodiments of the above aspect, the platinum-containing moiety can be attached to the surface amino group of the dendrimer through a linker that comprises the chelating group. The linker can comprise a group of the formula:

—C(O)—X—C(O)—W-D-

wherein X is selected from —C₁-C₁₀alkylene-, —(CH₂)ₛ-A-(CH₂)ₜ— and Q;
A is selected from —O—, —S—, —NR₁—, —N⁺(R₁)₂, —S—S—, —[OCH₂CH₂]ᵣ—O—, —Y—, and —O—Y—O—;
Q is selected from Y or —Z═N—NH—S(O)_{w}—Y—;
Y is selected from cycloalkyl, heterocycloalkyl, aryl and heteroaryl;
Z is selected from —(CH₂)ₓ—C(CH₃)═, —(CH₂)ₓCH═, cycloalkyl and heterocycloalkyl;
R₁ is selected from hydrogen and C₁-C₄ alkyl;
r, s and t are independently selected from 1, 2, 3 and 4;
w is selected from 0, 1 and 2;
x is selected from 1, 2, 3 and 4;
W is absent or is an amino acid residue or a peptide of 2 to 10 amino acid residues; and
D is a chelating group for chelating, at least partially, with the platinum-containing moiety.

In a further embodiment of the above aspect, one —C(O)— group of the linker forms an amide bond with the surface amino group of the dendrimer and the other —C(O)— group is attached to the chelating group -D-, optionally through a peptide, W. In another embodiment, X can be selected from —C₁₋₆alkylene-, —CH₂-A-CH₂— and —CH₂CH₂-A-CH₂CH₂—. X can be selected from —CH₂CH₂—, —CH₂CH₂CH₂—, —CH₂OCH₂— and —CH₂SCH₂—. In yet a further embodiment, W is a peptide of 2 to 10 amino acid residues in length. The peptide can be selected to be 3 to 6 amino acid residues in length. The peptide can be selected from -GGG-, -GFLG-, GLFG-, -GILGVP- and PVGLIG-.

In a further embodiment of the above aspect, the chelating group comprises two functional moieties that coordinate with a platinum atom and a third functional moiety for attachment to the linker. The chelating group can comprise two carboxylic acid groups, two nitrogen-containing groups or one nitrogen-containing group and one carboxylic acid group. The chelating group can comprise two carboxylic acid groups. The third functional moiety of the chelating group can be selected from —O—, —S—, —CO— and —NH—. In a particular embodiment, the chelating group can be selected from:

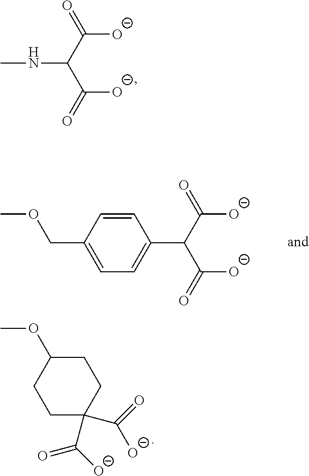

The invention will now be described with reference to the following Examples which illustrate some particular aspects of the present invention. However, it is to be understood that the particularity of the following description of the invention is not to supersede the generality of the preceding description of the invention.

### Abbreviations

| | |
|---|---|
| ACN | Acetonitrile |
| LCMS | Liquid chromatography mass spectrometry |
| BHAlysine | Benzhydrylamide lysine |
| Lys | Lysine |
| Boc | benzyloxycarbonyl |
| PBS | Phosphate buffered saline |
| DIPEA | diisopropylethylamine |
| PEG | Polyethylene glycol |
| PyBop | Benzotriazol-1-yl-oxytri-pyrrolidinophosphonium |
| | hexafluorophosphate |
| SEC | Size exclusion chromatography |
| SRB | Sulforhodamine B |
| TFA | Trifluoroacetic acid |
| NMM | N-methylmorpholine |
| HPLC | High Performance Liquid Chromatography |
| DACH | Diaminocyclohexane |
| DMF | Dimethylformamide |

### EXAMPLES

The dendrimers represented in the examples below include reference to the core and the building units in the outermost generation of the dendrimer. The 1^{st} to subsurface generations are not depicted. The dendrimer BHALys[Lys]₃₂ is representative of a 5 generation dendrimer having the formula BHALys[Lys]₂[Lys]₄[Lys]₈[Lys]₁₆[Lys]₃₂, the 64 surface amino groups being available to bind to terminal groups.

Preparation of the dendrimer scaffolds BHALys[Lys]₃₂[α-NH₂.TFA]₃₂[ε-PEG₁₁₀₀]₃₂ and BHALys[Lys]₃₂[α-NH₂.TFA]₃₂[ε-PEG₂₂₀₀]₃₂ can be found in Kaminskas et al., J. Control. Release (2011) doi 10.1016/j.jconrel.2011.02.005. Preparation of cis-diaqua-1R,2R-DACH-Platinum(II) dinitrate and cis-diamminediaquaPlatinum(II) dinitrate can be found in U.S. Pat. No. 7,166,733.

### General Procedures

### General Procedure A. Preparation of Peptide Linkers

Step 1: To a solution of 1-ethyl-3-(3-dimethylaminopropyl)carbodiimide (1.2 equivalents), diisopropylethyl amine (2.7 equivalents), N-tert-butyl protected tripeptide/tetrapeptide/hexapeptide (1.0 equivalent) and 1-hydroxy-benzotriazole (1.2 equivalents) in dimethylformamide (3-30 mL was added a solution of diethyl aminomalonate hydrochloride (0.92-17.2 mmol) and diisopropylethyl amine (1.0 equivalent) in dimethylformamide (2-10 mL) under nitrogen atmosphere. The pale yellow solution was left to stir at room temperature for 15 hours. Volatiles were evaporated over rotary evaporator and the residue obtained was partitioned between water and ethyl acetate. The aqueous layer was extracted with ethyl acetate (4×100 mL). The combined organic extract was washed with 1N hydrochloric acid, saturated sodium bicarbonate solution and brine, filtered through a cotton plug and evaporated to give the crude product which was chromatographed over SiO₂ flash column using dichloromethane and methanol and the pure desired product (53%-66%) was obtained.

Step 2: To a solution of tert-butyl protected peptido amino diethylmalonate (0.57-10.5 mmol) from step 1 in dichloromethane (15-100 mL) was added trifluoroacetic acid (10-20 equivalents). The reaction mixture was stirred at room temperature under nitrogen atmosphere for 15 hours. Volatiles were evaporated over rotary evaporator. The white solid obtained was triturated with petroleum ether/diethyl ether a couple of times to give product as an off-white fluffy solid (94%-quant.).

Step 3: To a solution of peptido amino diethylmalonate trifluoroacetate (0.57-11.7 mmol) from step 2 in dimethylformamide (5-20 mL) was added diisopropylethyl amine (1.2 equivalents) and the mixture stirred for 20 min. To this solution was added another solution of 1-ethyl-3-(3-dimethylaminopropyl)carbodiimide (1.2 equivalents) in dimethylformamide (5-20 mL) and diisopropylethyl amine (2.5 equivalents) in dimethylformamide (5-20 mL). The resulting reaction mixture was stirred under nitrogen for 15 hours at room temperature, then filtered through a G3 sintered funnel if a precipitate was formed. The filtrate was extracted with ethyl acetate (3×100 mL), dried over MgSO₄, filtered and evaporated to give the white solid which was combined with the precipitate obtained earlier. Pure compound (43%-65%) could be obtained either after trituration of the solid compound with petroleum ether or after column chromatography over SiO₂ flash column using dichloromethane and methanol as the eluent.

Step 4: To a suspension of tert-butyl protected succinyl peptido amino diethylmalonate (0.35-4.7 mmol) from step 3 in dichloromethane (15-30 mL) was added trifluoroacetic acid (10-25 equivalents). The mixture was stirred at room temperature for 15 hours. Volatiles were evaporated over rotary evaporator. The sticky solid obtained was triturated with petroleum ether and diethyl ether to give the tert-butyl deprotected product as white solid (91%-quant.).

### General Procedure B. Preparation of Ester Based Linkers

Step 1: To a solution of di-tert-butyl 2-(4-formylphenyl) malonate (3.12 mmol) in methanol (25 mL) was added sodium borohydride (1.0 equivalent) under nitrogen atmosphere and the mixture was left to stir at room temperature for 2.5 hours. Cold water (10 mL) was added dropwise to quench excess sodium borohydride. Volatiles were evaporated over rotary evaporator. The obtained residue was partitioned between dichloromethane and 0.1 M aq. hydrochloric acid. The acidic aqueous layer was extracted with dichloromethane (3×20 mL). The combined organic extracts were washed with saturated sodium bicarbonate solution and brine, then dried over sodium sulphate, filtered and evaporated to give white solid (quant.).

Step 2: To a solution of di-tert-butyl 2-(4-hydroxymethylphenyl) malonate (0.16-0.62 mmol) from step 1 in dichloromethane (2.5-10 mL) was added diglycolic anhydride or thiodiglycolic anhydride (1.0 equivalent) followed by triethylamine (2.9 equivalents). The reaction mixture was stirred at room temperature under nitrogen atmosphere for 15 hours. The reaction mixture was washed with phosphate buffer at pH=3. The organic phase was dried over magnesium sulphate, filtered and evaporated to give desired product (quant.).

General Procedure C. Loading Dendrimer with Linker.

Step 1: To a magnetically stirred mixture of BHALys[Lys]₃₂[α-NH₂.TFA]₃₂[ε-PEG_{1100/2200}]₃₂ (0.5-1.0 μmol) and DIPEA or NMM (4.0 equivalents per amine) in DMF at room temperature was added linker (1.2 equivalents per amine group) and PyBOP (1.2 equivalents per amine group). After 15 hours at room temperature, the volatiles were removed and the residue purified either by ultrafiltration or by SEC (Sephadex™, LH20, MeOH). The appropriate fractions, as judged by HPLC, were combined and concentrated to provide the desired material.

Step 2: To a slightly turbid solution of BHALys[Lys]₃₂[α-NH-linker]₃₂[ε-PEG_{1100/2200}]₃₂ (0.006 mmol) in water (15 mL) was added 1M aqueous sodium hydroxide solution (0.5 mmol). The reaction mixture was left to stir for 15 hours at room temperature. CG-Amberlite™ cation exchange resin-H+ form (approx. 90 mg) was added with constant stirring. When the pH was stabilised at ˜7.0, the reaction mixture was filtered through 0.2 μM filter. Deionised water (150 mL) was added and the diluted product ultrafiltered through a 10 kDa Omega Centramate™ membrane. The retentate was freeze dried to give the desired product as a white solid.

General Procedure D. Coordinating Dendrimer-Linker with Platinum Drug.

To a solution of BHALys[Lys]₃₂[α-NH-linker(diacid)]₃₂[ε-PEG_{1100/2200}]₃₂-linker (0.005 mmol) in water (10 mL) was added 1M aqueous sodium hydroxide solution (˜0.2 mL) to bring the pH to 7.5. cis-Diaqua-1R,2R DACH/diammine Platinum(II) dinitrate solution in water (24.2 mM) was added. The pH of the solution was adjusted to 5.5 and the reaction mixture was stirred for 2 hours at room temperature before pH was raised to 7.5 using 1M aqueous sodium hydroxide solution. The reaction mixture was then heated at 38° C. for 15 hours, during this time pH was maintained at 7.5. The reaction mixture was cooled to room temperature and filtered through 0.22 μM filter and ultrafiltered through 10 kDa Omega Centramate™ membrane (first ultrafiltration). This retentate on freeze drying would give O,O′-Platinum coordinated dendrimer. To obtain N,O′-Platinum coordination, the retentate (500 mL) was treated with NaCl (2.92 g, 50 mmol), Na₂HPO₄.2H₂O (8.17 g, 30.5 mmol), NaH₂PO₄ (0.965 g, 7.0 mmol) to make 100 mM in Cl⁻ and 75 mM in PO₄²⁻. After dissolution of all the salts, filtered through 0.22 μM filter and the filtrate kept at 38° C. for 15 hours. Cooled to room temperature, filtered through 0.22 μM filter and then ultrafiltered through 10 kDa Omega Centramate™ membrane (second ultrafiltration). The retentate was collected and freeze dried to yield platinum coordinated product as a brown solid.

### LC-MS

LC-MS was recorded either on Waters XBridge C8 column with gradient 40-90% ACN/H₂O (1-7 min), 90% ACN (7-9 min), 90-40% ACN (9-11 min), 40% ACN (11-15 min), 0.1% HCOOH or Waters XBridge C18 column with gradient i): 5-60% ACN/H₂O (1-10 min), 60% ACN (10-11 min), 60-5% ACN (11-13 min), 5% ACN (13-15 min), 0.1% HCOOH or gradient ii); 0-10% ACN/H₂O (1-10 min), 10% ACN (10-12 min), 10-60% ACN/H₂O (12-12.10 min), 60% ACN/H₂O (12.10-14 min), 60-0% ACN (14-14.10 min), 0% ACN (14.10-18 min), 0.1% HCOOH

### HPLC

HPLC was recorded on XBridge C8 column with gradient 5-80% ACN/H₂O (1-7 min), 80% ACN (7-9 min), 80-5% ACN (9-11 min), 5% ACN 11-15 min), 10 mM trifluoroacetic acid) and UV detection at 214 nm.

### General Procedure for Determining Molecular Weights

Molecular weights were estimated via quantification and then addition of each of the dendrimers individual components, namely Lysine, PEG, linker and Pt. Quantification of each component on the dendrimer was achieved sequentially as the material progressed through a synthetic sequence by either mass spectrometery, 1H NMR or Pt content as measured by ICP-OES as well as by comparison to material from the preceding step. A G5 polylysine dendrimer with a BHALys core and 62 lysines has a molecular weight of 8.3 kDa. This compound has been fully characterised in the past using a number of analytical techniques and exists as predominantly a single molecular species. The lysine 1H NMR signals from this core dendrimer form key reference points for subsequent NMR analysis. The PEGylation reagents are purchased with a certificate of analysis (CoA) which outlines the average molecular weight and polymer distribution for the product. This information is used to determine the number of ethylene protons that are expected in a 1H NMR spectrum (δ 3.4-3.9 ppm) per PEG molecule. The extent of pegylation of a dendrimer is estimated via integration of the PEG protons in the NMR spectrum (δ 3.4-3.9 ppm) compared to the number expected for a single PEG reagent. Lysine protons form a suitable internal reference check. Similarly, the number of linker groups is estimated via integration of characteristic 1H NMR peaks compared to either the PEG or lysine signals e.g. for the GLFG linker, the phenylalanine protons in the aromatic region (δ 7.1-7.4 ppm) form a characteristic handle that can be compared to either the PEG ethylene protons or the lysine proton signals. Finally, ICP-OES provides the % Pt which in turn indicates the amount of chelator present e.g. for Pt(DACH) in the construct a measure of 8.4% w/w Pt equates to 13.3% w/w Pt-DACH.

### Platinum Determination by ICP-OES

Approx. 1.5 mL of 2% HCl was added to the platinum-dendrimer conjugate (for example: 5.55 mg) to dissolve. The solution was diluted with 2% HCl to 10 mL. This solution was further diluted 1 in 5 with 2% HCl. The sample solution was then analysed in duplicate by ICP-OES against a 5 point calibration curve for platinum content yields the platinum concentration, expressed as a percentage of platinum on a w/w basis for a known mass of platinum-dendrimer conjugate.

### Platinum Equivalents

The molecular weights of moieties containing platinum differ considerably yet the platinum is considered the active species. In order to compare such moieties, a measure of platinum content called “platinum equivalents” is used. “Platinum equivalents” measures only the mass of the platinum (as calculated above) and the rest of the molecular mass of the construct is not taken into account. e.g. 10 mg of oxaliplatin (molecular weight is 397.28) contains 4.9 mg of platinum (MW is 195.08), therefore a dose of 10 mg/kg oxaliplatin is expressed as 4.9 mg/kg platinum equivalents.

### Example 1

### Preparation of BHALys[Lys]₃₂[α-Succ-Gly-Gly-Gly-amino malonic acid-Pt-1R,2R-(DACH)]₃₂[ε-PEG₂₂₀₀]₃₂ (Compound 1)

### (a) Preparation of tert-BuO-Gly-Gly-Gly-amino diethylmalonate

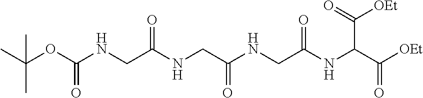 [SMILES: CCOC(=O)C(NC(=O)CNC(=O)CNC(=O)CNC(=O)OC(C)(C)C)C(=O)OCC]

Prepared using Procedure A, step 1 above, using tert-butyl protected triglycine (5.0 g, 17.2 mmol) SiO₂ flash chromatography provided 4.7 g (61%) of product as a white solid. LCMS (C18 column): Rf (min)=9.40. ESI (+ve) observed [M+H]⁺=447. Calculated for C₁₈H₃₀N₄O₉=446.45 Da. ¹H-nmr (300 MHz, CD₃OD) δ (ppm): 1.30 (t, J=6 Hz, 6H), 1.46 (s, 9H), 3.76 (s, 2H), 3.94 (s, 2H), 4.00 (s, 2H), 4.21-4.31 (m, 4H), 5.17 (s, 1H).

### (b) Preparation of H₂N-Gly-Gly-Gly-amino diethylmalonate trifluoroacetate

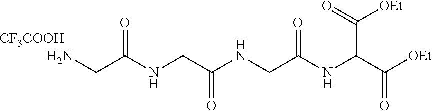 [SMILES: "CF3COOH" ; CCOC(=O)C(NC(=O)CNC(=O)CNC(=O)CN)C(=O)OCC]

Prepared using Procedure A, step 2 above, using tert-butyl protected triglycine amino diethylmalonate (4.7 g, 10.5 mmol). Desired product obtained as white solid (4.8 g, quant.). LCMS (C18 column), Rf (min)=5.49. ESI (+ve) observed [M+H]⁺=347. Calculated for C₁₃H₂₂N₄O₇=346.15 Da. ¹H-nmr (300 MHz, CD₃OD) δ (ppm): 1.30 (t, J=6 Hz, 6H), 3.76 (s, 2H), 4.01 (s, 2H), 4.02 (s, 2H), 4.21-4.31 (m, 4H), 5.18 (s, 1H).

### (c) Preparation of tert-BuO-Succ-Gly-Gly-Gly-amino diethylmalonate

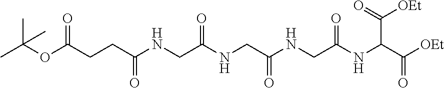 [SMILES: CCOC(=O)C(NC(=O)CNC(=O)CNC(=O)CNC(=O)CCC(=O)OC(C)(C)C)C(=O)OCC]

Prepared using Procedure A, step 3 above, using triglycine amino diethylmalonate (5.4 g, 11.7 mmol). Desired product obtained as white solid (2.5 g, 43%). LCMS (C18 column); Rf (min)=9.67. ESI (+ve) observed [M+H]⁺=503. Calculated for C₂₁H₃₄N₄O₁₀=502.52 Da. ¹H-nmr (300 MHz, DMSO-d₆) δ (ppm): 1.20 (t, J=6 Hz, 6H), 1.38 (s, 9H), 2.35-2.43 (In, 4H), 3.70-3.73 (m, 4H), 3.83 (d, J=4 Hz, 2H), 4.12-4.23 (m, 4H), 5.08 (d, J=9 Hz, 1H), 8.08 (br t, J=6 Hz, 2H), 8.14 (br t, J=6 Hz, 1H), 8.76 (d, J=9 Hz, 1H).

### (d) Preparation of HO-Succ-Gly-Gly-Gly-amino diethylmalonate

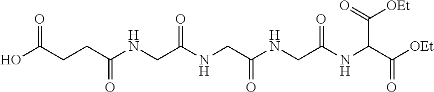 [SMILES: CCOC(=O)C(NC(=O)CNC(=O)CNC(=O)CNC(=O)CCC(=O)O)C(=O)OCC]

Prepared using Procedure A, step 4 above, using tert-butyl protected succinyl-triglycine amino diethylmalonate (2.3 g, 4.7 mmol). Desired product obtained as white solid (1.9 g, 91%). LCMS (C18 Column): Rf (min)=6.74. ESI (+ve) observed [M+H]⁺=447. Calculated for C₁₇H₂₆N₄O₁₀=446.41 Da. ¹H-nmr (300 MHz, DMSO-d₆) δ (ppm): 1.20 (t, J=6 Hz, 6H), 2.35-2.47 (tn, 4H), 3.70-3.73 (m, 4H), 3.83 (d, J=6 Hz, 2H), 4.12-4.23 (tn, 4H), 5.07 (d, J=6 Hz, 1H), 8.08 (br t, J=6 Hz, 2H), 8.16 (br t, J=6 Hz, 1H), 8.77 (d, J=9 Hz, 1H).

### (e) Preparation of BHALys[Lys]₃₂[α-Succ-Gly-Gly-Gly-amino diethylmalonate]₃₂[ε-PEG₂₂₀₀]₃₂

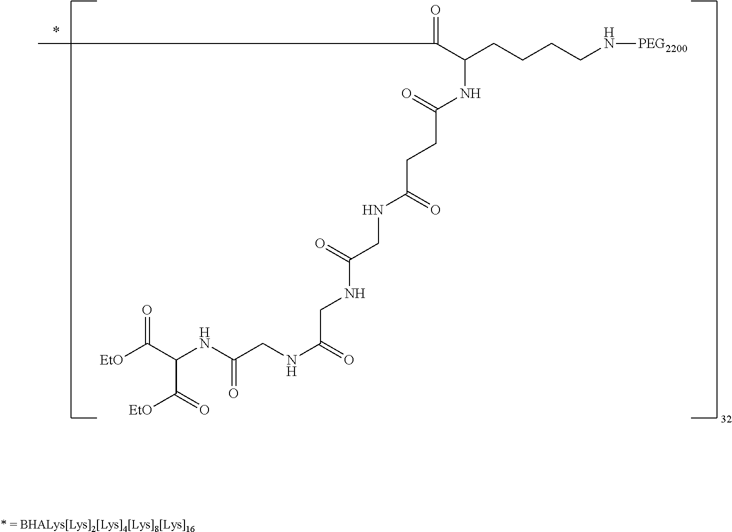

Prepared using Procedure C, step 1 above, using succinyl-triglycine amino diethylmalonate (140 mg, 0.31 mmol) and BHALys[Lys]₃₂[α-NH₂.TFA]₃₂[ε-PEG₂₂₀₀]₃₂ (500 mg, 0.007 mmol). Desired product was obtained after ultrafiltration through a 10 kDa Omega Centramate™ membrane. The retentate was freeze dried to give 550 mg (92%) of the desired product as a white solid. HPLC: Rf (min)=8.65 min. ¹H-nmr (300 MHz, CD₃OD) δ (ppm): 1.30 (t, J=6 Hz, 187H), 1.32-1.96 (m, 382H), 2.55-2.77 (tn, 108H), 3.21-3.28 (m, 123H), 3.37 (s, 89H), 3.40-3.90 (m, 5500H), 3.90-4.01 (m, 184H), 4.21-4.29 (m, 172H), 5.19 (s, 14H), 7.31 (br s, 14H). Theoretical molecular weight of conjugate with 27 sites pegylated and 32 linkers: 83.4 kDa. ¹H NMR suggests 27 linkers/dendrimer. Actual molecular weight is approximately 81.2 kDa.

### (f) Preparation of BHALys[Lys]₃₂[α-Succ-Gly-Gly-Gly-amino malonic acid]₃₂[ε-PEG₂₂₀₀]₃₂

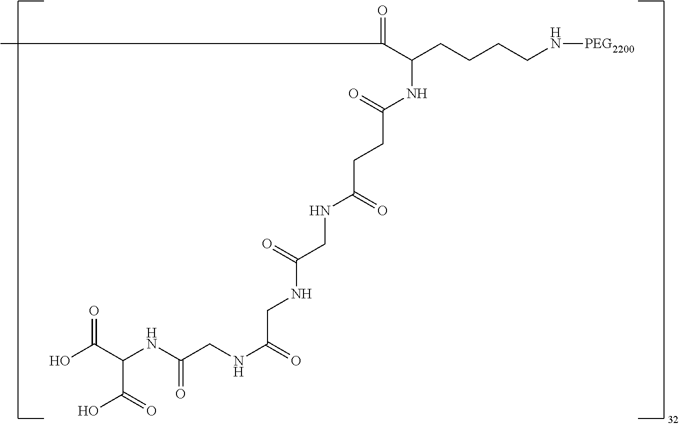

Prepared using Procedure C, step 2 above, using BHALys[Lys]₃₂[α-succinyl-triglycine amino diethylmalonate]₃₂[ε-PEG₂₂₀₀]₃₂ (520 mg, 0.006 mmol). Desired product was obtained after ultrafiltration through a 10 kDa Omega Centramate™ membrane. The retentate was freeze dried to give 470 mg (92%) of the desired product as a white solid. HPLC: Rf (min)=8.55 min. ¹H-nmr (300 MHz, CD₃OD) δ (ppm): 1.25-2.02 (m, 341H), 2.47-2.82 (m, 126H), 3.07-3.31 (m, 131H), 3.38 (s, 93H), 3.40-3.98 (n, 5498H), 3.98-4.32 (m, 235H), 7.31 (br s, 9H). ¹H NMR suggests 27 linkers/dendrimer. Actual molecular weight is approximately 79.7 kDa.

### (g) Preparation of BHALys[Lys]₃₂[α-Succ-Gly-Gly-Gly-amino malonic acid-N,O—Pt-1R,2R-(DACH)]₃₂[ε-PEG₂₂₀₀]₃₂

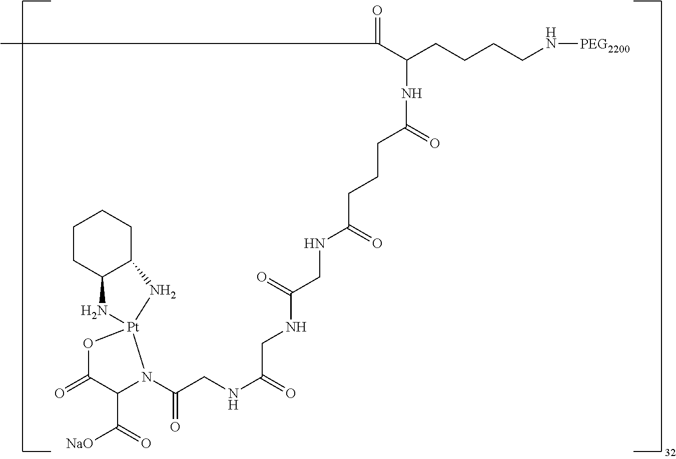

Prepared using Procedure D above, using BHALys[Lys]₃₂[α-succinyl-triglycine amino malonic acid]₃₂[ε-PEG₂₂₀₀]₃₂ (446 mg, 0.006 mmol). Desired product was obtained after freeze drying the retentate from the ultrafiltration to yield 430 mg of platinum coordinated product as a brown solid. HPLC: Rf (min)=8.39 min. ¹H-nmr (300 MHz, CD₃OD) δ (ppm): 0.79-1.92 (m, 554H), 1.92-2.14 (m, 206H), 3.38 (s, 99H), 3.40-3.90 (m, 5515H), 3.98-4.69 (m, 262H), 7.10-7.43 (m, 8H). ICP-OES: Determined Pt % 8.3%. This indicates there are 39 Pt containing moieties on the macromolecule, being on average 1.4 platinum atoms (in platinum containing moiety) on each of the estimated 27 linkers. There are platinum-containing moieties on about 42%, of the surface amino groups. Actual molecular weight is approximately 91.7 kDa.

### Example 2

### Preparation of BHALys[Lys]₃₂[α-Succ-Gly-Gly-Gly-amino malonic acid-O,O′—Pt-diammine]₃₂[ε-PEG₂₂₀₀]₃₂ (Compound 2)

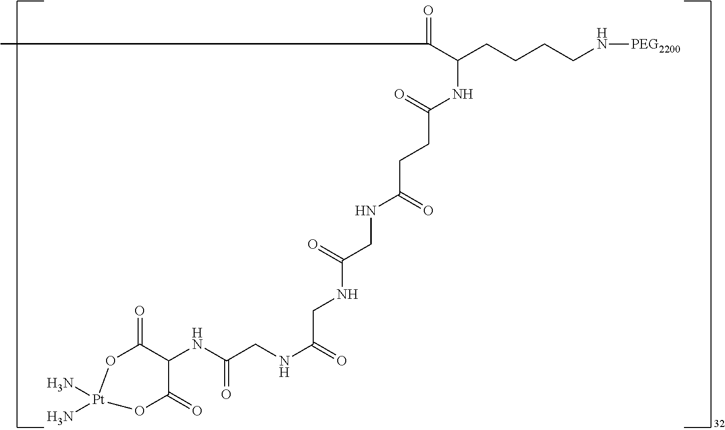

Prepared using Procedure D above, using BHALys[Lys]₃₂[α-succinyl-triglycine amino malonic acid]₃₂[Σ-PEG₂₂₀₀]₃₂ (372 mg, 0.005 mmol) and cis-diamminediaquaPlatinum(II) dinitrate solution in water (5.4 mM, 40 mL). Desired product was obtained after freeze drying half the retentate from the first ultrafiltration to yield 200 mg of O,O′-platinum coordinated product as a light brown solid. HPLC: Rf (min)=8.39 min. ¹H-nmr (300 MHz, CD₃OD) δ (ppm): 0.97-2.13 (m, 337H), 2.34-2.88 (m, 86H), 3.00-3.32 (m, 57H), 3.37 (s, 88H), 3.40-3.90 (m, 4700H), 3.97-4.70 (m, 303H), 7.14-7.48 (m, 7H). ICP-OES: Determined Pt % 4.1%. This indicates there are 17 platinum atoms (in platinum containing moiety) on about 26.5%, of the surface amino groups. Actual molecular weight of the conjugate is determined to be 83.8 kDa.

### Example 3

### Preparation of BHALys[Lys]₃₂[α-Succ-Gly-Gly-Gly-amino malonic acid-O,N—Pt-diammine]₃₂[ε-PEG₂₂₀₀]₃₂ (Compound 3)

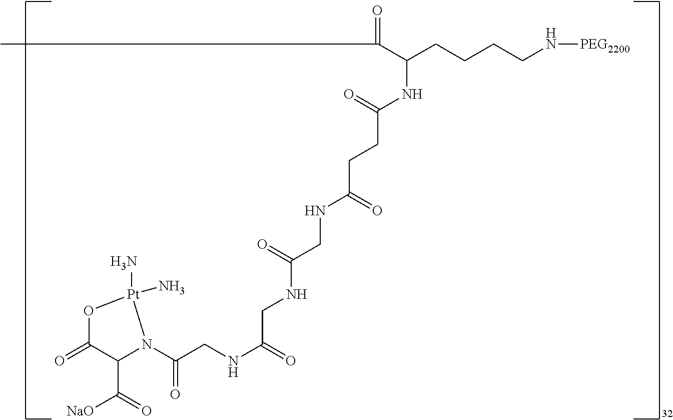

Prepared using Procedure D above, using BHALys[Lys]₃₂[α-succinyl-triglycine amino malonic acid]₃₂[ε-PEG₂₂₀₀]₃₂ (372 mg, 0.005 mmol) and cis-diamminediaquaPlatinum(II) dinitrate solution in water (5.4 mM, 40 mL). Desired product was obtained after freeze drying the retentate obtained from the second ultrafiltration to yield 170 mg of O,N′-platinum coordinated product as a light brown solid. HPLC: Rf (min)=8.39 mM. ¹H-nmr (300 MHz, CD₃OD) δ (ppm): 0.72-2.11 (m, 392H), 2.28-2.82 (m, 120H), 3.11-3.32 (m, 133H), 3.37 (s, 88H), 3.41-3.89 (m, 4700H), 3.95-4.66 (m, 233H), 7.15-7.56 (m, 8H). ICP-OES: Determined Pt % 4.0%. This indicates there are 17 platinum atoms (in platinum containing moiety) on about 26.5% of the 64 surface amino groups. Actual molecular weight of the conjugate is determined to be 83.6 kDa.

### Example 4

### Preparation of BHALys[Lys]₃₂[α-Succ-Gly-Gly-Gly-amino malonic acid-N,O—Pt-1R,2R-(DACH)]₃₂[ε-PEG₁₁₀₀]₃₂ (Compound 4)

### (a) Preparation of BHALys[Lys]₃₂[α-Succ-Gly-Gly-Gly-amino diethylmalonate]₃₂[ε-PEG₁₁₀₀]₃₂

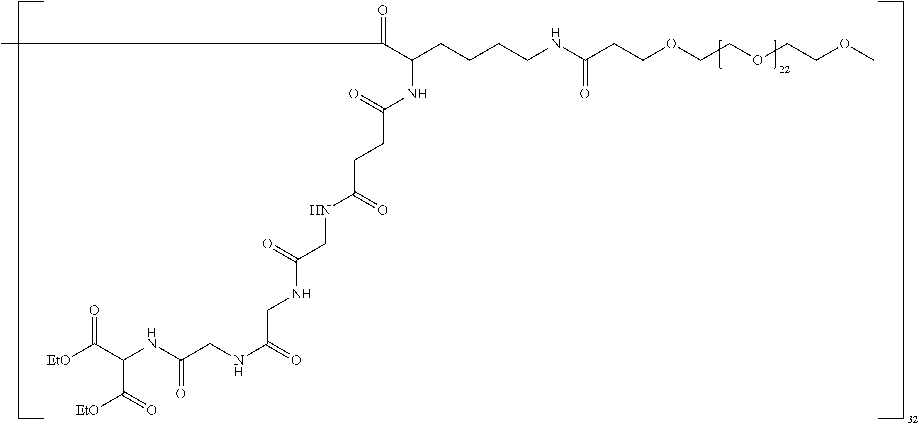

Prepared using Procedure C, step 1 above, using succinyl-triglycine amino diethylmalonate (130 mg, 0.29 mmol) and BHALys[Lys]₃₂[α-NH₂.TFA]₃₂[ε-PEG₁₁₀₀]₃₂ (366 mg, 0.009 mmol). Desired product was obtained after SEC (Sephadex™, LH20, MeOH). The appropriate fractions, as judged by HPLC, were combined and concentrated to provide the desired material 409 mg (79%). HPLC: Rf (min)=8.75 min. ¹H-nmr (300 MHz, CD₃OD) δ (ppm): 1.19 (t, J=6 Hz, 233H), 1.35-1.81 (m, 319H), 2.34-2.60 (m, 182H), 3.01-3.08 (m, 137H), 3.26 (2×s, 130H), 3.28-3.79 (m, 2800H), 3.84-3.93 (m, 118H), 4.07-4.23 (m, 165H), 5.08 (s, 20H), 7.15-7.24 (m, 11H). Theoretical molecular weight of conjugate with 32 linkers: 57.3 kDa. ¹H NMR suggests 32 linkers/dendrimer. Actual molecular weight is approximately 57.3 kDa.

### (b) Preparation of BHALys[Lys]₃₂[α-Succ-Gly-Gly-Gly-amino malonic acid]₃₂[ε-PEG₁₁₀₀]₃₂

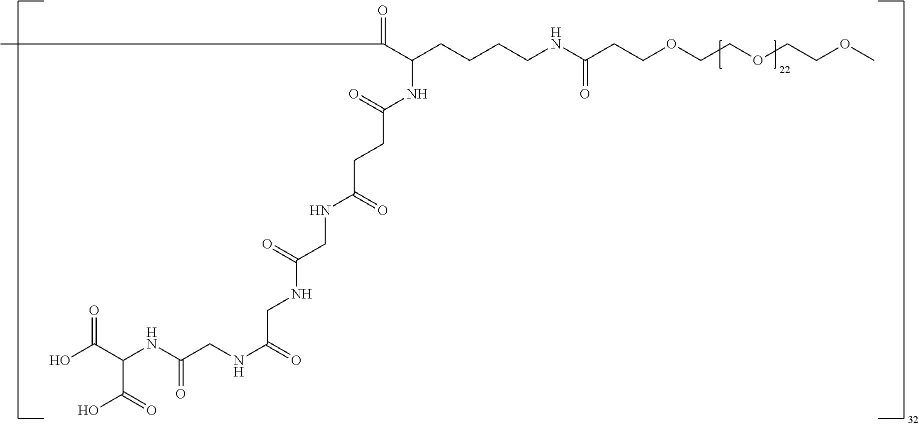

Prepared using Procedure C, step 2 above, using BHALys[Lys]₃₂[α-succinyl-triglycine amino diethylmalonate]₃₂[ε-PEG₁₁₀₀]₃₂ (405 mg, 0.007 mmol). Desired product was obtained after neutralizing the reaction mixture with CG-Amberlite™ cation exchange resin-H+ form. The reaction mixture was filtered and the filtrate was freeze dried to give 364 mg (quant.) as a white solid. HPLC: Rf (min)=8.59 min. ¹H-nmr (300 MHz, CD₃OD) δ (ppm): 1.27-1.95 (m, 319H), 2.46-2.78 (m, 155H), 3.15-3.24 (m, 76H), 3.38 (s, 91H), 3.41-3.91 (m, 2800H), 3.96-4.34 (m, 204H), 7.26-7.38 (m, 9H). ¹H NMR suggests 32 linkers/dendrimer. Actual molecular weight is approximately 55.1 kDa.

### (c) Preparation of BHALys[Lys]₃₂[α-Succ-Gly-Gly-Gly-amino malonic acid-N,O—Pt-1R,2R-(DACH)]₃₂[ε-PEG₁₁₀₀]₃₂

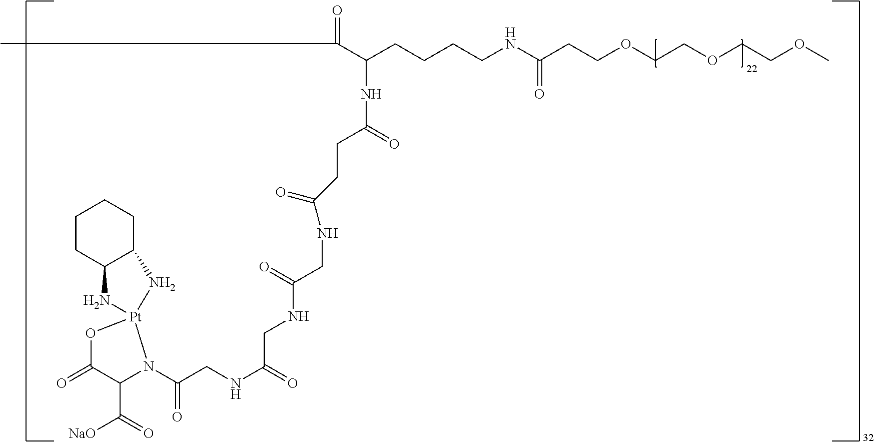

Prepared using Procedure D above, using BHALys[Lys]₃₂[α-succinyl-triglycine amino malonic acid]₃₂[ε-PEG₁₁₀₀]₃₂ (354 mg, 0.006 mmol). Desired product was obtained after freeze drying the retentate from the second ultrafiltration to yield 340 mg of platinum coordinated product as a light brown solid. HPLC: Rf (min)=8.35 min. ¹H-nmr (300 MHz, CD₃OD) δ (ppm): 0.93-2.06 (m, 454H), 2.24-2.76 (m, 246H), 3.03-3.21 (m, 105H), 3.38 (s, 109H), 3.44-3.89 (m, 2845H), 4.07-4.49 (m, 175H), 7.03-7.70 (m, 13H). ICP-OES: Determined Pt % 9.8%. This indicates there are 32 platinum atoms (in platinum containing moiety) on about 50% of the surface amino groups. Actual molecular weight of the conjugate is determined to be 65.2 kDa.

### Example 5

### Preparation of BHALys[Lys]₃₂[α-Succ-Gly-Leu-Phe-Gly-amino malonic acid-Pt-1R,2R-(DACH)]₃₂[ε-PEG₂₂₀₀]₃₂ (Compound 5)

### (a) Preparation of Boc-Gly-Leu-Phe-Gly-amino diethylmalonate

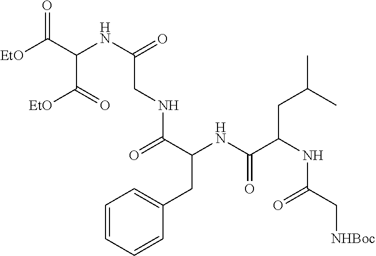 [SMILES: CCOC(=O)C(NC(=O)CNC(=O)C(Cc1ccccc1)NC(=O)C(CC(C)C)NC(=O)CNC(=O)OC(C)(C)C)C(=O)OCC]

Prepared using Procedure A, step 1 above, using Boc-Gly-Leu-Phe-Gly (4.0 g, 8.1 mmol). SiO₂ flash chromatography provided 3.5 g (66%) of product as a white solid. LCMS (C18 Column): Rf (min)=12.27. ESI (+ve) observed [M+H]⁺=650. Calculated for C₃₁H₄₇N₅O₁₀=649.33 Da. ¹H-nmr (300 MHz, CD₃OD) δ (ppm): 0.86 (d, J=6 Hz, 3H), 0.90 (d, J=6 Hz, 3H), 1.30 (t, J=6 Hz, 6H), 1.41-1.60 (m, 3H), 1.46 (s, 9H), 2.98 (dd, J=9 Hz and 15 Hz, 1H), 3.28 (dd, J=6 Hz and 15 Hz, 1H), 3.72 (s, 2H), 3.90 (d, J=15 Hz, 1H), 4.02 (d, J=15 Hz, 1H), 4.19-4.34 (m, 5H), 4.61 (dd, J=6 Hz and 9 Hz, 1H), 5.17 (s, 1H), 7.18-7.31 (m, 5H).

### (b) Preparation of H₂N-Gly-Leu-Phe-Gly-amino diethylmalonate trifluoroacetate

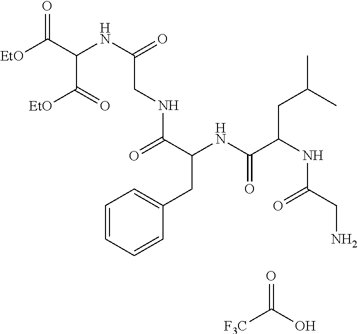 [SMILES: CCOC(=O)C(NC(=O)CNC(=O)C(Cc1ccccc1)NC(=O)C(CC(C)C)NC(=O)CN)C(=O)OCC ; O=C(O)C(F)(F)F]

Prepared using Procedure A, step 2 above, using Boc-Gly-Leu-Phe-Gly-amino diethylmalonate (3.5 g, 5.4 mmol). Desired product obtained as white solid (3.4 g, 94%). LCMS (C18 column): Rf (min)=8.50. ESI (+ve) observed [M+H]⁺=550. Calculated for C₂₆H₃₉N₅O₈=549.61 Da. ¹H-nmr (300 MHz, DMSO-d₆) δ (ppm): 0.81-0.86 (m, 6H), 1.20 (t, J=6 Hz, 6H), 1.30-1.42 (m, 2H), 1.48-1.57 (m, 1H), 2.79 (dd, J=9 Hz and 15 Hz, 1H), 3.05 (dd, J=6 Hz and 15 Hz, 1H), 3.84 (d, J=6 Hz, 2H), 4.11-4.23 (m, 4H), 4.30-4.37 (m, 1H), 4.49-4.56 (m, 1H), 5.08 (d, J=9 Hz, 1H), 7.16-7.25 (m, 5H), 7.95 (br s, 3H), 8.17-8.23 (m, 2H), 8.39 (d, J=9 Hz, 1H), 8.82 (d, J=9 Hz, 1H).

### (c) Preparation of tert-BuO-Succ-Gly-Leu-Phe-Gly-amino diethylmalonate

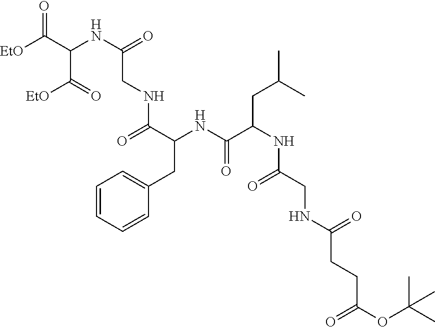 [SMILES: CCOC(=O)C(NC(=O)CNC(=O)C(Cc1ccccc1)NC(=O)C(CC(C)C)NC(=O)CNC(=O)CCC(=O)OC(C)(C)C)C(=O)OCC]

Prepared using Procedure A, step 3 above, using Gly-Leu-Phe-Gly-amino diethylmalonate trifluoroacetate (3.35 g, 5.05 mmol). Desired product obtained as off-white solid (2.3 g, 65%). LCMS (C18 column): Rf (min)=12.77. ESI (+ve) observed [M+H]⁺=706. Calculated for C₃₄H₅₁N₅O₁₁=705.35 Da. ¹H-nmr (300 MHz, DMSO-d₆) δ (ppm): 0.79 (d, J=6 Hz, 3H), 0.83 (d, J=6 Hz, 3H), 1.20 (t, J=6 Hz, 6H), 1.31-1.37 (m, 2H), 1.37 (s, 9H), 1.44-1.54 (m, 1H), 2.33-2.43 (m, 4H), 2.81 (dd, J=9 Hz and 15 Hz, 1H), 3.06 (dd, J=6 Hz and 15 Hz, 1H), 3.68 (d, J=6 Hz, 2H), 3.83 (d, J=6 Hz, 2H), 4.12-4.26 (m, 5H), 4.45-4.52 (m, 1H), 5.09 (d, J=6 Hz, 1H), 7.15-7.27 (m, 5H), 7.91 (t, J=9 Hz, 2H), 8.08-8.13 (m, 2H), 8.76 (d, J=6 Hz, 1H).

### (d) Preparation of HO-Succ-Gly-Leu-Phe-Gly-amino diethylmalonate

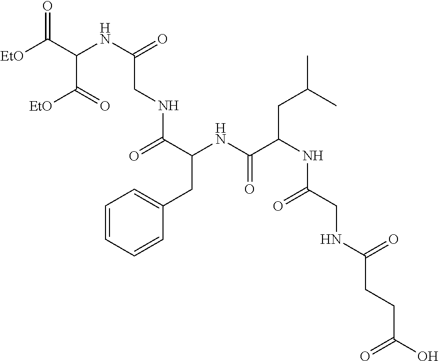 [SMILES: CCOC(=O)C(NC(=O)CNC(=O)C(Cc1ccccc1)NC(=O)C(CC(C)C)NC(=O)CNC(=O)CCC(=O)O)C(=O)OCC]

Prepared using Procedure A, step 4 above, using tert-butyl protected succinyl-Gly-Leu-Phe-Gly-amino diethylmalonate (2.3 g, 3.3 mmol). Desired product obtained as off-white solid (2.1 g, quant.). LCMS (C18 column): Rf (min)=10.50. ESI (+ve) observed [M+H]⁺=650. Calculated for C₃₀H₄₃N₅O₁₁=649.29 Da. ¹H-nmr (300 MHz, DMSO-d₆) δ (ppm): 0.79 (d, J=6 Hz, 3H), 0.83 (d, J=6 Hz, 3H), 1.20 (t, J=6 Hz, 6H), 1.32-1.40 (m, 2H), 1.45-1.56 (m, 1H), 2.36-2.45 (m, 4H), 2.81 (dd, J=9 Hz and 15 Hz, 1H), 3.05 (dd, J=6 Hz and 15 Hz, 1H), 3.68 (d, J=6 Hz, 2H), 3.83 (d, J=6 Hz, 2H), 4.13-4.26 (m, 5H), 4.45-4.53 (m, 1H), 5.09 (d, J=6 Hz, 1H), 7.17-7.27 (m, 5H), 7.87 (d, J=9 Hz, 1H), 7.93 (d, J=9 Hz, 1H), 8.09-8.14 (m, 2H), 8.76 (d, J=9 Hz, 1H).

### (e) Preparation of BHALys[Lys]₃₂[α-Succ-Gly-Leu-Phe-Gly-amino diethylmalonate]₃₂[ε-PEG₂₂₀₀]₃₂

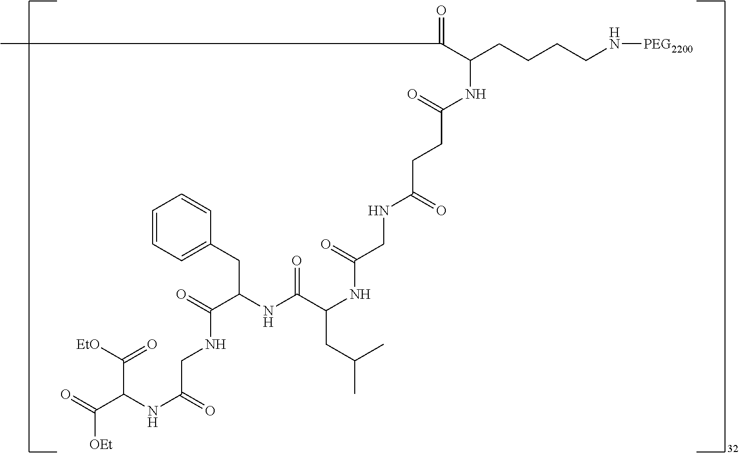

Prepared using Procedure C, step 1 above, using succinyl-Gly-Leu-Phe-Gly-amino diethylmalonate (232 mg, 0.36 mmol) and BHALys[Lys]₃₂[α-NH₂.TFA]₃₂[ε-PEG₂₂₀₀]₃₂ (400 mg, 0.006 mmol). Desired product was obtained after SEC (Sephadex™, LH20, MeOH). The appropriate fractions, as judged by HPLC, were combined and concentrated to provide the desired material 370 mg (72%). HPLC: Rf (min)=8.72 min. ¹H-nmr (300 MHz, CD₃OD) δ (ppm): 0.85-0.91 (m, 193H), 1.26-2.03 (m, 642H), 2.43-2.72 (m, 114H), 2.86-3.03 (m, 65H), 3.16-3.28 (m, 117H), 3.36 (s, 83H), 3.40-3.90 (m, 5530H), 3.96-4.06 (m, 117H), 4.19-4.63 (m, 238H), 5.20 (s, 13H), 7.18-7.26 (m, 159H). Theoretical molecular weight of conjugate with 27 sites pegylated and 32 linkers: 92.0 kDa. ¹H NMR suggests 30 linkers/dendrimer. Actual molecular weight is approximately 90.8 kDa.

### (f) Preparation of BHALys[Lys]₃₂[α-Succ-Gly-Leu-Phe-Gly-amino malonic acid]₃₂[ε-PEG₂₂₀₀]₃₂

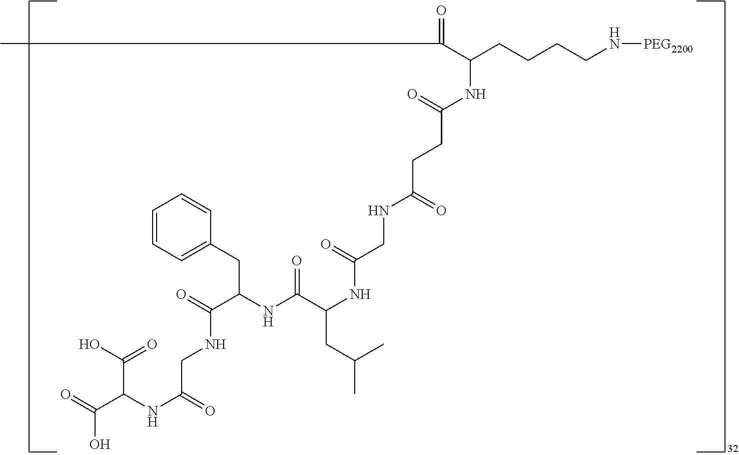

Prepared using Procedure C, step 2 above, using BHALys[Lys]₃₂[α-succinyl-Gly-Leu-Phe-Gly-amino diethylmalonate]₃₂[ε-PEG₂₂₀₀]₃₂ (370 mg, 0.004 mmol). Desired product was obtained after neutralizing the reaction mixture with CG-Amberlite™ cation exchange resin-H+ form. The reaction mixture was filtered and the filtrate was freeze dried to give 350 mg (97%) as a white solid. HPLC: Rf (min)=8.56 min. ¹H-nmr (300 MHz, CD₃OD) δ (ppm): 0.80-0.86 (m, 230H), 1.13-2.07 (m, 505H), 2.33-2.80 (m, 173H), 3.05-3.27 (m, 81H), 3.27-3.37 (m, 126H), 3.37 (s, 97H), 3.41-3.90 (m, 5430H), 3.98-4.08 (m, 67H), 4.18-4.48 (m, 97H), 4.49-4.78 (m, 80H), 7.14-7.39 (m, 168H). ¹H NMR suggests 30 linkers/dendrimer. Actual molecular weight is approximately 89.1 kDa.

### (g) Preparation of BHALys[Lys]₃₂[α-Succ-Gly-Leu-Phe-Gly-amino malonic acid-N,O—Pt-1R,2R-(DACH)]₃₂[ε-PEG₂₂₀₀]₃₂

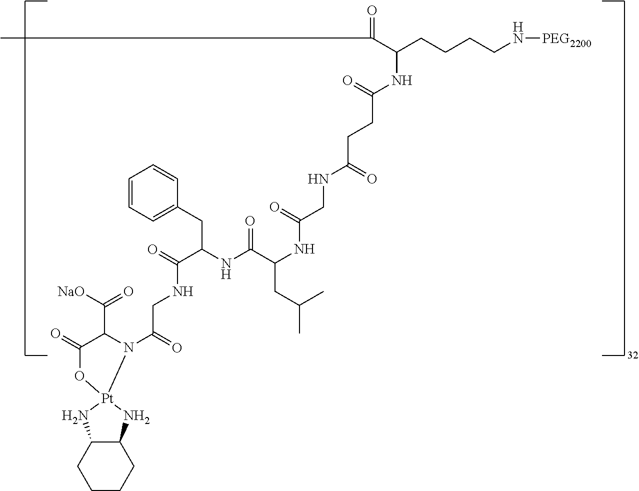

Prepared using Procedure D above, using BHALys[Lys]₃₂[α-succinyl-Gly-Leu-Phe-Gly-amino malonic acid]₃₂[ε-PEG₂₂₀₀]₃₂ (350 mg, 0.004 mmol). Desired product was obtained after freeze drying the retentate from the second ultrafiltration to yield 430 mg of N,O-chelated platinum product as a brown solid. HPLC: Rf (min)=8.49 min. ¹H-nmr (300 MHz, CD₃OD) δ (ppm): 0.28-2.14 (m, 696H), 3.37 (s, 90H), 3.43-3.88 (m, 5508H), 3.98-4.63 (m, 447H), 6.90-7.89 (m, 155H). ICP-OES: Determined Pt % 4.9%. This indicates there are 24 platinum atoms (in platinum containing moiety) on about 37.5% of the surface amino groups. Actual molecular weight is approximately 96.5 kDa.

### Example 6

### Preparation of BHALys[Lys]₃₂[α-Succ-Gly-Leu-Phe-Gly-amino malonic acid-N,O—Pt-1R,2R-(DACH)]₃₂[ε-PEG₁₁₀₀]₃₂ (Compound 6)

### (a) Preparation of BHALys[Lys]₃₂[α-Succ-Gly-Leu-Phe-Gly-amino diethylmalonate]₃₂-[ε-PEG₁₁₀₀]₃₂

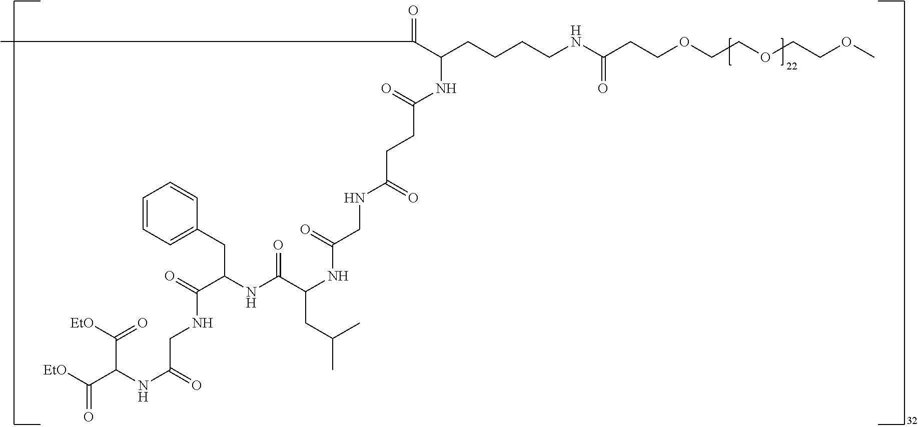

Prepared using Procedure C, step 1 above, using succinyl-Gly-Leu-Phe-Gly-amino diethylmalonate (170 mg, 0.26 mmol) and BHALys[Lys]₃₂[α-NH₂.TFA]₃₂[ε-PEG₁₁₀₀]₃₂ (259 mg, 0.006 mmol). Desired product was obtained after SEC (Sephadex™, LH20, MeOH). The appropriate fractions, as judged by HPLC, were combined and concentrated to provide the desired material 250 mg (65%). HPLC: Rf (min)=9.02 min. ¹H-nmr (300 MHz, CD₃OD) δ (ppm): 0.78-0.94 (m, 223H), 1.29 (t, J=6 Hz, 234H), 1.36-1.95 (m, 428H), 2.43-2.68 (m, 203H), 2.88-3.05 (m, 87H), 3.08-3.27 (m, 147H), 3.37 (s, 127H), 3.39-3.90 (m, 2833H), 3.97-4.07 (m, 46H), 4.23-4.32 (m, 157H), 4.55-4.76 (m, 74H), 7.11-7.39 (m, 151H). Theoretical molecular weight of conjugate with 32 linkers: 63.2 kDa. ¹H NMR suggests 28 linkers/dendrimer. Actual molecular weight is approximately 60.6 kDa.

### (b) Preparation of BHALys[Lys]₃₂[α-Succ-Gly-Leu-Phe-Gly-amino malonic acid]₃₂[ε-PEG₁₁₀₀]₃₂

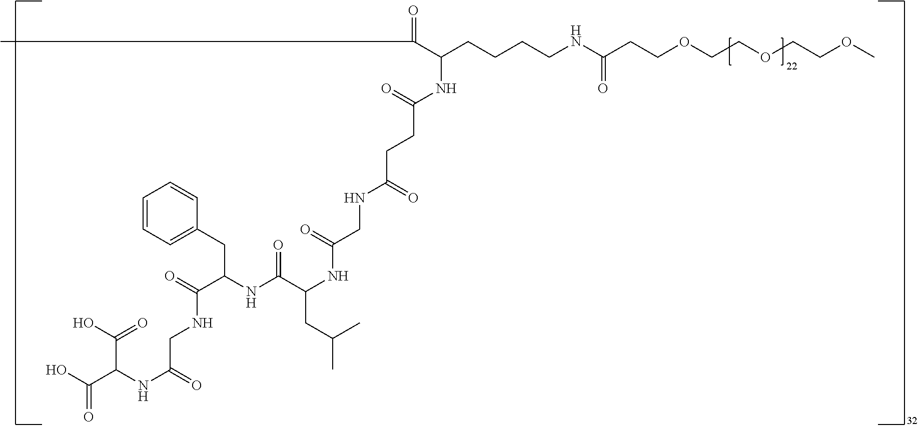

Prepared using Procedure C, step 2 above, using BHALys[Lys]₃₂[α-succinyl-Gly-Leu-Phe-Gly-amino diethylmalonate]₃₂[ε-PEG₁₁₀₀]₃₂ (240 mg, 0.004 mmol). Desired product was obtained after neutralizing the reaction mixture with CG-Amberlite™ cation exchange resin-H+ form. The reaction mixture was filtered and the filtrate was freeze dried to give 225 mg (quant.) as a white solid. HPLC: Rf (min)=8.56 min. ¹H-nmr (300 MHz, CD₃OD) δ (ppm): 0.74-0.94 (m, 172H), 1.26-1.99 (m, 477H), 2.38-2.75 (m, 181H), 2.83-3.05 (m, 75H), 3.09-3.25 (m, 117H), 3.37 (s, 94H), 3.40-3.90 (m, 2825H), 3.93-4.06 (m, 33H), 4.17-4.48 (m, 76H), 4.60-4.77 (m, 24H), 7.11-7.43 (m, 130H). ¹H NMR suggests 26 linkers/dendrimer. Actual molecular weight is approximately 58.7 kDa.

### (c) Preparation of BHALys[Lys]₃₂[α-Succ-Gly-Leu-Phe-Gly-amino malonic acid-Pt-1R,2R-(DACH)]₃₂[ε-PEG₁₁₀₀]₃₂

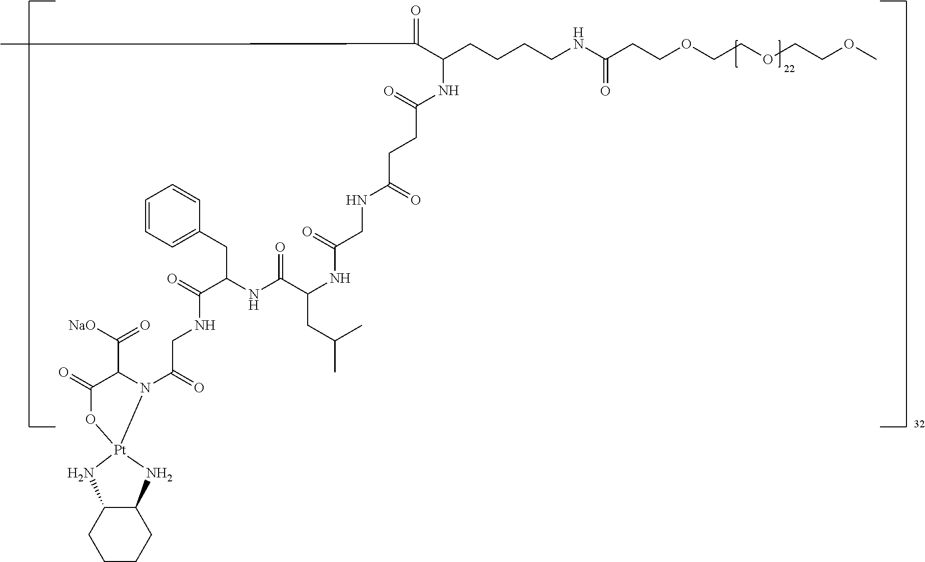

Prepared using Procedure D above, using BHALys[Lys]₃₂[α-succinyl-Gly-Leu-Phe-Gly-amino malonic acid]₃₂[ε-PEG₁₁₀₀]₃₂ (120 mg, 0.002 mmol) and cis-diaqua-1R,2R-DACH-Platinum(II) dinitrate solution in water (29.3 mM, 5 mL). Desired product was obtained after freeze drying the retentate from the second ultrafiltration to yield 74 mg of N,O-chelated platinum product as a light brown solid. HPLC: Rf (min)=8.37 min. ¹H-nmr (300 MHz, CD₃OD) δ (ppm): 0.73-1.04 (m, 140H), 1.20-2.08 (m, 540H), 2.37-2.77 (m, 301H), 3.04-3.25 (m, 85H), 3.38 (s, 123H), 3.41-3.87 (m, 2893H), 4.28-4.63 (m, 101H), 7.11-7.39 (m, 110H). ICP-OES: Determined Pt % 8.4%. This suggests there are 29 platinum atoms (in platinum containing moiety) on about 45% of the surface amino groups. Actual molecular weight is approximately 67.6 kDa.

### Example 7

### Preparation of BHALys[Lys]₃₂[α-Succ-Gly-Leu-Phe-Gly-amino malonic acid-N,O—Pt-diammine]₃₂[ε-PEG₁₁₀₀]₃₂ (Compound 7)

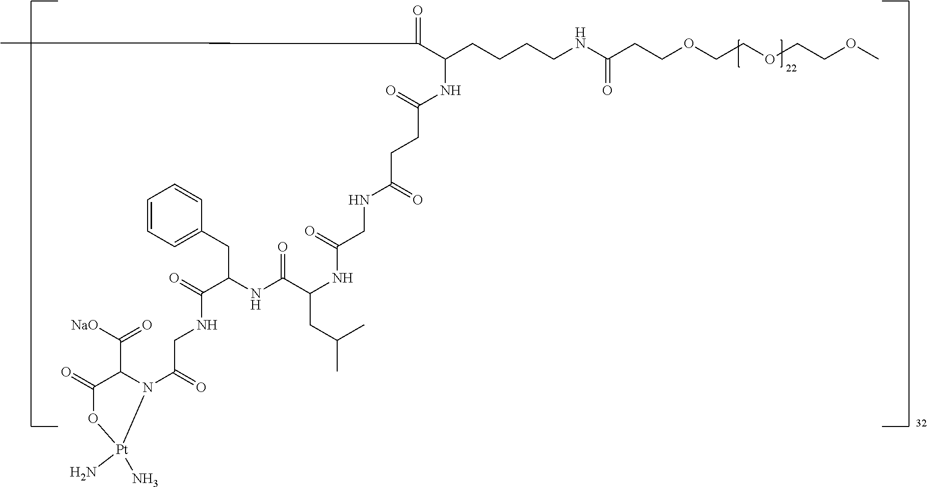

Prepared using Procedure D above, using BHALys[Lys]₃₂[α-succinyl-Gly-Leu-Phe-Gly-amino malonic acid]₃₂[ε-PEG₁₁₀₀]₃₂ (120 mg, 0.002 mmol) and cis-diamminediaquaPlatinum(II) dinitrate solution in water (3.75 mM, 25 mL). Desired product was obtained after freeze drying the retentate from the second ultrafiltration to yield 140 mg of N,O-chelated platinum product as a light brown solid. HPLC: Rf (min)=8.49 mM. ¹H-nmr (300 MHz, CD₃OD) δ (ppm): 0.74-1.03 (m, 113H), 1.19-2.06 (m, 355H), 2.28-2.77 (m, 136H), 2.79-3.02 (m, 86H), 3.26 (s, 104H), 3.28-3.89 (m, 2800H), 4.11-4.39 (m, 117H), 4.41-4.59 (m, 606H), 7.05-7.39 (m, 101H). ICP-OES: Determined Pt % 6.6%. This indicates there are 21 platinum atoms (in platinum containing moiety) on about 33% of the surface amino groups. Actual molecular weight is approximately 63.5 kDa.

### Example 8

### Preparation of BHALys[Lys]₃₂[α-Succ-Pro-Val-Gly-Leu-Ile-Gly-amino malonic acid N,O—Pt-diamine]₃₂[ε-PEG₁₁₀₀]₃₂ (Compound 8)

### (a) Preparation of Boc-Pro-Val-Gly-Leu-Ile-Gly-amino diethylmalonate

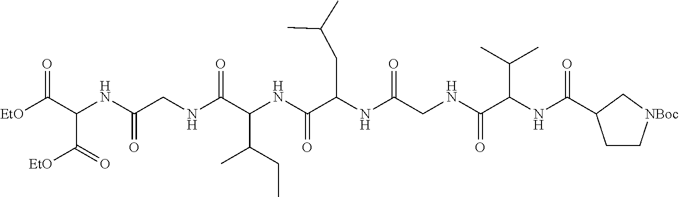 [SMILES: CCOC(=O)C(NC(=O)CNC(=O)C(NC(=O)C(CC(C)C)NC(=O)CNC(=O)C(NC(=O)C1CCN(C(=O)OC(C)(C)C)C1)C(C)C)C(C)CC)C(=O)OCC]

Prepared using Procedure A, step 1 above, using Boc-Pro-Val-Gly-Leu-Ile-Gly (500 mg, 0.76 mmol). SiO₂ flash chromatography provided 460 mg (53%) of product as an off-white solid. LCMS (C18 column): Rf (min)=13.32. ESI (+ve) observed [M+H]⁺=812. Calculated for C₃₈H₆₅N₇O₁₂=811.46 Da. ¹H-nmr (300 MHz, CD₃OD) δ (ppm): 0.89-1.03 (m, 19H), 1.17-1.24 (In, 2H), 1.29 (t, J=6 Hz, 6H,), 1.43 and 1.48 (2×s, 9H), 1.57-1.71 (m, 4H), 1.85-1.99 (m, 4H), 2.05-2.30 (m, 2H), 3.39-3.54 (m, 2H), 3.89 (s, 2H), 3.98 (d, J=3 Hz, 2H), 4.10-4.12 (m, 2H), 4.20-4.33 (m, 4H), 4.42-4.46 (m, 1H).

### (b) Preparation of NH-Pro-Val-Gly-Leu-Ile-Gly-amino diethylmalonate trifluoroacetate

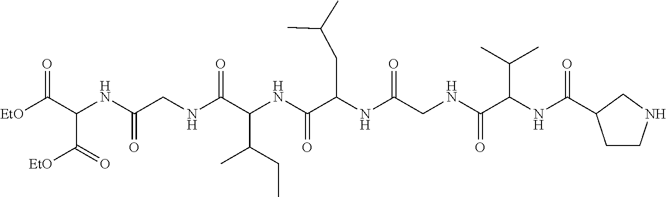 [SMILES: CCOC(=O)C(NC(=O)CNC(=O)C(NC(=O)C(CC(C)C)NC(=O)CNC(=O)C(NC(=O)C1CCNC1)C(C)C)C(C)CC)C(=O)OCC]

Prepared using Procedure A, step 2 above, using Boc-Pro-Val-Gly-Leu-Ile-Gly-amino diethylmalonate (460 mg, 0.57 mmol). Desired product obtained as white solid (468 mg, quant) LCMS (C18 column): Rf (min)=8.50. ESI (+ve) observed [M+H]⁺=712. Calculated for C₃₃H₅₇N₇O₁₀=711.41 Da. ¹H-nmr (300 MHz, DMSO-d₆) δ (ppm): 0.84-0.97 (m, 18H), 1.05-1.17 (m, 1H), 1.26 (t, J=6 Hz, 6H), 1.42-1.54 (m, 3H), 1.57-1.82 (m, 2H), 1.84-1.97 (m, 3H), 2.01-2.12 (m, 1H), 2.30-2.40 (m, 1H), 3.20-3.34 (m, 2H), 3.70-3.97 (m, 4H), 4.14-4.48 (m, 9H), 5.14 (d, J=6 Hz, 1H), 7.89 (d, J=9 Hz, 1H), 7.99 (d, J=6 Hz, 1H), 8.22 (t, J=6 Hz, 1H), 8.33 (t, J=6 Hz, 1H), 8.61 (d, J=12 Hz, 2H), 8.79 (d, J=9 Hz, 1H), 9.20 (br s, 1H).

### (c) Preparation of tert-BuO-Succ-Pro-Val-Gly-Leu-Ile-Gly-amino diethylmalonate

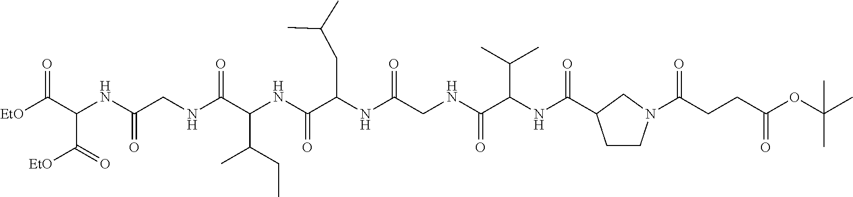 [SMILES: CCOC(=O)C(NC(=O)CNC(=O)C(NC(=O)C(CC(C)C)NC(=O)CNC(=O)C(NC(=O)C1CCN(C(=O)CCC(=O)OC(C)(C)C)C1)C(C)C)C(C)CC)C(=O)OCC]

Prepared using Procedure A, step 3 above, using Pro-Val-Gly-Leu-Ile-Gly-amino diethylmalonate trifluoroacetate (468 mg, 0.57 mmol). Desired product obtained as off-white solid (300 mg, 61%). LCMS (C18 column): Rf (min)=13.13. ESI (+ve) observed [M]⁺=868. Calculated for C₄₁H₆₉N₇O₁₃=868.03 Da. ¹H-nmr (300 MHz, DMSO-d₆) δ (ppm): 0.84-0.93 (m, 18H), 1.05-1.17 (m, 1H), 1.26 (t, J=6 Hz, 6H), 1.43 and 1.44 (2×s, 9H), 1.47-1.57 (m, 3H), 1.59-1.85 (m, 3H), 1.92-2.08 (m, 4H), 2.14-2.51 (m, 4H), 2.78-2.86 (m, 0.5H), 3.18-3.27 (m, 0.5H), 3.43-3.63 (m, 2H), 3.65-3.96 (m, 4H), 4.05-4.31 (m, 6H), 4.39-4.62 (m, 2H), 5.14 (d, J=9 Hz, 1H), 7.80-7.96 (m, 3H), 8.12-8.26 (m, 2H), 8.77 (d, J=6 Hz, 1H).

### (d) Preparation of HO-Succ-Pro-Val-Gly-Leu-Ile-Gly-amino diethylmalonate

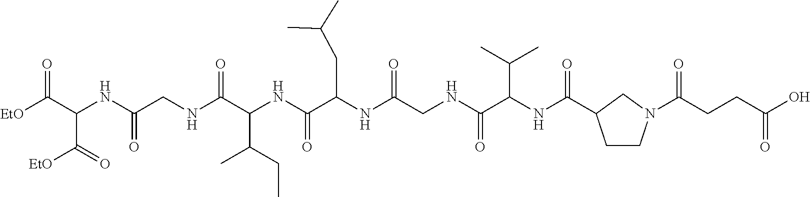 [SMILES: CCOC(=O)C(NC(=O)CNC(=O)C(NC(=O)C(CC(C)C)NC(=O)CNC(=O)C(NC(=O)C1CCN(C(=O)CCC(=O)O)C1)C(C)C)C(C)CC)C(=O)OCC]

Prepared using Procedure A, step 4 above, using bocylated succinyl-Pro-Val-Gly-Leu-Ile-Gly-amino diethylmalonate (300 mg, 0.35 mmol). Desired product obtained as off-white solid (265 mg, 94%). LCMS (C18 column): Rf (min)=10.50. ESI (+ve) observed [M+H]⁺=812. Calculated for C₃₇H₆₁N₇O₁₃=811.43 Da. ¹H-nmr (300 MHz, DMSO-d₆) δ (ppm): 1.20-1.29 (m, 18H), 1.41-1.51 (m, 1H), 1.62 (t, J=6 Hz, 6H), 1.83-1.90 (m, 3H), 1.95-2.46 (m, 9H), 2.51-2.85 (m, 4H), 3.88-4.32 (m, 6H), 4.47-4.65 (m, 4H), 4.75-4.98 (m, 2H), 5.49 (d, J=9 Hz, 1H), 8.17-8.36 (m, 3H), 8.47-8.61 (m, 2H), 9.13 (d, J=6 Hz, 1H), 12.41 (br s, 1H).

### (e) Preparation of BHALys[Lys]₃₂[α-Succ-Pro-Val-Gly-Leu-Ile-Gly-amino diethylmalonate]₃₂[ε-PEG₁₁₀₀]₃₂

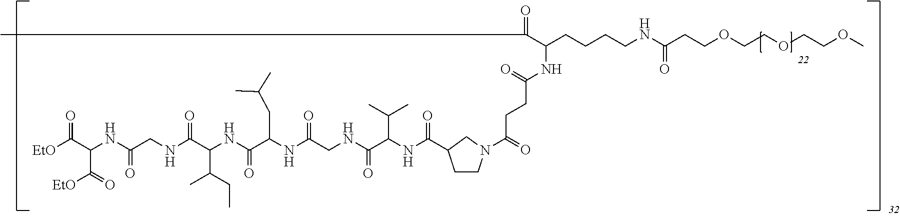

Prepared using Procedure C, step 1 above, using succinyl-Pro-Val-Gly-Leu-Ile-Gly-amino diethylmalonate (265 mg, 0.33 mmol) and BHALys[Lys]₃₂[α-NH₂.TFA]₃₂[ε-PEG₁₁₀₀]₃₂ (323 mg, 0.008 mmol). Desired product was obtained after SEC (Sephadex™, LH20, MeOH). The appropriate fractions, as judged by HPLC, were combined and concentrated to provide the desired material 410 mg (89%). HPLC: Rf (min)=9.23 min. ¹H-nmr (300 MHz, CD₃OD) δ (ppm): 0.91-0.99 (m, 449H), 1.27-2.18 (m, 712H), 2.41-2.54 (m, 114H), 3.11-3.27 (m, 105H), 3.38 (s, 101H), 3.54-3.79 (m, 2800H), 3.87-3.98 (m, 101H), 4.20-4.31 (m, 205H), 4.60 (br s, 113H), 7.31 (br s, 4H). Theoretical molecular weight of conjugate: 68.4 kDa. ¹H NMR suggests 25 linkers/dendrimer. Actual molecular weight is approximately 62.8 kDa.

### (f) Preparation of BHALys[Lys]₃₂[α-Succ-Pro-Val-Gly-Leu-Ile-Gly-amino malonic acid]₃₂[ε-PEG₁₁₀₀]₃₂

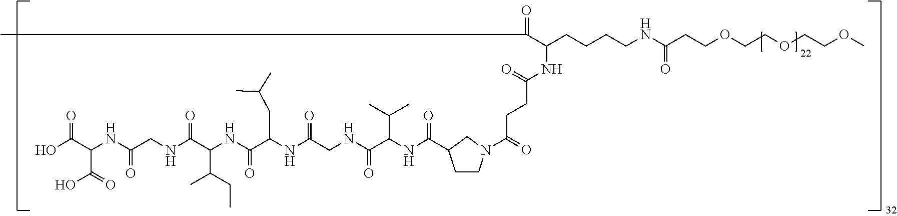

Prepared using Procedure C, step 2 above, using BHALys[Lys]₃₂[α-succinyl-Pro-Val-Gly-Leu-Ile-Gly-amino diethylmalonate]₃₂[ε-PEG₁₁₀₀]₃₂ (396 mg, 0.006 mmol). Desired product was obtained after neutralizing the reaction mixture with CG-Amberlite™ cation exchange resin-H+ form. The reaction mixture was filtered and the filtrate was freeze dried to give 385 mg (quant.) as a white solid. HPLC: Rf (min)=8.41 mM. ¹H-nmr (300 MHz, CD₃OD) δ (ppm): 0.92-0.98 (m, 558H), 1.16-2.48 (m, 807H), 2.56-2.83 (m, 101H), 3.09-3.25 (m, 113H), 3.38 (s, 95H), 3.41-3.91 (m, 2800H), 3.98-4.57 (m, 309H), 7.28 (br s, 10H). ¹H NMR suggests 25 linkers/dendrimer. Actual molecular weight is approximately 62.0 kDa.

### (g) Preparation of BHALys[Lys]₃₂[α-Succ-Pro-Val-Gly-Leu-Ile-Gly-amino malonic acid-N.O—Pt-diammine]₃₂[ε-PEG₁₁₀₀]₃₂

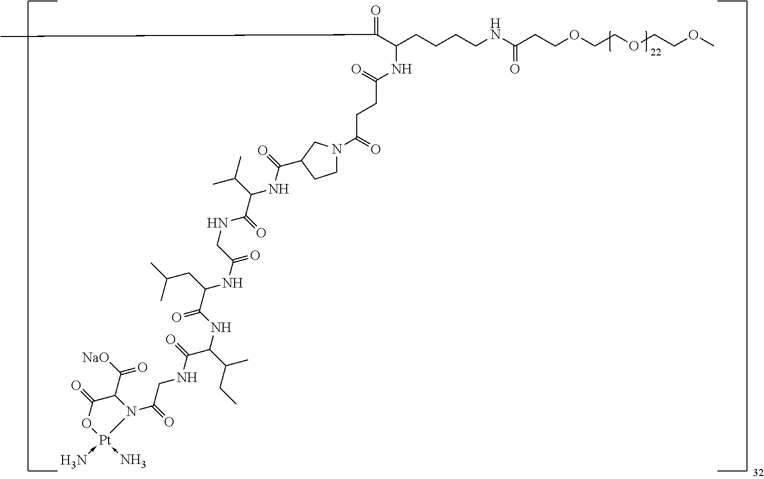

Prepared using Procedure D above, using BHALys[Lys]₃₂[α-succinyl-Pro-Val-Gly-Leu-Ile-Gly-amino malonic acid]₃₂[ε-PEG₁₁₀₀]₃₂ (385 mg, 0.006 mmol) and cis-diamminediaquaPlatinum(II) dinitrate solution (5.5 mM, 40 mL). Desired product was obtained after freeze drying the retentate from the second ultrafiltration to yield 314 mg of N,O-chelated platinum product as a brown solid. HPLC: Rf (min)=8.40 mM. ¹H-nmr (300 MHz, CD₃OD) δ (ppm): 0.70-1.11 (m, 421H), 1.28-2.23 (m, 608H), 2.39-2.94 (m, 176H), 3.10-3.31 (m, 236H), 3.38 (s, 93H), 3.42-3.89 (m, 2808H), 4.06-4.43 (m, 176H), 4.48-4.68 (m, 110H), 7.12-7.50 (m, 10H). ICP-OES: Determined Pt % 6.3%. This indicates there are 21 on about 33% platinum atoms (in platinum containing moiety) of the surface amino groups. Actual molecular weight is approximately 66.8 kDa.

### Example 9

### Preparation of BHALys[Lys]₃₂[α-2-(4-methyl phenyl (diglycolic acid)) malonic acid-Pt-1R,2R-(DACH)]₃₂[ε-PEG₂₂₀₀]₃₂ (Compound 9)

### (a) Preparation of Di-tert-butyl 2-(4-hydroxymethylphenyl) malonate

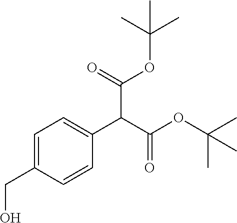 [SMILES: CC(C)(C)OC(=O)C(C(=O)OC(C)(C)C)c1ccc(CO)cc1]

Prepared using Procedure B, step 1 above, using di-tert-butyl 2-(4-formylphenyl) malonate (1.0 g, 3.12 mmol). Desired product 1.0 g (quant.) was obtained as a white solid. LCMS (C18 column): Rf (min)=13.53. ESI (+ve) observed [M+Na]⁺=345. Calculated for C₁₈H₂₆O₅=322.39 Da. ¹H-nmr (300 MHz, CD₃OD) δ (ppm): 1.39 (s, 18H), 4.36 (s, 1H), 4.62 (m, 2H), 7.27-7.33 (m, 4H).

### (b) Preparation of Di-tert-butyl 2-(4-methyl phenyl (diglycolic acid)) malonate

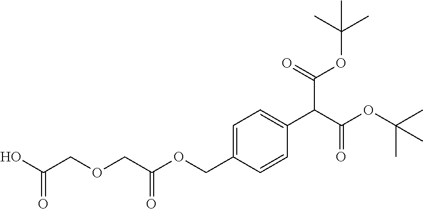 [SMILES: CC(C)(C)OC(=O)C(C(=O)OC(C)(C)C)c1ccc(COC(=O)COCC(=O)O)cc1]

Prepared using Procedure B, step 2 above, using di-tert-butyl 2-(4-hydroxymethylphenyl) malonate (50 mg, 0.16 mmol). Desired product obtained as white solid (86 mg, quant.). LCMS (C18 column): Rf (min)=5.49. ESI (+ve) observed [M+Na]⁺=461. Calculated for C₂₂H₃₀O₉=438.46 Da. ¹H-nmr (300 MHz, CD₃OD) δ (ppm): 1.17 (t, J=6 Hz, 3H), 1.35 (s, 18H), 3.06 (q, J=6 Hz, 4H), 3.99 (s, 2H), 4.16 (s, 2H), 4.42 (s, 1H), 5.08 (s, 2H), 7.24-7.27 (m, 4H).

### (c) Preparation of BHALys[Lys]₃₂[α-di-tert-butyl 2-(4-methyl phenyl (diglycolic acid)) malonate]₃₂[ε-PEG₂₂₀₀]₃₂

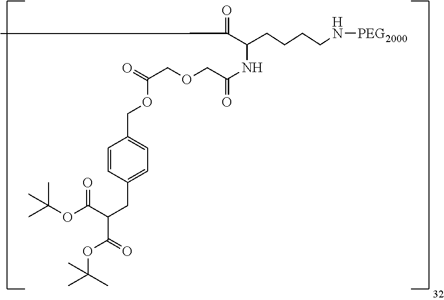

Prepared using Procedure C, step 1 above, using di-tert-butyl 2-(4-methyl phenyl (diglycolic acid) malonate (86 mg, 0.16 mmol) and BHALys[Lys]₃₂[α-NH₂.TFA]₃₂[ε-PEG₂₂₀₀]₃₂ (240 mg, 0.004 mmol) and N-methyl morpholine as the base. Desired product was obtained after SEC (Sephadex™, LH20, MeOH). The appropriate fractions, as judged by HPLC, were combined and concentrated to provide the desired material 192 mg (66%). HPLC: Rf (min)=9.05 min. ¹H-nmr (300 MHz, CD₃OD) δ (ppm): 1.30-1.93 (m, 752H), 3.19-3.28 (m, 106H), 3.37 (s, 88H), 3.40-3.90 (m, 5258H), 3.98-4.57 (m, 244H), 5.23 (br s, 36H), 7.26-7.41 (m, 88H). Theoretical molecular weight of conjugate: 82.1 kDa. ¹H NMR suggests 20 linkers/dendrimer. Actual molecular weight is approximately 75.8 kDa.

### (d) Preparation of BHALys[Lys]₃₂[α-2-(4-methyl phenyl (diglycolic acid)) malonic acid]₃₂[ε-PEG₂₂₀₀]₃₂

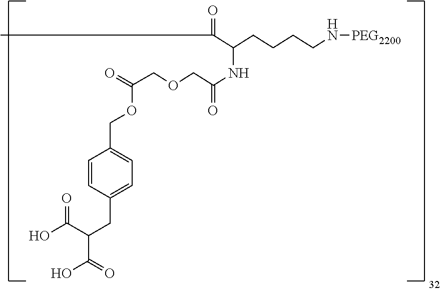

To a solution of BHALys[Lys]₃₂[α-di-tert-butyl 2-(4-methyl phenyl (diglycolic acid)) malonate]₃₂[ε-PEG₂₂₀₀]₃₂ (190 mg, 0.003 mmol) in dichloromethane (3 mL) was added trifluoroacetic acid (1.18 mL, 15.5 mmol). The reaction mixture was left to stir for 15 hours at room temperature. Volatiles were evaporated over rotary evaporator and the residue obtained was dissolved in deionised water and freeze dried to give 148 mg (80%) of the desired product as a white solid. HPLC: Rf (min)=8.44 min. ¹H NMR (CD₃OD, 300 MHz): 1.16-1.92 (m, 401H), 3.13-3.24 (m, 113H), 3.37 (s, 84H), 3.39-3.89 (m, 5250H), 3.99-4.39 (m, 212H), 5.22 (br s, 38H), 7.29-7.49 (m, 81H). ¹H NMR suggests 20 linkers/dendrimer. Actual molecular weight is approximately 73.6 kDa.

### (e) Preparation of BHALys[Lys]₃₂[α-2-(4-methyl phenyl (diglycolic acid)) malonic acid-Pt-1R,2R-(DACH)]₃₂[ε-PEG₂₂₀₀]₃₂

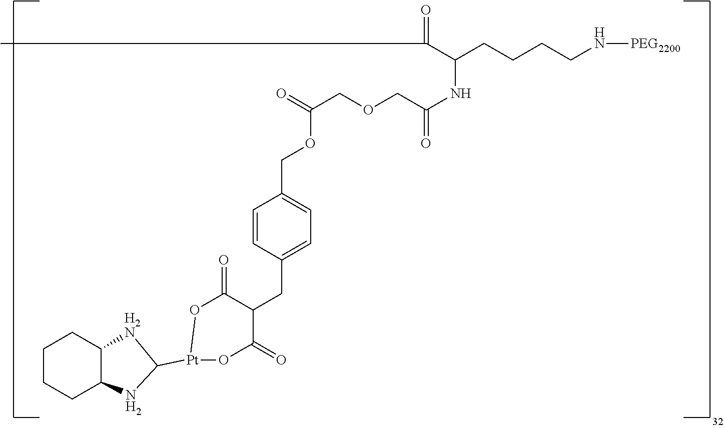

Prepared using Procedure D above, using BHALys[Lys]₃₂[α-2-(4-methyl phenyl (diglycolic acid)) malonic acid]₃₂[ε-PEG₂₂₀₀]₃₂ (285 mg, 0.004 mmol) and cis-diaqua-1R,2R-DACH-Platinum(II) dinitrate solution (9.1 mM, 13 mL). Desired product was obtained after freeze drying the retentate from the first ultrafiltration to yield 265 mg of platinum coordinated product as a brown solid. HPLC: Rf (min)=8.52 min. ¹H-nmr (300 MHz, CD₃OD) δ (ppm): 0.99-2.34 (m, 381H), 3.17-3.31 (m, 33H), 3.37 (s, 83H), 3.40-3.90 (m, 5250H), 3.96-4.08 (m, 99H), 4.25-4.51 (m, 125H), 4.55-4.63 (m, 53H), 4.99 (s, 14H), 7.04-7.62 (m, 62H). ICP-OES: Determined Pt % 4.8%. This indicates there are 20 platinum atoms (in platinum containing moiety) on about 31% of the surface amino groups. Actual molecular weight is approximately 79.7 kDa.

### Example 10

### Preparation of BHALys[Lys]₃₂[α-2-(4-methyl phenyl (diglycolic acid)) malonic acid-Pt-diamine]₃₂[ε-PEG₂₂₀₀]₃₂ (Compound 10)

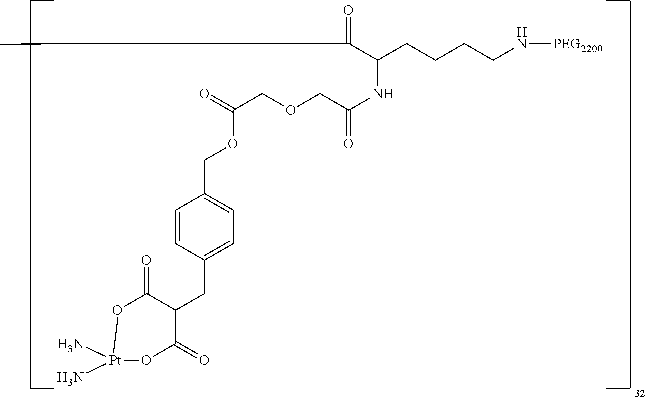

Prepared using Procedure D above, using BHALys[Lys]₃₂[α-2-(4-methyl phenyl (diglycolic acid)) malonic acid]₃₂[ε-PEG₂₂₀₀]₃₂ (148 mg, 0.002 mmol) and cis-diamminediaquo-Platinum(II) dinitrate solution (0.86 mM, 10 mL). Desired product was obtained after freeze drying the retentate from the first ultrafiltration to yield 139 mg of platinum coordinated product as a brown solid. HPLC: Rf (min)=8.67 min. ¹H-nmr (300 MHz, CD₃OD) δ (ppm): 1.02-2.01 (m, 299H), 3.03-3.17 (m, 31H), 3.26 (s, 92H), 3.28-3.79 (m, 5250H), 3.85-4.37 (m, 272H), 4.41-4.51 (m, 63H), 4.97-5.22 (m, 24H), 7.01-7.37 (m, 49H). ICP-OES: Determined Pt % 2.7%. This indicates there are 11 platinum atoms (in platinum containing moiety) on about 17% of the surface amino groups. Actual molecular weight is approximately 76.0 kDa.

### Example 11

### Preparation of BHALys[Lys]₃₂[α-2-(4-methyl phenyl (thiodiglycolic acid)) malonic acid-Pt-1R,2R-(DACH)]₃₂[ε-PEG₂₂₀₀]₃₂ (Compound 11)

### (a) Preparation of Di-tert-butyl 2-(4-methyl phenyl (thiodiglycolic acid) malonate

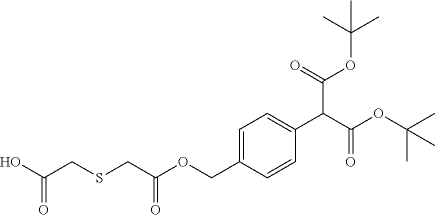 [SMILES: CC(C)(C)OC(=O)C(C(=O)OC(C)(C)C)c1ccc(COC(=O)CSCC(=O)O)cc1]

Prepared using Procedure B, step 2 above, using di-tert-butyl 2-(4-hydroxymethylphenyl) malonate (200 mg, 0.62 mmol). Desired product obtained as white solid (345 mg, quant.) as triethylamine salt. LCMS (C18 column): Rf (min)=10.56. ESI (−ve) observed [M−H]⁺=453. Calculated for C₂₂H₃₀O₈S=454.53 Da. ¹H-nmr (300 MHz, CD₃OD) δ (ppm): 1.31 (t, J=6 Hz, 3H), 1.47 (s, 18H), 3.19 (q, J=6 Hz, 2H), 3.33 (s, 2H), 3.44 (s, 2H), 4.53 (s, 1H), 5.18 (s, 2H), 7.36-7.39 (m, 4H).

### (b) Preparation of BHALys[Lys]₃₂[α-di-tert-butyl 2-(4-methyl phenyl (thiodiglycolic acid)) malonate]₃₂[ε-PEG₂₂₀₀]₃₂

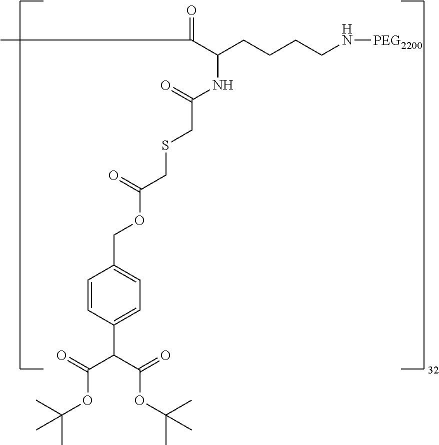

Prepared using Procedure C, step 1 above, using di-tert-butyl 2-(4-methyl phenyl (thiodiglycolic acid)) malonate (85 mg, 0.15 mmol) and BHALys[Lys]₃₂[α-NH₂.TFA]₃₂[ε-PEG₂₂₀₀]₃₂ (166 mg, 0.003 mmol) and N-methyl morpholine as the base. Desired product was obtained after SEC (Sephadex™, LH20, MeOH). The appropriate fractions, as judged by HPLC, were combined and concentrated to provide the desired material 126 mg (62%). HPLC: Rf (min)=9.17 min. ¹H-nmr (300 MHz, CD₃OD) δ (ppm): 1.24-1.81 (m, 659H), 3.05-3.18 (m, 73H), 3.26 (s, 71H), 3.28-3.78 (m, 5250H), 3.88-4.45 (m, 166H), 5.09 (br s, 48H), 7.20-7.30 (m, 83H). ¹H NMR suggests 20 linkers/dendrimer. Actual molecular weight is approximately 76.1 kDa.

### (c) Preparation of BHALys[Lys]₃₂[α-2-(4-methyl phenyl (thiodiglycolic acid)) malonic acid]₃₂[ε-PEG₂₂₀₀]₃₂

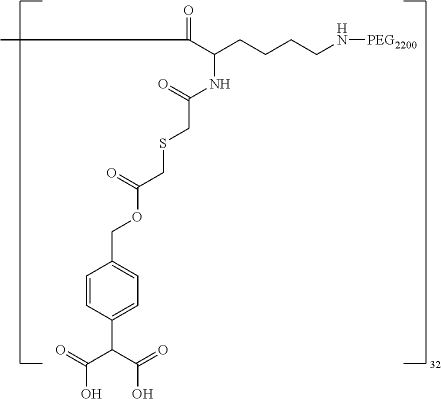

Prepared using the procedure in Example 9, Step d. Desired product obtained as a white solid (110 mg, quant.). HPLC: Rf (min)=8.44 min ¹H NMR (CD₃OD, 300 MHz): 1.09-1.88 (m, 352H), 3.05-3.20 (m, 85H), 3.26 (s, 87H), 3.28-3.79 (m, 5249H), 3.89-4.63 (m, 140H), 5.09 (br s, 38H), 7.29-7.38 (m, 70H). ¹H NMR suggests 20 linkers/dendrimer. Actual molecular weight is approximately 73.9 kDa.

### (d) Preparation of BHALys[Lys]₃₂[α-2-(4-methyl phenyl (thiodiglycolic acid)) malonic acid-Pt-1R,2R-(DACH)]₃₂[ε-PEG₂₂₀₀]₃₂

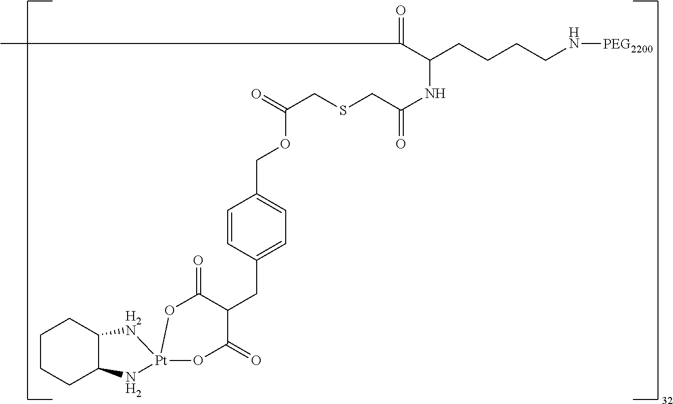

Prepared using Procedure D above, using BHALys[Lys]₃₂[α-2-(4-methyl phenyl (thiodiglycolic acid)) malonic acid]₃₂[ε-PEG₂₂₀₀]₃₂ (110 mg, 0.002 mmol) and cis-diaqua-1R,2R-DACH Platinum(II) dinitrate solution (23.6 mM, 3 mL). Desired product was obtained after freeze drying the retentate from the first ultrafiltration to yield 101 mg of platinum coordinated product as a brown solid. HPLC: Rf (min)=8.44 min. ¹H-nmr (300 MHz, CD₃OD) δ (ppm): 1.06-2.74 (m, 544H), 3.27 (s, 95H), 3.40-3.90 (m, 5250H), 4.03-4.58 (m, 168H), 7.19-7.49 (m, 44H). ICP-OES: Determined Pt % 4.9%. This indicates there are 20 platinum atoms (in platinum containing moiety) on about 33% of the surface amino groups. Actual molecular weight is approximately 80.1 kDa

### Example 12

### Preparation of BHALys[Lys]₃₂[α-thiodiglycolic acid-4-oxy-1,1-cyclohexane dicarboxylic acid —Pt-1R,2R-(DACH)]₃₂[ε-PEG₂₂₀₀]₃₂ (Compound 12)

### (a) Preparation of 4-hydroxy-1,1-cyclohexane dicarboxylic acid-1,1-bis (tert-butyl) ester

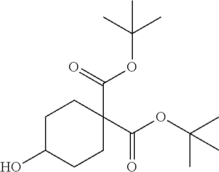 [SMILES: CC(C)(C)OC(=O)C1(C(=O)OC(C)(C)C)CCC(O)CC1]

To a solution of 4-oxo-1,1-cyclohexanedicarboxylic acid 1,1-bis (tert-butyl) ester (504 mg, 1.69 mmol) in ethyl alcohol (20 mL) at 0° C. was added sodium borohydride (64 mg, 1.69 mmol) under nitrogen atmosphere and the mixture was stirred for 1 h and then at 25° C. overnight. Worked up as in procedure B, step 1 to obtain white solid (490 mg, 97%). LCMS (C18 column) Rf (min)=12.67. ESI (+ve) observed [M+Na]⁺=323. Calculated for C₁₆H₂₈O₅=300.39. ¹H-nmr (300 MHz, CD₃OD) δ (ppm): 1.40-1.58 (m, 2H), 1.45 (s, 9H), 1.49 (s, 9H), 1.66-1.72 (m, 2H), 1.74-1.86 (m, 2H), 2.18-2.25 (m, 2H), 3.57-3.66 (m, 1H).

### (b) Preparation of thiodiglycolic acid-4-oxy-1,1-cyclohexane dicarboxylic acid-1,1-bis (tert-butyl) ester

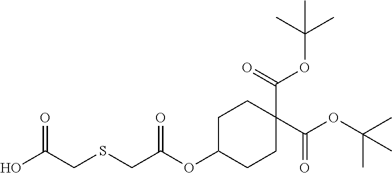 [SMILES: CC(C)(C)OC(=O)C1(C(=O)OC(C)(C)C)CCC(OC(=O)CSCC(=O)O)CC1]

Prepared using procedure B, step 2 using 4-hydroxy-1,1-cyclohexane dicarboxylic acid-1,1-bis (tert-butyl) ester (300 mg, 1.00 mmol) and thiodiglycolic anhydride (264 mg, 2.00 mmol) to obtain desired product obtained 410 mg. LCMS (C8 column): Rf (min)=6.97. ESI (+ve) observed [M+Na]⁺=455.06. Calculated for C₂₀H₃₂O₈S=432.52. ¹H-nmr (300 MHz, CD₃OD) S (ppm): 1.47 (s, 9H), 1.48 (s, 9H), 1.62-1.75 (m, 2H), 1.78-1.91 (m, 4H), 2.12-2.24 (m, 2H), 3.40 (s, 2H), 3.41 (s, 2H), 4.81-4.86 (m, 1H).

### (c) Preparation of BHALys[Lys]₃₂[α-thiodiglycolic acid-4-oxy-1,1-cyclohexane dicarboxylic acid-1,1-bis (tert-butyl) ester]₃₂[ε-PEG₂₂₀₀]₃₂

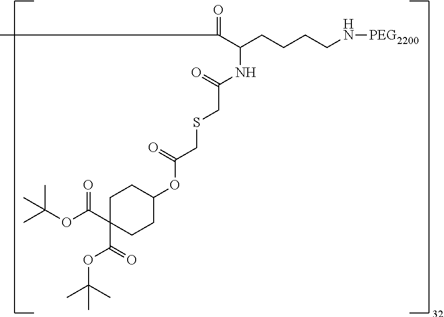

Prepared using Procedure C, step 1 above, using thiodiglycolic acid-4-oxy-1,1-cyclohexane dicarboxylic acid-1,1-bis (tert-butyl) ester (410 mg, 0.95 mmol) and BHALys[Lys]₃₂[α-NH₂.TFA]₃₂[ε-PEG₂₂₀₀]₃₂ (1.9 g, 0.02 mmol) and DIPEA as the base. Desired product was obtained after freeze drying the retentate from the ultrafiltration to provide the desired material 2.0 g (88%). HPLC: Rf (min)=9.03 min. ¹H-nmr (300 MHz, CD₃OD) δ (ppm): 1.35-2.25 (m, 1305H), 3.22-3.29 (m, 80H), 3.37 (s, 113H), 3.40-4.01 (m, 7901H), 4.19-4.54 (m, 81H), 7.22-7.40 (m, 10H). ¹H NMR suggests 32 linkers/dendrimer. Actual molecular weight is approximately 104.9 kDa.

### (d) Preparation of BHALys[Lys]₃₂[α-thiodiglycolic acid-4-oxy-1,1-cyclohexane dicarboxylic acid]₃₂[ε-PEG₂₂₀₀]₃₂

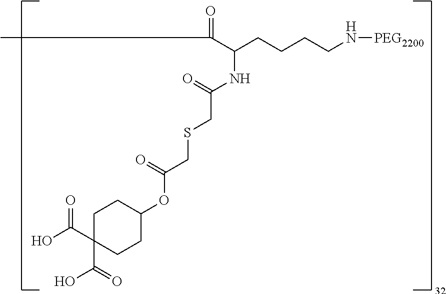

Prepared using the procedure in example 9, step D using BHALys[Lys]₃₂[α-thiodiglycolic acid-4-oxy-1,1-cyclohexane dicarboxylic acid-1,1-bis (tert-butyl) ester]₃₂[ε-PEG₂₂₀₀]₃₂ (2.0 g, 0.019 mmol) and trifluoroacetic acid (7.5 mL, 97.9 mmol). Desired product was obtained as a white solid (1.9 g, quant.). HPLC: Rf (min)=9.16 min. ¹H NMR (CD₃OD, 300 MHz) δ (ppm): 1.23-2.41 (m, 620H), 3.19-3.29 (m, 91H), 3.37 (s, 111H), 3.40-4.06 (m, 7821H), 4.20-4.72 (m, 69H), 7.22-7.47 (m, 10H). ¹H NMR suggests 30 linkers/dendrimer. Actual molecular weight is approximately 100.6 kDa.

### (e) Preparation of BHALys[Lys]₃₂[α-thiodiglycolic acid-4-oxy-1,1-cyclohexane dicarboxylic acid-Pt-1R,2R-(DACH)]₃₂[ε-PEG₂₂₀₀]₃₂

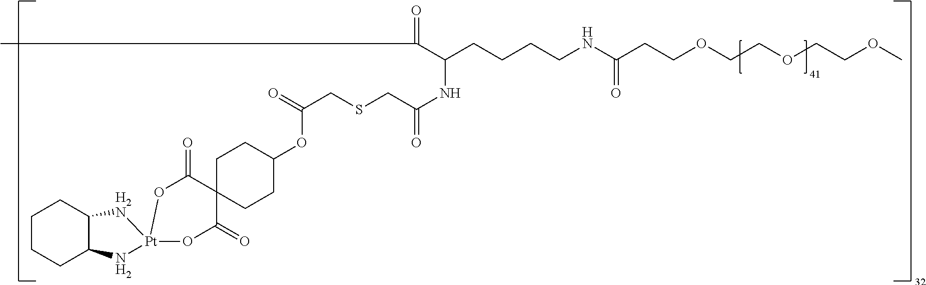

Prepared using Procedure D above, using BHALys[Lys]₃₂[α-thiodiglycolic acid-4-oxy-1,1-cyclohexane dicarboxylic acid-]₃₂[ε-PEG₂₂₀₀]₃₂ (1.96 g, 0.019 mmol) and cis-diaqua-1R,2R-DACH-Platinum(II)dinitrate solution (106 mM, 7 mL). Desired product was obtained after freeze drying the retentate from the first ultrafiltration to yield 1.8 g of platinum coordinated product as an off-white solid. HPLC: Rf (min)=7.60 min. ¹H-nmr (300 MHz, CD₃OD) δ (ppm): 0.65-2.23 (m, 562H), 2.30-2.79 (m, 51H), 3.38 (s, 113H), 3.41-3.90 (m, 7752H), 3.96-4.15 (m, 101H). ICP-OES: Determined Pt % 5.2%. This suggests there are 29 platinum atoms (in platinum containing moiety) on about 45% of the surface amino groups. Actual molecular weight is approximately 109.7 kDa.

### Example 13

### Preparation of BHALys[Lys]₃₂[α-diglycolic acid-5R-oxy-(1R,2R)-diaminocyclohexane-oxalato-Platinum (II)]₃₂[ε-PEG₂₂₀₀]₃₂ (Compound 13)

### (a) Preparation of tert-butoxy (1R,2R)-2[tert-butoxycarbonyl amino]-5R-diglycolate-cyclohexylcarbamate and tert-butoxy (1R,2R)-2[tert-butoxycarbonyl amino]-5S-diglycolate-cyclohexylcarbamate

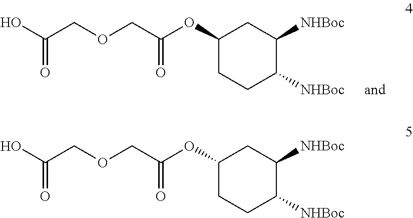 [SMILES: CC(C)(C)OC(=O)N[C@@H]1CC[C@@H](OC(=O)COCC(=O)O)C[C@H]1NC(=O)OC(C)(C)C ; CC(C)(C)OC(=O)N[C@@H]1CC[C@H](OC(=O)COCC(=O)O)C[C@H]1NC(=O)OC(C)(C)C]

To a solution of tert-butoxy (1R,2R)-2[tert-butoxycarbonyl amino]-5-hydroxy-cyclohexylcarbamate (1.29 g, 3.90 mmol) (prepared according to US Patent publication 2005/0020645) in dichloromethane (50 mL) containing activated 4 Å molecular sieves was added triethylamine (1.63 mL, 11.71 mmol) under nitrogen atmosphere and the mixture stirred at room temperature for 10 minutes. Diglycolic anhydride (906 mg, 7.81 mmol) was added and the reaction mixture was left to stir at room temperature for 15 hours. The reaction mixture was filtered and washed (3×) with phosphate buffer (5% sodium chloride, 1% sodium monophosphate and 1M aqueous hydrochloric acid in 100 mL water) at pH 3. The DCM phase was then dried with MgSO₄, filtered and concentrated. The crude was purified by preparative high performance liquid chromatography providing 5R isomer (400 mg, 23%) and 5S isomer (140 mg, 8%). Conditions: XBridge C18 column with multiple gradient 30% ACN/H₂O (1-5 min), 30-60% ACN/H₂O (5-35 min), 60% ACN/H₂O (35-47 min), 60-80% ACN/H₂O (47-50 min), 80% ACN/H₂O (50-57 min), 80-30% ACN/H₂O (57-58 min), 30% ACN/H₂O (58-70 min) with 0.1% HCOOH buffer. 5R isomer: LCMS (C18 column): R_{f} (min)=9.02. ESI (+ve) observed [M+H]⁺=447. Calculated for C₂₀H₃₄N₂O₉=446.49 Da. ¹H NMR (DMSO-d₆, 300 MHz) δ (ppm): 1.23-1.42 (m, 3H), 1.37 (s, 18H), 1.72-1.81 (m, 1H), 1.83-1.93 (m, 1H), 1.97-2.06 (m, 1H), 3.15-3.22 (m, 2H), 4.05 (s, 2H), 4.15 (s, 2H), 4.57-4.69 (m, 1H), 6.49 (d, J=9.0 Hz, 1H), 6.61 (d, J=9.0 Hz, 1H). 5S isomer: LCMS (C18 column): R_{f} (min)=8.76. ESI (+ve) observed [M+H]⁺=447. Calculated for C₂₀H₃₄N₂O₉=446.49 Da. ¹H NMR (DMSO-d₆, 300 MHz) δ (ppm): 1.36 (s, 18H), 1.48-1.65 (m, 4H), 1.72-1.79 (m, 1H), 1.83-1.92 (m, 1H), 3.43-3.53 (m, 2H), 4.11 (s, 2H), 4.19 (s, 2H), 4.59-4.67 (m, 1H), 6.54 (d, J=9.0 Hz, 1H), 6.59 (d, J=9.0 Hz, 1H).

### (b) Preparation of BHALys[Lys]₃₂[α-diglycolic acid-5R-oxy-tert-butoxy (1R,2R)-2[tert-butoxycarbonyl amino]-cyclohexylcarbamate]₃₂[ε-PEG₂₂₀₀]₃₂

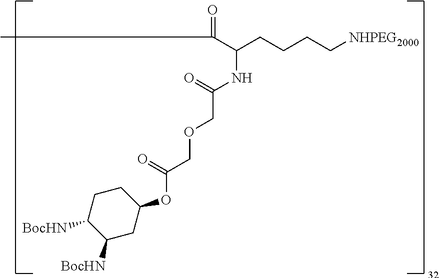

To a solution of tert-butoxy (1R,2R)-2[tert-butoxycarbonyl amino]-5R-diglycolate-cyclohexylcarbamate (120 mg, 0.27 mmol) in dimethylformamide (6 mL) was added N-methylmorpholine (40 μL, 0.36 mmol) and benzotriazol-1-yl-oxytripyrrolidinophosphonium hexafluorophosphate (PyBOP) (140 mg, 0.27 mmol). The mixture was stirred at room temperature for 10 min. before a solution of BHALys[Lys]₁₆[Lys(α-NH₂.TFA)₃₂(ε-PEG2000)₃₂] (578 mg, 0.007 mmol) and N-methylmorpholine (0.08 mL, 0.73 mmol) in dimethylformamide (4 mL) was added dropwise. The reaction mixture was stirred for 15 hours at room temperature under nitrogen atmosphere. Volatiles were evaporated over rotary evaporator and the residue taken up in water and purified via ultrafiltration (10 MWCO). The retentate was collected and freeze dried providing 630 mg (97%) of the desired product. HPLC: Rf (min)=8.89. ¹H NMR (CD₃OD, 300 MHz) δ (ppm): 1.38-2.27 (m, 1178H), 3.24-3.29 (m, 96H), 3.37 (s, 102H), 3.39-4.01 (m, 5900H), 4.12-4.44 (m, 192H) 7.26-7.33 (m, 10H). Theoretical molecular weight of conjugate: 92.7 kDa. ¹H NMR suggests 31 linkers/dendrimer.

### (c) Preparation of BHALys[Lys]₃₂[α-diglycolic acid-5R-oxy-(1R,2R)-diaminocyclohexane]₃₂[ε-PEG₂₂₀₀]₃₂

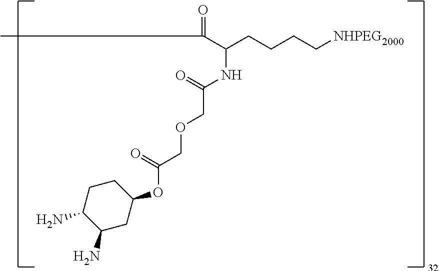

To a cooled solution of BHALys[Lys]₃₂[α-diglycolic acid-5R-oxy-tert-butoxy (1R,2R)-2[tert-butoxycarbonyl amino]-cyclohexylcarbamate]₃₂[ε-PEG₂₂₀₀]₃₂ (620 mg, 0.0067 mmol) in dichloromethane (20 mL) was added trifluoroacetic acid (5.0 mL, 65.3 mmol). The reaction mixture was left to stir for 15 hours. Volatiles were evaporated over rotary evaporator and the residue was dissolved in water and treated with Amberlite IRA-401 anion exchange resin (—OH⁻ form). When the pH was stabilised at ˜7.0, the aqueous mixture was filtered through 0.2 μM filter. The filtrate was freeze dried providing 577 mg (quant.) of the desired product as an off-white solid. HPLC: Rf (min)=7.09. ¹H NMR (CD₃OD, 300 MHz) δ (ppm): 1.39-2.60 (m, 637H), 3.25-3.28 (m, 92H), 3.37 (s, 109H), 3.40-4.01 (m, 5900H), 4.13-4.48 (m, 194H), 7.26-7.39 (m, 10H). ¹H NMR indicates 32 linkers/dendrimer (100% loading). Actual molecular weight is approximately 86.3 kDa.

### (d) Preparation of BHALys[Lys]₃₂[α-diglycolic acid-5R-oxy-(1R,2R)-diaminocyclohexane-dichloroPlatinum (II)]₃₂[ε-PEG₂₂₀₀]₃₂

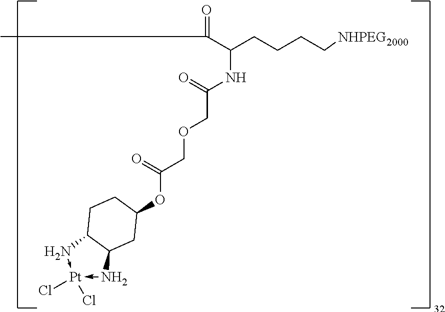

To a solution of BHALys[Lys]₃₂[α-diglycolic acid-5R-oxy-(1R,2R)-diaminocyclohexane]₃₂[ε-PEG₂₂₀₀]₃₂ (570 mg, 0.0066 mmol) in water (30 mL) was added potassium tetrachloroplatinate (96 mg, 0.23 mmol). The reaction mixture was stirred for 15 hours at room temperature whilst protected from light. The mixture was then filtered (0.22 μM) and purified via ultrafiltration (10 kDa MWCO). The retentate was freeze dried providing 626 mg (quant.) of platinum coordinated product. HPLC: Rf (min)=7.61. ¹H NMR (CD₃OD, 300 MHz) δ (ppm): 1.32-2.27 (m, 402H), 3.38 (s, 105H), 3.40-4.01 (m, 5840H), 4.20-4.55 (m, 158H), 7.19-7.47 (m, 4H). Actual molecular weight is approximately 94.8 kDa.

### (e) Preparation of BHALys[Lys]₃₂[α-diglycolic acid-5R-oxy-(1R,2R)-diaminocyclohexane-oxalato-Platinum (II)]₃₂[ε-PEG₂₂₀₀]₃₂

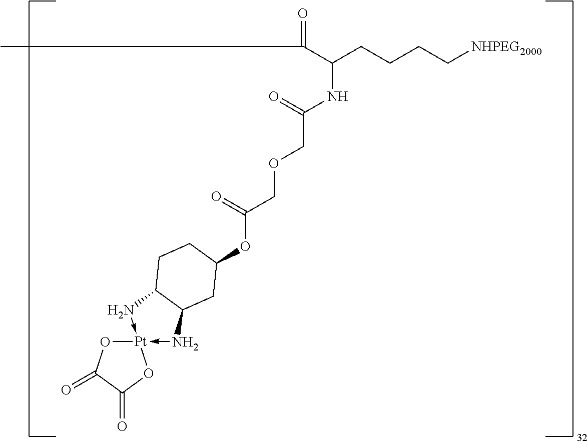

To a solution of BHALys[Lys]₃₂[α-diglycolic acid-5R-oxy-(1R,2R)-diaminocyclohexane-dichloroPlatinum (II)]₃₂[ε-PEG₂₂₀₀]₃₂) (620 mg, 0.0065 mmol) in water (20 mL) was added silver oxalate (127 mg, 0.42 mmol). The reaction mixture was stirred for 3 hours at room temperature whilst protected from light. A greyish white precipitate was filtered off through a 0.22 μm filter and then purified via ultrafiltration (10 kDa MWCO). The retentate was collected and freeze dried affording 550 mg (88%) of oxalatoplatinum (II) product. HPLC: Rf (min)=7.54. ¹H NMR (CD₃OD, 300 MHz) δ (ppm): 1.33-2.71 (m, 509H), 3.38 (s, 111H), 3.40-4.02 (n, 5900H), 4.25-4.61 (m, 159H), 7.17-7.52 (m, 11H). ICP-OES: Determined platinum content was 5.1%. This suggests there are 24 platinum atoms (in platinum containing moiety) on about 38% of the surface amino groups. Actual molecular weight is approximately 93.2 kDa.

### Example 14

### Preparation of BHALys[Lys]₃₂[α-diglycolic acid-5S-oxy-(1R,2R)-diaminocyclohexane-oxalato-Platinum (II)]₃₂[ε-PEG₂₂₀₀]₃₂ (Compound 14)

### (a) Preparation of BHALys[Lys]₃₂[α-diglycolic acid-5S-oxy-tert-butoxy (1R,2R)-2[tert-butoxycarbonyl amino]-cyclohexylcarbamate]₃₂[ε-PEG₂₂₀₀]₃₂

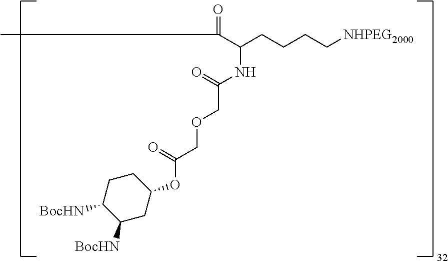

Prepared as per the procedure for synthesis of BHALys[Lys]₃₂[α-diglycolic acid-5R-oxy-tert-butoxy (1R,2R)-2[tert-butoxycarbonyl amino]-cyclohexylcarbamate]₃₂[ε-PEG₂₂₀₀]₃₂. HPLC: Rf (min)=8.73. ¹H NMR (CD₃OD, 300 MHz) δ (ppm): 1.32-2.16 (m, 1167H), 3.20-3.31 (m, 89H), 3.37 (s, 102H), 3.41-4.01 (m, 5900H), 4.12-4.48 (m, 189H), 5.22 (brs, 30H) 7.19-7.34 (m, 9H). Theoretical molecular weight of conjugate: 92.7 KDa. ¹H NMR indicates 31 linkers/dendrimer (96.9% loading).

### (b) Preparation of BHALys[Lys]₃₂[α-diglycolic acid-5S-oxy-(1R,2R) diaminocyclohexane]₃₂[ε-PEG₂₂₀₀]₃₂

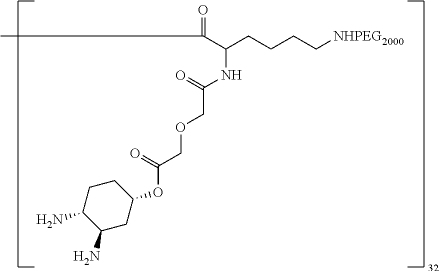

Prepared as per the procedure for synthesis of BHALys[Lys]32[α-diglycolic acid-5R-oxy-(1R,2R)-diaminocyclohexane]₃₂[ε-PEG2200]₃₂. HPLC: Rf (min)=7.42. ¹H NMR (CD₃OD, 300 MHz) δ (ppm): 1.30-2.84 (m, 659H), 3.21-3.27 (m, 87H), 3.37 (s, 96H), 3.40-4.01 (m, 5951H), 4.13-4.47 (m, 194H), 5.23 (s, 30H), 7.26-7.35 (m, 10H). ¹H NMR suggests 32 linkers/dendrimer. Actual molecular weight is approximately 86.3 kDa.

### (c) Preparation of BHALys[Lys]₃₂[α-diglycolic acid-5S-oxy-(1R,2R)-diaminocyclohexane-dichloroPlatinum (II)]₃₂[ε-PEG₂₂₀₀]₃₂

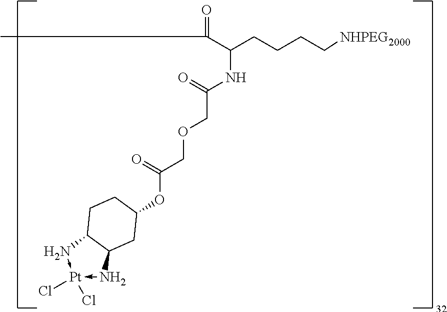

Prepared as per the procedure for synthesis of BHALys[Lys]₃₂[α-diglycolic acid-5R-oxy-(1R,2R)-diaminocyclohexane-dichloroPlatinum (II)]₃₂[ε-PEG₂₂₀₀]₃₂. HPLC: Rf (min)=7.59. ¹H NMR (CD₃OD, 300 MHz) δ (ppm): 1.27-2.40 (m, 518H), 3.20-3.28 (m, 67H), 3.37 (s, 102H), 3.40-4.02 (m, 5949H), 4.14-4.67 (m, 171H), 7.12-7.48 (m, 13H). Actual molecular weight is approximately 94.8 kDa.

### (d) Preparation of BHALys[Lys]₃₂[α-diglycolic acid-5S-oxy-(1R,2R)-diaminocyclohexane-oxalato-Platinum (II)]₃₂[ε-PEG₂₂₀₀]₃₂

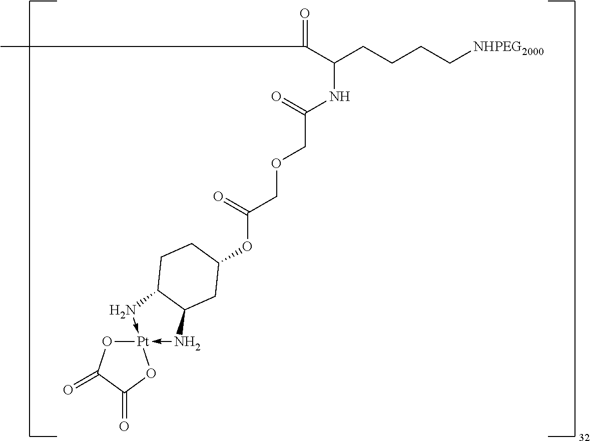

Prepared as per the procedure for synthesis of BHALys[Lys]₃₂[α-diglycolic acid-5R-oxy-(1R,2R)-diaminocyclohexane-oxalato-Platinum (II)]₃₂[C-PEG₂₂₀₀]₃₂. HPLC: Rf (min)=7.52. ¹H NMR (CD₃OD, 300 MHz) δ (ppm): 1.25-2.39 (m, 472H), 3.37 (s, 122H), 3.40-4.02 (m, 5918H), 4.22-4.69 (m, 142H), 7.15-7.65 (m, 12H). ICP-OES: Determined platinum content was 5.8%. This indicates there are 28 platinum atoms (in platinum containing moiety) on about 44% of the surface amino groups. Actual molecular weight is approximately 94.2 kDa.

### Example 15

### Preparation of BHALys[Lys]₃₂[α-diglycolato[(1R,2R)-cyclohexane-1,2-diamine-N,N′]-acetatooxalatoplatinum(IV)]₃₂[ε-PEG₂₂₀₀]₃₂ (Compound 15)

### (a) Preparation of Acetato[(1R,2R)-cyclohexane-1,2-diamine-N,N′]-diglycolatooxalatoplatinum(IV)

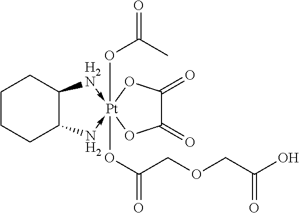

To a solution of acetate[(1R,2R)-cyclohexane-1,2-diamine-N,N′]-hydroxidooxalatoplatinum(IV) (200 mg, 0.42 mmol) (prepared according to Chemistry-A European Journal 19(5), 1672-1676, 2013) in dimethylformamide (10 mL) was added diglycolic anhydride (49 mg, 0.42 mmol). The reaction mixture was heated at 75° C. in the dark for 15 h. Any remaining solids were removed by filtration and the volatiles were removed over rotary evaporator. to give the crude product which was washed with ethyl acetate to afford a light brown solid (220 mg, 88%). LCMS (C18 column): R_{f} (min)=7.79. ESI (+ve) observed [M+H]⁺=590. Calculated for C₁₄H₂₂N₂O₁₁Pt=589.41 Da. ¹H NMR (D₂O, 300 MHz) δ (ppm): 1.14-1.19 (m, 2H), 1.41-1.59 (m, 4H), 1.98-1.99 (m, 3H), 2.18-2.23 (m, 2H), 2.75-2.86 (m, 2H), 4.14-4.19 (m, 4H).

### (b) Preparation of BHALys[Lys]₃₂[α-diglycolato[(1R,2R)-cyclohexane-1,2-diamine-N,N′]-acetatooxalatoplatinum(IV)]₃₂[ε-PEG₂₂₀₀]₃₂

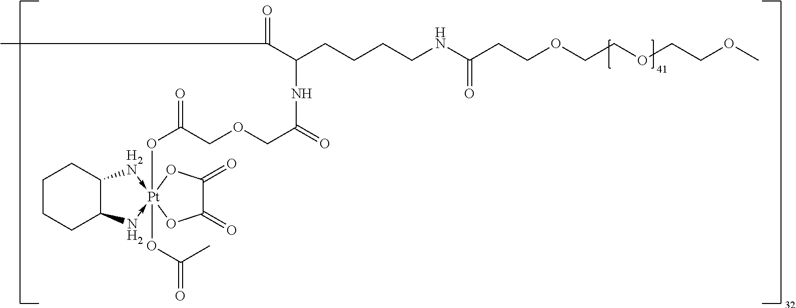

To a solution of acetato[(1R,2R)-cyclohexane-1,2-diamine-N,N′]-diglycolatooxalatoplatinum(IV) (68 mg, 0.12 mmol) in dimethylformamide (3 mL) was added N-methylmorpholine (25 μL, 0.23 mmol) and benzotriazol-1-yl-oxytripyrrolidinophosphonium hexafluorophosphate (PyBOP) (60 mg, 0.12 mmol). The mixture was stirred at room temperature for 10 min. before a solution of BHALys[Lys]₁₆[Lys(NH₂.TFA)₃₂(PEG2000)₃₂] (199 mg, 0.0024 mmol) and N-methylmorpholine (25 μL, 0.23 mmol) in dimethylformamide (3 mL) was added dropwise. The reaction mixture was stirred for 15 hours at room temperature under nitrogen atmosphere. Volatiles were evaporated over rotary evaporator and the residue taken up in water and purified via ultrafiltration (10 MWCO). The retentate was collected and freeze dried to provide 189 mg (84%) of the platinum coordinated product. HPLC (C8, gradient: 5-80% ACN/H₂O (1-7 min), 80% ACN (7-9 min), 80-5% ACN (9-11 min), 5% ACN 11-15 min), 0.1% HCOONH₄R_{f} (min)=8.50. ¹H NMR (CD₃OD, 300 MHz) δ (ppm): 1.10-2.05 (m, 446H), 1.97 (brs, 92H), 2.19-2.58 (m, 83H), 2.63-3.06 (m, 111H), 3.37 (s, 120H), 3.40-4.02 (m, 5859H), 4.11-4.63 (m, 159H) 7.24-7.44 (m, 14H). ICP-OES: Determined Pt % 5.0%. This indicates there are 24 platinum atoms (in platinum containing moiety) on about 38% of the surface amino groups. Actual molecular weight of conjugate is determined to be 92.5 KDa.

### Example 16

### Preparation of BHALys[Lys]₃₂[α-diglycolato[(1R,2R)-cyclohexane-1,2-diamine-N,N′]-hydroxidooxalatoplatinum(IV)]₃₂[ε-PEG₂₂₀₀]₃₂ (Compound 16)

### (a) Preparation of Diglycolato[(1R,2R)-cyclohexane-1,2-diamine-N,N′]-hydroxidooxalatoplatinum(IV)

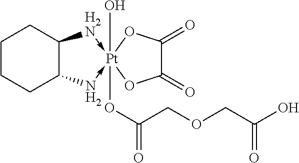

To a suspension of [(1R,2R)-cyclohexane-1,2-diamine-N,N′]-dihydroxidooxalato-platinum(IV)² (25 mg, 0.06 mmol) in dimethylsulfoxide (1.5 mL) was added diglycolic anhydride (6.7 mg, 0.06 mmol). The reaction mixture was stirred in the dark for 15 h. Any remaining solids were removed by filtration and dimethylsulfoxide was removed by lyophilisation to give the crude product which was purified by preparative high performance liquid chromatography providing diglycolato[(1R,2R)-cyclohexane-1,2-diamine-N,N′]-hydroxidooxalatoplatinum(IV) (2.3 mg, 7%). Conditions: XBridge C18 column with multiple gradient H₂O (1-5 min), 0-10%% ACN/H₂O (5-30 min), 10% ACN/H₂O (30-37 min), 10-60% ACN/H₂O (37-38 min), 60% ACN/H₂O (38-43 min), 60-0% ACN/H₂O (43-44 min), H₂O (44-55 min) with 0.1% HCOOH. LCMS (C18 column): R_{f} (min)=6.02. ESI (+ve) observed [M]⁺=547. Calculated for C₁₂H₂₀N₂O₁₀Pt=547.37 Da. ¹H NMR (D₂O, 300 MHz δ (ppm)): 1.13-1.23 (m, 2H), 1.41-1.57 (m, 4H), 2.18-2.22 (m, 2H), 2.77-2.87 (m, 2H), 4.02-4.15 (m, 4H).

### (b) Preparation of BHALys[Lys]₃₂[α-diglycolato[(1R,2R)-cyclohexane-1,2-diamine-N,N′]-hydroxidooxalatoplatinum(IV)]₃₂[ε-PEG₂₂₀₀]₃₂

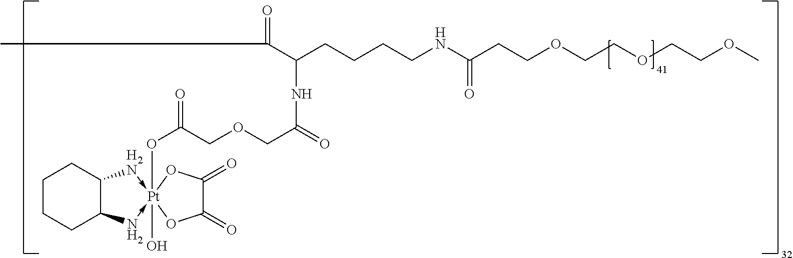

Prepared as per the procedure for synthesis of BHALys[Lys]₃₂[α-diglycolato[(1R,2R)-cyclohexane-1,2-diamine-N,N′]-acetatooxalatoplatinum(IV)]₃₂[ε-PEG₂₂₀₀]₃₂. HPLC: Rf (min)=7.72. ¹H-nmr (300 MHz, CD₃OD) δ (ppm): 1.12-2.06 (m, 298H), 2.12-3.04 (m, 55H), 3.37 (s, 132H), 3.40-4.01 (m, 5900H), 4.16-4.69 (m, 246H) 7.17-7.46 (m, 9H). ICP-OES: Determined Pt % 4.4%. This indicates there are 20 Pt (in platinum containing moiety) on about 31% of the surface amino groups. Actual molecular weight of conjugate is determined to be 89.6 KDa.

### Example 17

### Cell Inhibition Studies

Cell growth inhibition was determined using the Sulforhodamine B (SRB) assay (Voigt W., “Sulforhodamine B assay and chemosensitivity” Methods Mol. Med., 2005, 110, 39-48) against various cancer cell lines after 72 hours. GI₅₀ is the concentration required to inhibit total cell growth by 50% as per NCI standard protocols.

All solutions were prepared in saline and were stored at −20° C. All values were based on equivalent platinum loading except standard drugs which were calculated on total molecular weight. The results are shown in Table 1 in the micromolar range.

TABLE 1
Efficacy in an in vitro cell line model
GI₅₀ (μM)

| | Cell line | | | | |
|---|---|---|---|---|---|
| | OE21 | | | FADU | |
| | (head and | 2008 | LoVo | (head and | SW620 |
| Compound | neck) | (ovarian) | (colon) | neck) | (colon) |
| Carboplatin | 3.6 | 4.9 | 23 | 20 | 33.5 |
| Cisplatin | 0.4 | 0.5 | 2.8 | 2.0 | 3.6 |
| Oxaliplatin | 1.0 | 0.2 | 0.5 | 0.9 | 0.2 |
| 1 | | 4.5 | 7.5 | 9.0 | 3.5 |
| 2 | >30 | 41 | 170 | 200 | >300 |
| 3 | | 69 | >300 | >300 | >300 |
| 4 | 7.4 | 6.4 | 13 | 9.5 | 6.7 |
| 5 | | 2.5 | 3.0 | 3.0 | 1.5 |
| 6 | 14.5 | 3.9 | 8.1 | 12 | 4.2 |
| 7 | >30 | 36 | 220 | 150 | 170 |
| 8 | 26.5 | 18 | 72 | 71 | 63 |
| 12 | | 6.0 | 5.0 | 8.5 | 2.5 |

### Example 18

### Maximum Tolerated Dosage

Groups of female Balb/c mice were administered an intravenous injection of macromolecule or oxaliplatin (0.1 mL/10 g body weight) solubilised in 5% glucose once weekly for three weeks (day 1, 8 and 15). Mice were weighed daily and watched for signs of toxicity. Animals were monitored for up to 10 days following the final drug dose. Any mice exceeding ethical endpoints (>20% body weight loss, poor general health) were immediately sacrificed and observations were noted. The results are shown in Table 2 and demonstrate that the mice can tolerate increased doses of platinum equivalents in the macromolecules. More than 1.5 times the dose of platinum could be safely administered using the macromolecules compared to oxaliplatin alone.

TABLE 2
Maximum tolerated dose determined in an in vitro animal model

| | Doses Tested | Tolerated Dose |
|---|---|---|
| Drug | (mg/Kg Pt equivalents) | (mg/Kg Pt equivalents) |
| Oxaliplatin | 2.5, 3.5, 5 | 5 |
| Compound 1 | 50, 20, 15, 10, 7.5 | 7.5 |
| Compound 5 | 10, 7.5 | 10 |
| Compound 12 | 60, 50, 35, 25, 15, 12.5, 10, 8 | >60 |
| Compound 13 | 35, 25, 20, 15, 10 | >35 |
| Compound 14 | 35, 25, 20, 15, 10 | >35 |

### Example 19

### Xenograft Model Efficacy Study

Female Balb/c nude mice (aged 7 weeks) were inoculated subcutaneously on the flank with 4×10⁶ SW620 cells in PBS-matrigel (1:1). Thirteen days later, 50 mice with similar sized tumors (˜110 mm³) were randomized into 4 groups (n=12). All compounds were dissolved in 5% glucose at the concentration required to achieve administration of 0.1 mL/10 g body eight. Each treatment group was administered (Vehicle, Oxaliplatin 5 mg/kg, Compound 1 8.5 mg/kg, Compound 5, 9 mg/kg (All Pt equivalents) by intravenous injection. All treatments were administered once weekly for three weeks (days 1, 8 and 15) at 0.1 mL/10 g body weight. Mice were sacrificed if ethical endpoint (weight loss of greater than 20% or tumour size greater than 1200 mm³) was met. Results shown in Table 3 show that the macromolecules were more effective in suppressing tumor growth for longer than oxaliplatin.

TABLE 3
Efficacy determined in an in vivo animal xenograft model

| | Mean Tumor Volume mm³ (SEM) | | | | | | | |
|---|---|---|---|---|---|---|---|---|
| Day | Vehicle | SEM | Oxaliplatin | SEM | Comp 1 | SEM | Comp 5 | SEM |
| 1 | 100.50 | 7.69 | 100.56 | 7.40 | 100.47 | 7.88 | 100.96 | 8.04 |
| 7 | 201.84 | 11.45 | 203.06 | 13.33 | 184.55 | 7.95 | 196.01 | 14.87 |
| 14 | 343.41 | 26.80 | 340.50 | 25.06 | 283.02 | 16.46 | 303.00 | 25.60 |
| 21 | 551.33 | 34.12 | 501.57 | 37.38 | 316.90 | 18.40 | 363.01 | 31.65 |
| 28 | 787.35 | 75.94 | 755.89* | 60.41* | 345.48* | 67.08* | 531.12 | 57.00 |

*n = <12 as ethical endpoint reached for 1 or more animal.

### Example 20

### Toxicity Study

Female Balb/c nude mice (age 7 weeks) had subcutaneous tumors established as described in Example 15. The mice were randomised into 4 groups of n=5 mice, (mean tumor volume ˜90 mm³) On day one, animals were eye bled in the morning for baseline cell counts and then drug dosing commenced later that day. Drug dosing was performed on days 1, 8 and 15 at the previously determined maximum tolerated doses. All compounds were dissolved in 5% glucose at the concentration required to allow administration of 0.1 mL/10 g body weight. Each treatment group was administered (Vehicle, Oxaliplatin 5 mg/kg, Compound 1 8.5 mg/kg, Compound 5, 9 mg/kg (All Pt equivalents)) by intravenous injection. Subsequent eye bleeds were performed on days 7, 14 and 26 (Tables 4 A-D, in each case data represented as mean ratio of counts for each time point relative to that at the baseline).

TABLE 4A
Effect on mean white blood cell count in an in vivo animal xenograft
model Relative mean white blood cell count (baseline corrected)

| | | Oxaliplatin | Compound 1 | Compound 5 |
|---|---|---|---|---|
| Day | Vehicle (SEM) | (SEM) | (SEM) | (SEM) |
| 1 | 1.000 (0.147) | 1.000 (0.087) | 1.000 (0.094) | 1.000 (0.238) |
| 7 | 0.646 (0.105) | 0.214 (0.057) | 1.238 (0.195) | 0.566 (0.151) |
| 14 | 1.278 (0.198) | 0.230 (0.062) | 1.754 (0.249) | 1.153 (0.236) |
| 26 | 0.596 (0.167) | 0.354 (0.085) | 0.944 (0.100) | 1.093 (0.245) |

TABLE 4B
Effect on neutrophil count in an in vivo animal xenograft model
Relative mean neutrophils (baseline corrected)

| | | Oxaliplatin | Compound 1 | Compound 5 |
|---|---|---|---|---|
| Day | Vehicle (SEM) | (SEM) | (SEM) | (SEM) |
| 1 | 1.000 (0.199) | 1.000 (0.153) | 1.000 (0.126) | 1.000 (0.333) |
| 7 | 0.776 (0.137) | 0.135 (0.044) | 1.085 (0.162) | 1.216 (0.316) |
| 14 | 0.928 (0.143) | 0.130 (0.070) | 1.470 (0.237) | 1.293 (0.308) |
| 26 | 0.620 (0.222) | 0.277 (0.077) | 0.743 (0.128) | 0.710 (0.376) |

TABLE 4C
Effect on lymphocyte count in an in vivo animal xenograft model
Mean lymphocytes counts (baseline corrected)

| | | Oxaliplatin | Compound 1 | Compound 5 |
|---|---|---|---|---|
| Day | Vehicle (SEM) | (SEM) | (SEM) | (SEM) |
| 1 | 1.000 (0.127) | 1.000 (0.125) | 1.000 (0.196) | 1.000 (0.381) |
| 7 | 0.518 (0.118) | 0.240 (0.130) | 1.365 (0.420) | 0.300 (0.150) |
| 14 | 1.145 (0.284) | 0.302 (0.066) | 2.059 (0.500) | 1.057 (0.340) |
| 26 | 0.551 (0.204) | 0.380 (0.154) | 1.127 (0.221) | 1.224 (0.366) |

TABLE 4D
Effect on platelet count in an in vivo animal xenograft model
Relative Mean platelet count (baseline corrected)

| | | Oxaliplatin | Compound 1 | Compound 5 |
|---|---|---|---|---|
| Day | Vehicle (SEM) | (SEM) | (SEM) | (SEM) |
| 1 | 1.000 (0.123) | 1.000 (0.082) | 1.000 (0.063) | 1.000 (0.048) |
| 7 | 0.978 (0.099) | 0.421 (0.055) | 1.031 (0.117) | 1.358 (0.131) |
| 14 | 1.245 (0.112) | 0.469 (0.189) | 1.279 (0.131) | 1.186 (0.144) |
| 26 | 1.122 (0.118) | 0.380 (0.080) | 0.804 (0.103) | 0.854 (0.128) |

### Example 21

### Nucleated Cells in Bone Marrow

SW-620 tumour bearing male nude mice were dosed with vehicle, Compound 1 (10 mg/kg Pt equivalents, IV) or Oxaliplatin (5 mg/kg Pt equivalents, IV) by intravenous infusion. Mouse femur was flushed of bone marrow with PBS, the cells aspirated then stained with a suitable nuclear stain, such as acridine orange, and assessed via flow cytometry for nucleated cells at 6 and 72 hours (n=5) after dosing.

TABLE 5
Effect on bone marrow nucleated cells in an in vivo animal
xenograft model

| | % nucleated cells relative to control | | |
|---|---|---|---|
| | Control | Oxaliplatin | Compound 1 |
| 6 hours | 1.00 | 0.91 | 0.86 |
| 72 hours | 1.00 | 0.54 | 0.73 |

Table 5 shows that use of Compound 1 resulted in a higher proportion of nucleated cells at 72 hours demonstrating that it is less damaging to the nucleated cells of the bone marrow (being leukocytes such a neutrophils, erythroblasts, lymphocytes, eosinophils and monocytes) than Oxaliplatin at greater doses of Pt.

### Example 22

### Neuropathic Responses

Balb/c female mice received Oxaliplatin (1.75 mg/kg Pt Equivalents IV) or Compound 1 (1.75 mg/kg Pt Equivalents IV) weekly (n=8) at days 0 and 7 by intravenous infusion. All compounds were dissolved in 5% glucose at the concentration required to achieve the intended dose. Two days after the second dose (day 9), and then one week later (day 16), mice were tested for mechanical sensitivity using the Von Frey test. A low score indicates increased mechanical sensitivity as measured by the gram force of the smallest fibre to elicit three hind paw withdrawal responses to five applications of the filament.

TABLE 6
Effect on neuropathic response in an in vivo animal model

| | Oxaliplatin | Compound 1 |
|---|---|---|
| Baseline | 1.3 | 1.25 |
| Day 9 | 0.68 | 0.8 |
| Day 21 | 0.49 | 0.75 |

Results in table 6 show mice receiving compound 1 had improved mechanical sensitivity after 2 doses demonstrating less peripheral neurotoxicity.

1. A macromolecule comprising:
i) a dendrimer comprising a core and at least one generation of building units, wherein the outermost generation of building units comprises surface amino groups; and
ii) one or more platinum-containing moiety;
wherein one or more of the surface amino groups are each independently attached to a platinum coordinating group (P_{C}) of platinum complex (-Pc-P_{M}), and wherein the platinum coordinating group (P_{C}) and the platinum-containing moiety (P_{M}) together form a six coordinate platinum (IV) complex.

2. The macromolecule of claim 1, wherein the platinum coordinating group (P_{C}) provides a monodentate ligand in the platinum complex (-Pc-P_{M}).

3. (canceled)

4. (canceled)

5. The macromolecule of claim 1, wherein one or more of the surface amino groups are each independently attached to a pharmacokinetic modifying agent.

6. The macromolecule of claim 5, wherein the pharmacokinetic modifying agent is polyethylene glycol with a molecular weight in the range of 1000 to 2500 Da.

7. The macromolecule of claim 1, further comprising one or more targeting groups attached to one or more surface amino groups of the dendrimer or to a functional group on the core of the dendrimer.

8. The macromolecule of claim 7, wherein the targeting agent is an antibody or antibody fragment or mimetic.

9. The macromolecule of claim 1, wherein the dendrimer has 2 to 6 generations of building units of lysine or lysine analogues and an average molecular weight in the range of 60 to 110 kDa.

10. The macromolecule of claim 1, wherein there are platinum-containing moieties attached to at least about 33% of the surface amino groups.

11. The macromolecule of claim 1, selected from a macromolecule of Formula 1:
D_{C}-[D_{B}]_{b}-[D_{S}]ₛ-[L]_{d}-[T]_{z} Formula 1
wherein:
D_{C} represents a core moiety comprising at least two functional groups linked to at least two subsurface building units D_{B};
D_{B} represents two or more subsurface building units each comprising at least three branching point functional groups, wherein one branching point functional group is attached to one of the functional groups of the core or a previous generation subsurface building unit, and at least two functional groups are each independently attached to a next generation of building units; and b is an integer between 2 and 62;
D_{S} represents four or more surface building units wherein each building unit comprises one or more surface amino groups; and s is an integer between 4 and 64;
L is an optional linker group that is attached to the surface amino group of the dendrimer; wherein d is 0 to 128;
T represents one or more terminal groups each independently attached to a surface amino group of the surface building units (Ds), optionally by a linker group L, wherein the one or more terminal groups are selected from at least a first terminal group and optionally a second terminal group, wherein:
the first terminal group is the platinum complex -Pc-P_{M} wherein a platinum coordinating group (P_{C}) is attached to the surface amino group, optionally by the linker group L, and further coordinated to a platinum atom of the platinum containing moiety (P_{M});
the second terminal group is selected from at least one of a pharmacokinetic modifying agent (M), a targeting agent, and a blocking agent; and
z is an integer between 1 and 128.

12. The macromolecule according to claim 11, wherein the ratio of platinum-containing moieties P_{M} and pharmacokinetic modifying agents M is in the range of 1:4 to 4:1.

13. The macromolecule of claim 11, wherein the first terminal group is a platinum complex [-Pc-P_{M}]ₓ comprising the platinum coordinating group (P_{C}) attached to the surface amino group, wherein the platinum coordinating group (P_{C}) is further coordinated as a monodentate ligand to a platinum atom of the platinum containing moiety (P_{M}); and a second terminal group is a pharmacokinetic modifying agent [M]_{y}; and x and z are integers between 1 and 128, and y is an integer between 0 and 127.

14. The macromolecule of claim 13, wherein b, s, x and y are together selected from (14, 16, 16, 16), (30, 32, 32, 32) or (62, 64, 64, 64).

15. The macromolecule of claim 11, wherein the second terminal group is a pharmacokinetic modifying agent (M) and a targeting agent.

16. The macromolecule of claim 2, wherein the monodentate ligand has a functional group selected from the group consisting of carboxylate, amide, amine, and hydroxo for coordination to the platinum atom of the platinum containing moiety (P_{M}).

17. The macromolecule of claim 16, wherein the platinum coordinating group is selected from: 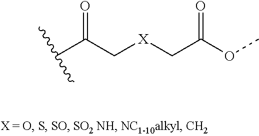
wherein the dashed line indicates coordination of the monodentate ligand to the platinum atom of the platinum containing moiety and the wavy line indicates attachment to a surface amino group of the dendrimer or an optional linking group to the surface amino group of the dendrimer.

18. The macromolecule of claim 1, wherein the platinum containing moiety comprises one or more monodentate ligands independently selected from the group consisting of amine, amide, cyano carboxylate, hydroxyl, and halo groups, and optionally one or two bidentate ligands independently selected from the group consisting of diamine, dicarboxylate and amine carboxylate groups.

19. The macromolecule of claim 18, wherein the platinum-containing moiety is selected from the group consisting of: 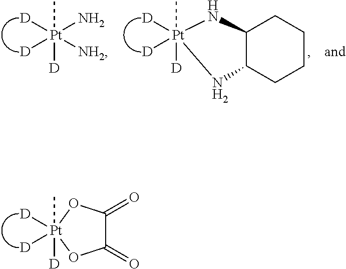
wherein the dashed line indicates coordination to the platinum coordinating group, -D-D- is a bidentate ligand selected from the group consisting of diamine, dicarboxylate and amine carboxylate, and D- is a monodentate ligand selected from the group consisting of amine, carboxylate, hydroxyl and halo.

20. The macromolecule of claim 19, wherein the platinum-containing moiety is selected from: 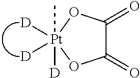
wherein the dashed line indicates coordination to the platinum coordinating group, -D-D- is a bidentate ligand selected from a diamine group, and D- is a monodentate ligand selected from the group consisting of amine, carboxylate, hydroxyl and halo.

21. (canceled)

22. The macromolecule of claim 1, wherein the six coordinate platinum(IV) complex comprises oxaliplatin or a derivative or moiety thereof.

23. The macromolecule of claim 1, which comprises a six coordinate platinum(IV) complex of: 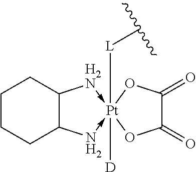
wherein
L is a linker group and the wavy line indicates attachment to a surface amino group of the dendrimer, and
D is a monodentate ligand.

24. The macromolecule of claim 23, wherein D is selected from the group consisting of halo, amine, amide, carboxyl, carboxylate, cyano, and hydroxo

25. The macromolecule of claim 1, wherein each platinum coordinating group is attached to a surface amino group of the dendrimer through a linker group L.

26. The macromolecule of claim 25, wherein the linker group L comprises a group of Formula 3:
—C(O)—X—C(O)—W-E Formula 3
wherein one —C(O)— group forms an amide bond with a surface amino group of the dendrimer and the other —C(O)— group, W or E, is attached to the platinum coordinating group PC, optionally through a peptide, W; and
X is selected from —C₁-C₁₀alkylene-, —(CH₂)_{q}-A-(CH₂)ₜ— and Q;
A is selected from —O—, —S—, —NR₁—, —N⁺(R₁)₂—, —S—S—, —[OCH₂CH₂]ᵣ—O—, —Y—, and —O—Y—O—;
Q is selected from Y or —Z═N—NH—S(O)_{w}—Y—;
Y is selected from cycloalkyl, heterocycloalkyl, aryl and heteroaryl;
Z is selected from —(CH₂)ₐ—C(CH₃)═, —(CH₂)ₐCH═, cycloalkyl and heterocycloalkyl;
R₁ is selected from hydrogen and C₁-C₄ alkyl;
r, q and t are independently selected from 1, 2, 3 and 4;
w is selected from 0, 1 and 2;
a is selected from 1, 2, 3 and 4;
W is absent or is an amino acid residue or a peptide of 1 to 10 amino acid residues; and
E is absent or is selected from —O—, —S—, —NR₁—, —N⁺(R₁)₂—, —S—S—, —[OCH₂CH₂]ᵣ—O—, —Y—, and —O—Y—O—.

27. A macromolecule according to claim 26, wherein X is selected from —C₁₋₆alkylene-, —CH₂-A-CH₂— and —CH₂CH₂-A-CH₂CH₂—.

28. A macromolecule according to claim 26, wherein X is selected from —CH₂CH₂—, —CH₂CH₂CH₂—, —CH₂OCH₂— and —CH₂SCH₂—.

29. A macromolecule according to claim 26 wherein the peptide is selected from -GGG-, -GFLG-, GLFG-, -GILGVP- and PVGLIG-.

30. A pharmaceutical composition comprising the macromolecule of claim 1 and a pharmaceutically acceptable carrier.

31. A method of treating or suppressing the growth of a cancer comprising administering an effective amount of the macromolecule of claim 1 or pharmaceutical composition comprising the macromolecule and a pharmaceutically acceptable carrier.

32. A method according to claim 31 wherein the cancer is a tumor of the prostate, testes, lung, kidney, bladder, colon, pancreas, bone, spleen, liver, brain, head and/or neck, breast, gastrointestinal tract, skin, cervix or ovary.

33. A method of reducing the side effects of a platinum-containing oncology drug comprising administering the macromolecule of claim 1 or a pharmaceutical composition comprising the macromolecule and a pharmaceutically acceptable carrier.

34. A method according to claim 33 wherein the side effect that is reduced is hematologic toxicity, neurological toxicity, gastrointestinal toxicity, pulmonary toxicity, cardiotoxicity, hepatotoxicity, nephrotoxicity, ototoxicity or encephalotoxicity.
